(19) United States
(12) Patent Application Publication
(10) Pub. No.: US 2010/0130477 A1
(43) Pub. Date: May 27, 2010

(54) Spirocyclobutyl Piperidine Derivatives

(75) Inventors: Peter Bernstein, Wilmington, DE (US); Dean Brown, Wilmington, DE (US); Andrew Griffin, Montreal (CA); Maxime C. Tremblay, Montreal (CA); Steven Wesolowski, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/623,585

(22) Filed: Nov. 23, 2009

Related U.S. Application Data
(60) Provisional application No. 61/117,768, filed on Nov. 25, 2008.

(51) Int. Cl. A61K 31/551; C07D 487/00; C07D 223/14; A61K 31/497; A61P 25/28; A61P 25/18; A61P 25/22; A61P 25/00; A61P 3/04

(52) U.S. Cl. 514/218; 544/230; 540/543; 514/253.09

(57) ABSTRACT

Disclosed herein is at least one spirocyclobutyl piperidine derivative, at least one pharmaceutical composition comprising at least one spirocyclobutyl piperidine derivative disclosed herein, and at least one method of using at least one spirocyclobutyl piperidine derivative disclosed herein for treating at least one histamine H3 receptor associated condition therewith.

Disclosed herein is at least one spirocyclobutyl piperidine derivative, at least one pharmaceutical composition comprising at least one spirocyclobutyl piperidine derivative disclosed herein, and at least one method of using at least one spirocyclobutyl piperidine derivative disclosed herein for treating at least one histamine H3 receptor associated condition therewith.

The histamine H3 receptor is of current interest in developing new medicaments. The H3 receptor is a presynaptic autoreceptor located both in the central and peripheral nervous systems, the skin, and in organs, such as, for example, the lung, the intestine, probably the spleen, and the gastrointestinal tract. Recent evidence suggests the H3 receptor has intrinsic, constitutive activity in vitro as well as in vivo (i.e., it is active in the absence of an agonist). Compounds acting as inverse agonists can inhibit this activity. The histamine H3 receptor has been shown to regulate the release of histamine and also of other neurotransmitters, such as, for example, serotonin and acetylcholine. Some histamine H3 ligands, such as, for example, a histamine H3 receptor antagonist or inverse agonist may increase the release of neurotransmitters in the brain, whereas other histamine H3 ligands, such as, for example, histamine H3 receptor agonists may inhibit the biosynthesis of histamine, as well as, inhibit the release of neurotransmitters. This suggests that histamine H3 receptor agonists, inverse agonists, and antagonists could mediate neuronal activity. As a result, efforts have been undertaken to develop new therapeutics that target the histamine H3 receptor.

Described herein are compounds of formula I, or pharmaceutically acceptable salts of formula I, or mixtures thereof:

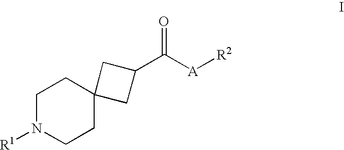

wherein

A is

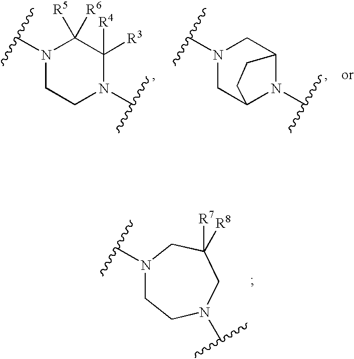

R¹ is aryl, 5- or 6-membered heteroaryl, —S(═O)₂R⁹, —C(═O)R¹⁰, or —C(═O)NR¹¹R¹²;

R² is C₃₋₆cycloalkyl or C₁₋₆alkyl;

R³, R⁴, R⁵, R⁶, R⁷, and R⁸ are each, independently, selected from hydrogen and —C₁-C₃alkyl;

R⁹, R¹⁰ and R¹¹ are each, independently, selected from C₁₋₆alkyl, 4-membered heterocycloalkyl, 5-membered heterocycloalkyl, 6-membered heterocycloalkyl, 7-membered heterocycloalkyl, C₃₋₇cycloalkyl, aryl, 5-membered heteroaryl, 6-membered heteroaryl, (C₁₋₃ alkyl)-(5-membered heteroaryl), and (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;

R¹² is H or C₁₋₆alkyl; and

R¹³ and R¹⁴ are each, independently, selected from H and C₁₋₃alkyl.

Further described herein is at least one compound according to formula I, or pharmaceutically acceptable salts of formula I, or mixtures thereof for use as a medicament.

Even further described herein is the use of at least one compound of formula I, or pharmaceutically acceptable salts of formula I, or mixtures thereof in the manufacture of a medicament for the therapy of at least one disorder selected from cognitive deficit in schizophrenia, narcolepsy, obesity, Attention deficit hyperactivity disorder, and Alzheimer's disease.

Yet even further described herein is a pharmaceutical composition comprising at least one compound according to formula I, or pharmaceutically acceptable salts of formula I, or or mixtures thereof and a pharmaceutically acceptable carrier and/or diluent.

Still even further described herein is a method for treating at least one disorder selected from cognitive deficit in schizophrenia, narcolepsy, obesity, attention deficit hyperactivity disorder, and Alzheimer's disease in a warm-blooded animal, comprising administering to said animal in need of such treatment a therapeutically effective amount of at least one compound according to formula I, or pharmaceutically acceptable salts of formula I, or mixtures thereof.

Still yet even further described herein is a method for treating a disorder in which modulating the histamine H3 receptor is beneficial comprising administering to a warm-blooded animal in need of such treatment a therapeutically effective amount of at least one compound according to formula I, or pharmaceutically acceptable salts of formula I, or mixtures thereof.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Unless specifically stated otherwise herein, references made in the singular may also include the plural. For example, “a” and “an” may refer to either one, or one or more.

Embodiments identified herein as exemplary are intended to be illustrative and not limiting.

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Definitions of terms used in describing the invention are set forth hereinbelow. Unless otherwise indicated, the initial definition provided for a group or term applies each time such group or term is used individually or as part of another group.

Throughout the specification, groups and substituents thereof may be chosen by one skilled in the field to provide stable moieties and compounds.

Unless specified otherwise herein, the nomenclature used herein generally follows the examples and rules stated in Nomenclature of Organic Chemistry, Sections A, B, C, D, E, F, and H, Pergamon Press, Oxford, 1979.

The term “Cₘ-Cₙ” or “Cₘ-Cₙ group” used alone or as a prefix, refers to any group having m to n carbon atoms. For example, the term “C₁-C₄alkyl” refers to an alkyl group containing 1, 2, 3, or 4 carbon atoms.

The terms “alkyl” and “alk” refer to a straight or branched chain alkane (hydrocarbon) radical containing from 1 to 12 carbon atoms. Exemplary “alkyl” and “alk” groups include, but are not limited to, for example, methyl; ethyl; propyl; isopropyl; 1-methylpropyl; n-butyl, t-butyl; isobutyl; pentyl; hexyl; isohexyl; heptyl; 4,4-dimethylpentyl; diethylpentyl; octyl; 2,2,4-trimethylpentyl; nonyl; decyl; undecyl; and dodecyl.

The term “hydrocarbon” refers to a chemical structure comprising only carbon and hydrogen atoms.

The term “hydrocarbon radical” refers to a hydrocarbon that has had at least one hydrogen removed therefrom.

The term “lower alkyl” refers to an alkyl group containing from 1 to 4 carbon atoms. It is of import to note that the term “lower alkyl” is encompassed within the definition of “alkyl”. The usage of the term “lower alkyl”, however, is not intended to limit the definition of the term “alkyl” either explicitly or implicitly to a straight- or branched-chain saturated hydrocarbon radical containing from 5 to 12 carbon atoms. Exemplary lower alkyl groups include, but are not limited to, for example, methyl; ethyl; propyl; isopropyl; n-butyl; t-butyl; and isobutyl.

The term “aryl” refers to monocyclic or bicyclic aromatic hydrocarbon rings having from 6 to 12 carbon atoms in the ring portion. Exemplary aryl groups include but are not limited to, for example, phenyl; phen-1-yl-2-yl; phen-1-yl-3-yl; phen-1-yl-4-yl; phen-1-yl-5-yl; phen-1-yl-6-yl; naphthalenyl; naphthalen-1-yl-2-yl; naphthalen-1-yl-3-yl; naphthalen-1-yl-4-yl; naphthalen-1-yl-5-yl; naphthalen-1-yl-6-yl; naphthalen-1-yl-7-yl; naphthalen-1-yl-8-yl; naphthalen-2-yl-3-yl; naphthalen-2-yl-4-yl; naphthalen-2-yl-5-yl; naphthalen-2-yl-6-yl; naphthalen-2-yl-7-yl; naphthalen-2-yl-8-yl; naphthalen-3-yl-4-yl; naphthalen-3-yl-5-yl; naphthalen-3-yl-6-yl; naphthalen-3-yl-7-yl; naphthalen-3-yl-8-yl; naphthalen-4-yl-5-yl; naphthalen-4-yl-6-yl; naphthalen-4-yl-7-yl; naphthalen-4-yl-8-yl; naphthalen-5-yl-6-yl; naphthalen-5-yl-7-yl; naphthalen-5-yl-8-yl; naphthalen-6-yl-7-yl; naphthalen-6-yl-8-yl; naphthalen-7-yl-8-yl; biphenyl; biphenyl-2-yl; biphenyl-3-yl; biphenyl-4-yl; biphenyl-5-yl; biphenyl-6-yl; and diphenyl. When two aromatic rings are present, the aromatic rings of the aryl group may either be joined at a single point (e.g., biphenyl), or be fused (e.g., naphthalenyl). Unless reference is made to a specific point of attachment, e.g., as in phen-1-yl-2-yl, naphthalen-1-yl-6-yl, and biphenyl-3-yl, it is intended that such aryl groups can be bonded to at least one other moiety at any available point of attachment.

The term “heteroaryl” refers to aromatic cyclic groups, such as, for example, 5- to 6-membered monocyclic, 7- to 11-membered bicyclic, or 10- to 16-membered tricyclic ring systems having at least one heteroatom in at least one carbon atom-containing ring. Such a heteroaryl may contain 1, 2, 3, or 4 heteroatom(s) selected from nitrogen, oxygen, and sulfur. In one embodiment, such heteroaryl contains 1 or 2 heteroatom(s) selected from nitrogen. The heteroaryl may be attached to another moiety at any available point of attachment, and may be optionally substituted with at least one substituent at any available point of attachment.

Exemplary monocyclic heteroaryl groups include, but are not limited to, for example, pyrazolyl; pyrazol-1-yl; pyrazol-2-yl; pyrazol-3-yl; pyrazol-4-yl; pyrazol-5-yl; pyrazolylyl; pyrazol-1-yl-2-yl; pyrazol-1-yl-3-yl; pyrazol-1-yl-4-yl; pyrazol-1-yl-5-yl; pyrazol-2-yl-3-yl; pyrazol-2-yl-4-yl; pyrazol-2-yl-5-yl; pyrazol-3-yl-4-yl; pyrazol-3-yl-5-yl; pyrazol-4-yl-5-yl; imidazolyl; imidazol-1-yl; imidazol-2-yl; imidazol-3-yl; imidazol-4-yl; imidazol-5-yl; imidazolylyl; imidazol-1-yl-2-yl; imidazol-1-yl-3-yl; imidazol-1-yl-4-yl; imidazol-1-yl-5-yl; imidazol-2-yl-3-yl; imidazol-2-yl-4-yl; imidazol-2-yl-5-yl; imidazol-3-yl-4-yl; imidazol-3-yl-5-yl; imidazol-4-yl-5-yl; triazolyl; triazol-1-yl; triazol-2-yl; triazol-3-yl; triazol-4-yl; triazol-5-yl; triazolylyl; triazol-1-yl-2-yl; triazol-1-yl-3-yl; triazol-1-yl-4-yl; triazol-1-yl-5-yl; triazol-2-yl-3-yl; triazol-2-yl-4-yl; triazol-2-yl-5-yl; triazol-3-yl-4-yl; triazol-3-yl-5-yl; triazol-4-yl-5-yl; oxazolyl; oxazol-2-yl; oxazol-3-yl; oxazol-4-yl; oxazol-5-yl; oxazolylyl; oxazol-2-yl-3-yl; oxazol-2-yl-4-yl; oxazol-2-yl-5-yl; oxazol-3-yl-4-yl; oxazol-3-yl-5-yl; oxazol-4-yl-5-yl; furyl; fur-2-yl; fur-3-yl; fur-4-yl; fur-5-yl; furylyl; fur-2-yl-3-yl; fur-2-yl-4-yl; fur-2-yl-5-yl; fur-3-yl-4-yl; fur-3-yl-5-yl; fur-4-yl-5-yl; thiazolyl; thiazol-1-yl; thiazol-2-yl; thiazol-3-yl; thiazol-4-yl; thiazol-5-yl; thiazolylyl; thiazol-1-yl-2-yl; thiazol-1-yl-3-yl; thiazol-1-yl-4-yl; thiazol-1-yl-5-yl; thiazol-2-yl-3-yl; thiazol-2-yl-4-yl; thiazol-2-yl-5-yl; thiazol-3-yl-4-yl; thiazol-3-yl-5-yl; thiazol-4-yl-5-yl; isoxazolyl; isoxazol-2-yl; isoxazol-3-yl; isoxazol-4-yl; isoxazol-5-yl; isoxazol-2-yl-3-yl; isoxazol-2-yl-4-yl; isoxazol-2-yl-5-yl; isoxazol-3-yl-4-yl; isoxazol-3-yl-5-yl; isoxazol-4-yl-5-yl; pyridyl; pyrid-1-yl; pyrid-2-yl; pyrid-3-yl; pyrid-4-yl; pyrid-5-yl; pyrid-6-yl; pyridylyl; pyrid-1-yl-2-yl; pyrid-1-yl-3-yl; pyrid-1-yl-4-yl; pyrid-1-yl-5-yl; pyrid-1-yl-6-yl; pyrid-2-yl-3-yl; pyrid-2-yl-4-yl; pyrid-2-yl-5-yl; pyrid-2-yl-6-yl; pyrid-3-yl-4-yl; pyrid-3-yl-5-yl; pyrid-3-yl-6-yl; pyrid-4-yl-5-yl; pyrid-4-yl-6-yl; pyrid-5-yl-6-yl; pyridazinyl; pyridazin-1-yl; pyridazin-2-yl; pyridazin-3-yl; pyridazin-4-yl; pyridazin-5-yl; pyridazin-6-yl; pyridazinylyl; pyridazin-1-yl-2-yl; pyridazin-1-yl-3-yl; pyridazin-1-yl-4-yl; pyridazin-1-yl-5-yl; pyridazin-1-yl-6-yl; pyridazin-2-yl-3-yl; pyridazin-2-yl-4-yl; pyridazin-2-yl-5-yl; pyridazin-2-yl-6-yl; pyridazin-3-yl-4-yl; pyridazin-3-yl-5-yl; pyridazin-3-yl-6-yl; pyridazin-4-yl-5-yl; pyridazin-4-yl-6-yl; pyridazin-5-yl-6-yl; pyrimidinyl; pyrimidin-1-yl; pyrimidin-2-yl; pyrimidin-3-yl; pyrimidin-4-yl; pyrimidin-5-yl; pyrimidin-6-yl; pyrimidinylyl; pyrimidin-1-yl-2-yl; pyrimidin-1-yl-3-yl; pyrimidin-1-yl-4-yl; pyrimidin-1-yl-5-yl; pyrimidin-1-yl-6-yl; pyrimidin-2-yl-3-yl; pyrimidin-2-yl-4-yl; pyrimidin-2-yl-5-yl; pyrimidin-2-yl-6-yl; pyrimidin-3-yl-4-yl; pyrimidin-3-yl-5-yl; pyrimidin-3-yl-6-yl; pyrimidin-4-yl-5-yl; pyrimidin-4-yl-6-yl; pyrimidin-5-yl-6-yl; pyrazinyl; pyrazin-1-yl; pyrazin-2-yl; pyrazin-3-yl; pyrazin-4-yl; pyrazin-5-yl; pyrazin-6-yl; pyrazinylyl; pyrazin-1-yl-2-yl; pyrazin-1-yl-3-yl; pyrazin-1-yl-4-yl; pyrazin-1-yl-5-yl; pyrazin-1-yl-6-yl; pyrazin-2-yl-3-yl; pyrazin-2-yl-4-yl; pyrazin-2-yl-5-yl; pyrazin-2-yl-6-yl; pyrazin-3-yl-4-yl; pyrazin-3-yl-5-yl; pyrazin-3-yl-6-yl; pyrazin-4-yl-5-yl; pyrazin-4-yl-6-yl; pyrazin-5-yl-6-yl; triazinyl; triazin-1-yl; triazin-2-yl; triazin-3-yl; triazin-4-yl; triazin-5-yl; triazin-6-yl; triazinylyl; triazin-1-yl-2-yl; triazin-1-yl-3-yl; triazin-1-yl-4-yl; triazin-1-yl-5-yl; triazin-1-yl-6-yl; triazin-2-yl-3-yl; triazin-2-yl-4-yl; triazin-2-yl-5-yl; triazin-2-yl-6-yl; triazin-3-yl-4-yl; triazin-3-yl-5-yl; triazin-3-yl-6-yl; triazin-4-yl-5-yl; triazin-4-yl-6-yl; and triazin-5-yl-6-yl. Unless reference is made to a specific point of attachment, e.g., as in pyrid-2-yl, pyridazin-3-yl, it is intended that such heteroaryl groups can be bonded to at least one other moiety at any available point of attachment.

Exemplary bicyclic heteroaryl groups include, but are not limited to, for example, benzothiazolyl; benzothiazol-1-yl; benzothiazol-2-yl; benzothiazol-3-yl; benzothiazol-4-yl; benzothiazol-5-yl; benzothiazol-6-yl; benzothiazol-7-yl; benzothiazolylyl; benzothiazol-1-yl-2-yl; benzothiazol-1-yl-3-yl; benzothiazol-1-yl-4-yl; benzothiazol-1-yl-5-yl; benzothiazol-1-yl-6-yl; benzothiazol-1-yl-7-yl; benzothiazol-2-yl-3-yl; benzothiazol-2-yl-4-yl; benzothiazol-2-yl-5-yl; benzothiazol-2-yl-6-yl; benzothiazol-2-yl-7-yl; benzothiazol-3-yl-4-yl; benzothiazol-3-yl-5-yl; benzothiazol-3-yl-6-yl; benzothiazol-3-yl-7-yl; benzothiazol-4-yl-5-yl; benzothiazol-4-yl-6-yl; benzothiazol-4-yl-7-yl; benzothiazol-5-yl-6-yl; benzothiazol-5-yl-7-yl; benzothiazol-6-yl-7-yl; benzoxazolyl; benzoxazol-2-yl; benzoxazol-3-yl; benzoxazol-4-yl; benzoxazol-5-yl; benzoxazol-6-yl; benzoxazol-7-yl; benzoxazolylyl; benzoxazol-2-yl-3-yl; benzoxazol-2-yl-4-yl; benzoxazol-2-yl-5-yl; benzoxazol-2-yl-6-yl; benzoxazol-2-yl-7-yl; benzoxazol-3-yl-4-yl; benzoxazol-3-yl-5-yl; benzoxazol-3-yl-6-yl; benzoxazol-3-yl-7-yl; benzoxazol-4-yl-5-yl; benzoxazol-4-yl-6-yl; benzoxazol-4-yl-7-yl; benzoxazol-5-yl-6-yl; benzoxazol-5-yl-7-yl; benzoxazol-6-yl-7-yl; benzoxadiazolyl; benzoxadiazol-2-yl; benzoxadiazol-3-yl; benzoxadiazol-4-yl; benzoxadiazol-5-yl; benzoxadiazol-6-yl; benzoxadiazol-7-yl; benzoxadiazolylyl; benzoxadiazol-2-yl-3-yl; benzoxadiazol-2-yl-4-yl; benzoxadiazol-2-yl-5-yl; benzoxadiazol-2-yl-6-yl; benzoxadiazol-2-yl-7-yl; benzoxadiazol-3-yl-4-yl; benzoxadiazol-3-yl-5-yl; benzoxadiazol-3-yl-6-yl; benzoxadiazol-3-yl-7-yl; benzoxadiazol-4-yl-5-yl; benzoxadiazol-4-yl-6-yl; benzoxadiazol-4-yl-7-yl; benzoxadiazol-5-yl-6-yl; benzoxadiazol-5-yl-7-yl; benzoxadiazol-6-yl-7-yl; benzothienyl; benzothien-1-yl; benzothien-2-yl; benzothien-3-yl; benzothien-4-yl; benzothien-5-yl; benzothien-7-yl; benzothien-7-yl; benzothienylyl; benzothien-1-yl-2-yl; benzothien-1-yl-3-yl; benzothien-1-yl-4-yl; benzothien-1-yl-5-yl; benzothien-1-yl-6-yl; benzothien-1-yl-7-yl; benzothien-2-yl-3-yl; benzothien-2-yl-4-yl; benzothien-2-yl-5-yl; benzothien-2-yl-6-yl; benzothien-2-yl-7-yl; benzothien-3-yl-4-yl; benzothien-3-yl-5-yl; benzothien-3-yl-6-yl; benzothien-3-yl-7-yl; benzothien-4-yl-5-yl; benzothien-4-yl-6-yl; benzothien-4-yl-7-yl; benzothien-5-yl-6-yl; benzothien-5-yl-7-yl; benzothien-6-yl-7-yl; quinolinyl; quinolin-1-yl; quinolin-2-yl; quinolin-3-yl; quinolin-4-yl; quinolin-5-yl; quinolin-6-yl; quinolin-7-yl; quinolin-8-yl; quinolinylyl; quinolin-1-yl-2-yl; quinolin-1-yl-3-yl; quinolin-1-yl-4-yl; quinolin-1-yl-5-yl; quinolin-1-yl-6-yl; quinolin-1-yl-7-yl; quinolin-1-yl-8-yl; quinolin-2-yl-3-yl; quinolin-2-yl-4-yl; quinolin-2-yl-5-yl; quinolin-2-yl-6-yl; quinolin-2-yl-7-yl; quinolin-2-yl-8-yl; quinolin-3-yl-4-yl; quinolin-3-yl-5-yl; quinolin-3-yl-6-yl; quinolin-3-yl-7-yl; quinolin-3-yl-8-yl; quinolin-4-yl-5-yl; quinolin-4-yl-6-yl; quinolin-4-yl-7-yl; quinolin-4-yl-8-yl; quinolin-5-yl-6-yl; quinolin-5-yl-7-yl; quinolin-5-yl-8-yl; quinolin-6-yl-7-yl; quinolin-6-yl-8-yl; quinolin-7-yl-8-yl; chromenyl; chromen-2-yl; chromen-3-yl; chromen-4-yl; chromen-5-yl; chromen-6-yl; chromen-7-yl; chromen-8-yl; chromenylyl; chromen-2-yl-3-yl; chromen-2-yl-4-yl; chromen-2-yl-5-yl; chromen-2-yl-6-yl; chromen-2-yl-7-yl; chromen-2-yl-8-yl; chromen-3-yl-4-yl; chromen-3-yl-5-yl; chromen-3-yl-6-yl; chromen-3-yl-7-yl; chromen-3-yl-8-yl; chromen-4-yl-5-yl; chromen-4-yl-6-yl; chromen-4-yl-7-yl; chromen-4-yl-8-yl; chromen-5-yl-6-yl; chromen-5-yl-7-yl; chromen-5-yl-8-yl; chromen-6-yl-7-yl; chromen-6-yl-8-yl; chromen-7-yl-8-yl; indolyl; indol-1-yl; indol-2-yl; indol-3-yl; indol-4-yl; indol-5-yl; indol-6-yl; indol-7-yl; indolylyl; indol-1-yl-2-yl; indol-1-yl-3-yl; indol-1-yl-4-yl; indol-1-yl-5-yl; indol-1-yl-6-yl; indol-1-yl-7-yl; indol-2-yl-3-yl; indol-2-yl-4-yl; indol-2-yl-5-yl; indol-2-yl-6-yl; indol-2-yl-7-yl; indol-3-yl-4-yl; indol-3-yl-5-yl; indol-3-yl-6-yl; indol-3-yl-7-yl; indol-4-yl-5-yl; indol-4-yl-6-yl; indol-4-yl-7-yl; indol-5-yl-6-yl; indol-5-yl-7-yl; indol-6-yl-7-yl; indazolyl; indazol-1-yl; indazol-2-yl; indazol-3-yl; indazol-4-yl; indazol-5-yl; indazol-6-yl; indazol-7-yl; indazolylyl; indazol-1-yl-2-yl; indazol-1-yl-3-yl; indazol-1-yl-4-yl; indazol-1-yl-5-yl; indazol-1-yl-6-yl; indazol-1-yl-7-yl; indazol-2-yl-3-yl; indazol-2-yl-4-yl; indazol-2-yl-5-yl; indazol-2-yl-6-yl; indazol-2-yl-7-yl; indazol-3-yl-4-yl; indazol-3-yl-5-yl; indazol-3-yl-6-yl; indazol-3-yl-7-yl; indazol-4-yl-5-yl; indazol-4-yl-6-yl; indazol-4-yl-7-yl; indazol-5-yl-6-yl; indazol-5-yl-7-yl; indazol-6-yl-7-yl; isoquinolinyl; isoquinolin-1-yl; isoquinolin-2-yl; isoquinolin-3-yl; isoquinolin-4-yl; isoquinolin-5-yl; isoquinolin-6-yl; isoquinolin-7-yl; isoquinolin-8-yl; isoquinolinylyl; isoquinolin-1-yl-2-yl; isoquinolin-1-yl-3-yl; isoquinolin-1-yl-4-yl; benzimidazolyl; isoquinolin-1-yl-5-yl; isoquinolin-1-yl-6-yl; isoquinolin-1-yl-7-yl; isoquinolin-1-yl-8-yl; isoquinolin-2-yl-3-yl; isoquinolin-2-yl-4-yl; isoquinolin-2-yl-5-yl; isoquinolin-2-yl-6-yl; isoquinolin-2-yl-7-yl; isoquinolin-2-yl-8-yl; isoquinolin-3-yl-4-yl; isoquinolin-3-yl-5-yl; isoquinolin-3-yl-6-yl; isoquinolin-3-yl-7-yl; isoquinolin-3-yl-8-yl; isoquinolin-4-yl-5-yl; isoquinolin-4-yl-6-yl; isoquinolin-4-yl-7-yl; isoquinolin-4-yl-8-yl; isoquinolin-5-yl-6-yl; isoquinolin-5-yl-7-yl; isoquinolin-5-yl-8-yl; isoquinolin-6-yl-7-yl; isoquinolin-6-yl-8-yl; isoquinolin-7-yl-8-yl; benzimidazolyl; benzimidazol-1-yl; benzimidazol-2-yl; benzimidazol-3-yl; benzimidazol-4-yl; benzimidazol-5-yl; benzimidazol-6-yl; benzimidazol-7-yl; benzimidazolylyl; benzimidazol-1-yl-2-yl; benzimidazol-1-yl-3-yl; benzimidazol-1-yl-4-yl; benzimidazol-1-yl-5-yl; benzimidazol-1-yl-6-yl; benzimidazol-1-yl-7-yl; benzimidazol-2-yl-3-yl; benzimidazol-2-yl-4-yl; benzimidazol-2-yl-5-yl; benzimidazol-2-yl-6-yl; benzimidazol-2-yl-7-yl; benzimidazol-3-yl-4-yl; benzimidazol-3-yl-5-yl; benzimidazol-3-yl-6-yl; benzimidazol-3-yl-7-yl; benzimidazol-4-yl-5-yl; benzimidazol-4-yl-6-yl; benzimidazol-4-yl-7-yl; benzimidazol-5-yl-6-yl; benzimidazol-5-yl-7-yl; benzimidazol-6-yl-7-yl; benzopyranyl; benzopyran-2-yl; benzopyran-3-yl; benzopyran-4-yl; benzopyran-5-yl; benzopyran-6-yl; benzopyran-7-yl; benzopyran-8-yl; benzopyranylyl; benzopyran-2-yl-3-yl; benzopyran-2-yl-4-yl; benzopyran-2-yl-5-yl; benzopyran-2-yl-6-yl; benzopyran-2-yl-7-yl; benzopyran-2-yl-8-yl; benzopyran-3-yl-4-yl; benzopyran-3-yl-5-yl; benzopyran-3-yl-6-yl; benzopyran-3-yl-7-yl; benzopyran-3-yl-8-yl; benzopyran-4-yl-5-yl; benzopyran-4-yl-6-yl; benzopyran-4-yl-7-yl; benzopyran-4-yl-8-yl; benzopyran-5-yl-6-yl; benzopyran-5-yl-7-yl; benzopyran-5-yl-8-yl; benzopyran-6-yl-7-yl; benzopyran-6-yl-8-yl; benzopyran-7-yl-8-yl; benzofuryl; benzofur-2-yl; benzofur-3-yl; benzofur-4-yl; benzofur-5-yl; benzofur-6-yl; benzofur-7-yl; benzofurylyl; benzofur-2-yl-3-yl; benzofur-2-yl-4-yl; benzofur-2-yl-5-yl; benzofur-2-yl-6-yl; benzofur-2-yl-7-yl; benzofur-3-yl-4-yl; benzofur-3-yl-5-yl; benzofur-3-yl-6-yl; benzofur-3-yl-7-yl; benzofur-4-yl-5-yl; benzofur-4-yl-6-yl; benzofur-4-yl-7-yl; benzofur-5-yl-6-yl; benzofur-5-yl-7-yl; benzofur-6-yl-7-yl; benzofurazanyl; benzofurazan-1-yl; benzofurazan-3-yl; benzofurazan-4-yl; benzofurazan-5-yl; benzofurazan-6-yl; benzofurazan-7-yl; benzofuranzanylyl; benzofurazan-1-yl-3-yl; benzofurazan-1-yl-4-yl; benzofurazan-1-yl-5-yl; benzofurazan-1-yl-6-yl; benzofurazan-1-yl-7-yl; benzofurazan-3-yl-4-yl; benzofurazan-3-yl-5-yl; benzofurazan-3-yl-6-yl; benzofurazan-3-yl-7-yl; benzofurazan-4-yl-5-yl; benzofurazan-4-yl-6-yl; benzofurazan-4-yl-7-yl; benzofurazan-5-yl-6-yl; benzofurazan-5-yl-7-yl; benzofurazan-6-yl-7-yl; benzopyranyl; benzopyran-2-yl; benzopyran-3-yl; benzopyran-4-yl; benzopyran-5-yl; benzopyran-6-yl; benzopyran-7-yl; benzopyran-8-yl; benzopyranylyl; benzopyran-2-yl-3-yl; benzopyran-2-yl-4-yl; benzopyran-2-yl-5-yl; benzopyran-2-yl-6-yl; benzopyran-2-yl-7-yl; benzopyran-2-yl-8-yl; benzopyran-3-yl-4-yl; benzopyran-3-yl-5-yl; benzopyran-3-yl-6-yl; benzopyran-3-yl-7-yl; benzopyran-3-yl-8-yl; benzopyran-4-yl-5-yl; benzopyran-4-yl-6-yl; benzopyran-4-yl-7-yl; benzopyran-4-yl-8-yl; benzopyran-5-yl-6-yl; benzopyran-5-yl-7-yl; benzopyran-5-yl-8-yl; benzopyran-6-yl-7-yl; benzopyran-6-yl-8-yl; benzopyran-7-yl-8-yl; cinnolinyl; cinnolin-1-yl; cinnolin-2-yl; cinnolin-3-yl; cinnolin-4-yl; cinnolin-5-yl; cinnolin-6-yl; cinnolin-7-yl; cinnolin-8-yl; cinnolinylyl; cinnolin-1-yl-2-yl; cinnolin-1-yl-3-yl; cinnolin-1-yl-4-yl; cinnolin-1-yl-5-yl; cinnolin-1-yl-6-yl; cinnolin-1-yl-7-yl; cinnolin-1-yl-8-yl; cinnolin-2-yl-3-yl; cinnolin-2-yl-4-yl; cinnolin-2-yl-5-yl; cinnolin-2-yl-6-yl; cinnolin-2-yl-7-yl; cinnolin-2-yl-8-yl; cinnolin-3-yl-4-yl; cinnolin-3-yl-5-yl; cinnolin-3-yl-6-yl; cinnolin-3-yl-7-yl; cinnolin-3-yl-8-yl; cinnolin-4-yl-5-yl; cinnolin-4-yl-6-yl; cinnolin-4-yl-7-yl; cinnolin-4-yl-8-yl; cinnolin-5-yl-6-yl; cinnolin-5-yl-7-yl; cinnolin-5-yl-8-yl; cinnolin-6-yl-7-yl; cinnolin-6-yl-8-yl; cinnolin-7-yl-8-yl; quinoxalinyl; quinoxalin-1-yl; quinoxalin-2-yl; quinoxalin-3-yl; quinoxalin-4-yl; quinoxalin-5-yl; quinoxalin-6-yl; quinoxalin-7-yl; quinoxalin-8-yl; quinoxalinylyl; quinoxalin-1-yl-2-yl; quinoxalin-1-yl-3-yl; quinoxalin-1-yl-4-yl; quinoxalin-1-yl-5-yl; quinoxalin-1-yl-6-yl; quinoxalin-1-yl-7-yl; quinoxalin-1-yl-8-yl; quinoxalin-2-yl-3-yl; quinoxalin-2-yl-4-yl; quinoxalin-2-yl-5-yl; quinoxalin-2-yl-6-yl; quinoxalin-2-yl-7-yl; quinoxalin-2-yl-8-yl; quinoxalin-3-yl-4-yl; quinoxalin-3-yl-5-yl; quinoxalin-3-yl-6-yl; quinoxalin-3-yl-7-yl; quinoxalin-3-yl-8-yl; quinoxalin-4-yl-5-yl; quinoxalin-4-yl-6-yl; quinoxalin-4-yl-7-yl; quinoxalin-4-yl-8-yl; quinoxalin-5-yl-6-yl; quinoxalin-5-yl-7-yl; quinoxalin-5-yl-8-yl; quinoxalin-6-yl-7-yl; quinoxalin-6-yl-8-yl; and quinoxalin-7-yl-8-yl. Unless reference is made to a specific point of attachment, e.g. as in indol-4-yl, indol-5-yl-6-yl, it is intended that such heteroaryl groups can be bonded to at least one other moiety at any available point of attachment.

The term “cycloalkyl” refers to a fully saturated and partially unsaturated cyclic hydrocarbon group containing from 1 to 3 rings and 3 to 8 carbons per ring. Exemplary cycloalkyls include, but are not limited to, for example, cyclopropyl; cyclopropylyl; cycloprop-1-yl-2-yl; cyclobutyl; cyclobutylyl; cyclobut-1-yl-2-yl; cyclobut-1-yl-3-yl; cyclopentyl; cyclopentylyl; cyclopent-1-yl-2-yl; cyclopent-1-yl-3-yl; cyclohexyl; cyclohexylyl; cyclohex-1-yl-2-yl; cyclohex-1-yl-3-yl; cyclohex-1-yl-4-yl; cycloheptyl; cycloheptylyl; cyclohept-1-yl-2-yl; cyclohept-1-yl-3-yl; cyclohept-1-yl-4-yl; cyclooctyl; cyclooct-1-yl-2-yl; cyclooct-1-yl-3-yl; cyclooct-1-yl-4-yl; cyclooct-1-yl-5-yl; cyclobutenyl; cyclobuten-1-yl; cyclobuten-2-yl; cyclobuten-3-yl; cyclobuten-4-yl; cyclobutenylyl; cyclobuten-1-yl-2-yl; cyclobuten-1-yl-3-yl; cyclobuten-1-yl-4-yl; cyclobuten-2-yl-3-yl; cyclobuten-2-yl-4-yl; cyclobuten-3-yl-4-yl; cyclopentenyl; cyclopenten-1-yl; cyclopenten-2-yl; cyclopenten-3-yl; cyclopenten-4-yl; cyclopenten-5-yl; cyclopentenylyl; cyclopenten-1-yl-2-yl; cyclopenten-1-yl-3-yl; cyclopenten-1-yl-4-yl; cyclopenten-1-yl-5-yl; cyclopenten-2-yl-3-yl; cyclopenten-2-yl-4-yl; cyclopenten-2-yl-5-yl; cyclopenten-3-yl-4-yl; cyclopenten-3-yl-5-yl; cyclopenten-4-yl-5-yl; cyclohexenyl; cyclohexen-1-yl; cyclohexen-2-yl; cyclohexen-3-yl; cyclohexen-4-yl; cyclohexen-5-yl; cyclohexen-6-yl; cyclohexenylyl; cyclohexen-1-yl-2-yl; cyclohexen-1-yl-3-yl; cyclohexen-1-yl-4-yl; cyclohexen-1-yl-5-yl; cyclohexen-1-yl-6-yl; cyclohexen-2-yl-3-yl; cyclohexen-2-yl-4-yl; cyclohexen-2-yl-5-yl; cyclohexen-2-yl-6-yl; cyclohexen-3-yl-4-yl; cyclohexen-3-yl-5-yl; cyclohexen-3-yl-6-yl; cyclohexen-4-yl-5-yl; cyclohexen-4-yl-6-yl; and cyclohexen-5-yl-6-yl. A cycloalkyl ring may have a carbon ring atom replaced with a carbonyl group (C═O). Cycloalkyls include rings having a second or third ring fused thereto that is a heterocyclo, heteroaryl, or aryl, provided that in such cases the point of attachment is to the cycloalkyl portion of the ring system. The term “cycloalkyl” also includes rings having a second or third ring attached to the ring or ring system in a spiro fashion. Unless reference is made to a specific point of attachment, e.g. as in cyclohexen-3-yl-6-yl, cycloprop-1-yl-2-yl, and cyclobuten-4-yl, it is intended that such cycloalkyl groups can be bonded to at least one other moiety at any available point of attachment.

The term “heterocycloalkyl” refers to a saturated or unsaturated cycloalkyl in which at least one ring carbon (and any associated hydrogen atoms) are independently replaced with at least one heteroatom selected from O and N.

The terms “halogen” and “halo” refer to chlorine, bromine, fluorine, and iodine.

The term “haloalkyl” refers to an alkyl bonded to a single halogen or multiple halogens. Exemplary haloalkyls containing multiple halogens include, but are not limited to, for example, —CHCl₂ and —CF₃.

The term “alkoxy” used alone or as a suffix or prefix, refers to radicals of the general formula —OR^{a}, wherein R^{a} is selected from a hydrocarbon radical. Exemplary alkoxys include, but are not limited to, for example, methoxy, ethoxy, propoxy, isopropoxy, butoxy, t-butoxy, isobutoxy, cyclopropylmethoxy, allyloxy, and propargyloxy.

The term “cyano” refers to CN.

The phrase “optionally substituted” refers to either groups, structures, or molecules that are substituted with at least one substituent at any available and substitutable position and groups, structures, or molecules that are not substituted.

The phrase “a compound of formula I, or pharmaceutically acceptable salts of formula I, or mixtures thereof” refers to the free base of formula I, pharmaceutically acceptable salts of formula I, and/or mixtures of any of the foregoing.

In one aspect, the disclosure is directed to a compound of formula I, or pharmaceutically acceptable salts of formula I, or mixtures thereof:

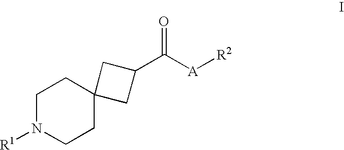

wherein

A is

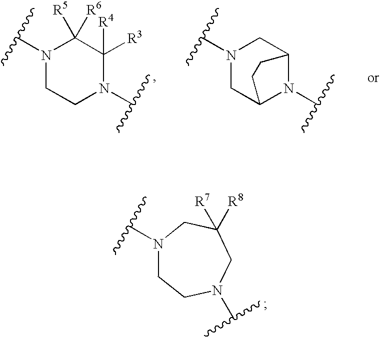

R¹ is aryl, 5- or 6-membered heteroaryl, —S(═O)₂R⁹, —C(═O)R¹⁰, or —C(═O)NR¹¹R¹²;

R² is C₃₋₆cycloalkyl or C₁₋₆alkyl;

R³, R⁴, R⁵, R⁶, R², and R⁸ are each, independently, selected from hydrogen and —C₁-C₃alkyl;

R⁹, R¹⁰, and R¹¹ are each, independently, selected from C₁₋₆alkyl, 4-membered heterocycloalkyl, 5-membered heterocycloalkyl, 6-membered heterocycloalkyl, 7-membered heterocycloalkyl, C₃₋₇cycloalkyl, aryl, 5-membered heteroaryl, 6-membered heteroaryl, (C₁₋₃alkyl)-(5-membered heteroaryl), and (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;

R¹² is H or C₁₋₆alkyl; and

R¹³ and R¹⁴ are each, independently, selected from H and C₁₋₃alkyl.

In another aspect, the disclosure is directed to a compound of formula I, or pharmaceutically acceptable salts of formula I, or mixtures thereof:

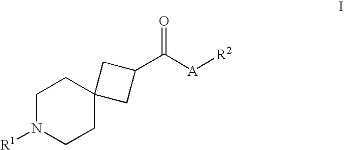

wherein

A is

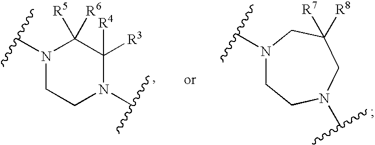

R¹ is aryl, 5- or 6-membered heteroaryl, —S(═O)₂R⁹, —C(═O)R¹⁶, or —C(═O)NR¹¹R¹²;

R² is C₃₋₆cycloalkyl or C₁₋₆alkyl;

R³, R⁴, R⁵, R⁶, R⁷, and R⁸ are each, independently, selected from hydrogen and —C₁-C₃alkyl;

R⁹ is C₁₋₆alkyl or aryl, wherein said aryl is optionally substituted by 1, 2, or 3 substituents selected from halo;

R¹⁰ is C₃₋₆cycloalkyl, aryl, 5-membered heteroaryl, 6-membered heteroaryl, —(C₁₋₃alkyl)-(5-membered heteroaryl), or (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;

R¹¹ and R¹² are each, independently, selected from H, aryl, and C₁₋₃alkyl; and

R¹³ and R¹⁴ are each, independently, selected from H and C₁₋₃alkyl.

In another embodiment formula I is

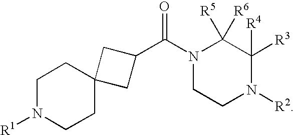

In yet another embodiment, formula I is

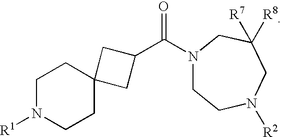

In still yet another embodiment, formula I is

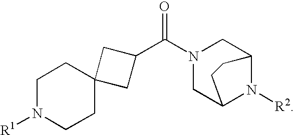

In one embodiment, A is

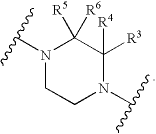

In another embodiment, A is

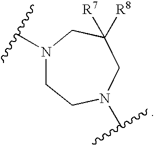

In an even further embodiment, A is piperazin-1-yl-4-yl.

In yet an even further embodiment, A is 1,4-diazepan-1-yl-4-yl.

In yet a further embodiment, R¹ is phenyl, 6-membered heteroaryl, —S(═O)₂R⁹, —C(═O)R¹⁰, or —C(═O)NR¹¹R¹².

In a further embodiment, R¹ is phenyl, pyridinyl, pyrazinyl, pyrimidinyl, —S(═O)₂R⁹, —C(═O)R¹⁰, or —C(═O)NR¹¹R¹².

In still yet a further embodiment, R⁹ is C₁₋₃alkyl or phenyl.

In a still further embodiment, R⁹ is an aryl substituted by 1, 2, or 3 substituents selected from halo.

In yet another embodiment, R¹⁰ is C₃₋₆cycloalkyl, aryl, 6-membered heteroaryl optionally substituted with cyano, or (C₁₋₃alkyl)-(6-membered heteroaryl).

In still yet a further embodiment, R¹⁰ is cyclohexyl, pyridinyl, phenyl, nicotinonitrile, methylpyridine, or ethylpyridine.

In still another embodiment, R¹⁰ is an aryl or 6-membered heteroaryl, wherein said aryl or heteroaryl are each, independently, substituted by 1 or 2 substituents selected from halo, cyano, C₁₋₃alkyl, and C₁₋₃alkoxy.

In yet another embodiment, R¹⁰ is an aryl substituted by 1 or 2 substituents selected from cyano.

In still yet another embodiment, R¹⁰ is a 6-membered heteroaryl substituted by 1 or 2 substituents selected from halo, cyano, C₁₋₃alkyl, and C₁₋₃alkoxy.

In yet a further embodiment, R¹⁰ is a 6-membered heteroaryl substituted by 1 or 2 substituents selected from methyl, methoxy, ethyl, cyano, fluoro, and chloro.

In an even further embodiment, R¹¹ and R¹² are each, independently, selected from H, phenyl, and C₁₋₃alkyl.

In still another embodiment, R³, R⁴, R⁵, R⁶, R⁷, and R⁸ are each, independently, hydrogen.

In yet still another embodiment, R³, R⁴, R⁵, R⁶, R⁷, and R⁸ are each, independently, hydrogen or CH₃.

Yet an even further embodiment is directed to at least one compound selected from (4-cyclobutylpiperazin-1-yl)(7-(pyridin-3-yl)-7-azaspiro[3.5]nonan-2-yl)methanone; (4-cyclobutylpiperazin-1-yl)(7-(pyridin-2-yl)-7-azaspiro[3.5]nonan-2-yl)methanone; (4-cyclobutylpiperazin-1-yl)(7-phenyl-7-azaspiro[3.5]nonan-2-yl)methanone; (4-cyclobutylpiperazin-1-yl)(7-(isopropylsulfonyl)-7-azaspiro[3.5]nonan-2-yl)methanone; (4-cyclobutylpiperazin-1-yl)(7-(methylsulfonyl)-7-azaspiro[3.5]nonan-2-yl)methanone; (4-cyclobutylpiperazin-1-yl)(7-(phenylsulfonyl)-7-azaspiro[3.5]nonan-2-yl)methanone; (4-cyclobutylpiperazin-1-yl)(7-(cyclohexanecarbonyl)-7-azaspiro[3.5]nonan-2-yl)methanone; (4-cyclobutylpiperazin-1-yl)(7-(3-ethylisonicotinoyl)-7-azaspiro[3.5]nonan-2-yl)methanone; (7-(5-chloronicotinoyl)-7-azaspiro[3.5]nonan-2-yl)(4-cyclobutylpiperazin-1-yl)methanone; (7-(4-chloronicotinoyl)-7-azaspiro[3.5]nonan-2-yl)(4-cyclobutylpiperazin-1-yl)methanone; (4-cyclobutylpiperazin-1-yl)(7-(2,4-dimethylnicotinoyl)-7-azaspiro[3.5]nonan-2-yl)methanone; (4-cyclobutylpiperazin-1-yl)(7-(6-methylnicotinoyl)-7-azaspiro[3.5]nonan-2-yl)methanone; (4-cyclobutylpiperazin-1-yl)(7-(2-methylnicotinoyl)-7-azaspiro[3.5]nonan-2-yl)methanone; (4-cyclobutylpiperazin-1-yl)(7-(4-methylnicotinoyl)-7-azaspiro[3.5]nonan-2-yl)methanone; 6-(2-(4-cyclobutylpiperazine-1-carbonyl)-7-azaspiro[3.5]nonane-7-carbonyl)nicotinonitrile; (4-cyclobutylpiperazin-1-yl)(7-(3,5-difluoropicolinoyl)-7-azaspiro[3.5]nonan-2-yl)methanone; (7-(4-chloropicolinoyl)-7-azaspiro[3.5]nonan-2-yl)(4-cyclobutylpiperazin-1-yl)methanone; (4-cyclobutylpiperazin-1-yl)(7-(6-methylpicolinoyl)-7-azaspiro[3.5]nonan-2-yl)methanone; (4-cyclobutylpiperazin-1-yl)(7-(3-methylpicolinoyl)-7-azaspiro[3.5]nonan-2-yl)methanone; (4-cyclobutylpiperazin-1-yl)(7-picolinoyl-7-azaspiro[3.5]nonan-2-yl)methanone; (4-cyclobutylpiperazin-1-yl)(7-nicotinoyl-7-azaspiro[3.5]nonan-2-yl)methanone; (4-cyclobutylpiperazin-1-yl)(7-isonicotinoyl-7-azaspiro[3.5]nonan-2-yl)methanone; (4-cyclobutylpiperazin-1-yl)(7-(3-methylisonicotinoyl)-7-azaspiro[3.5]nonan-2-yl)methanone; (4-cyclobutylpiperazin-1-yl)(7-(2-methylisonicotinoyl)-7-azaspiro[3.5]nonan-2-yl)methanone; (4-cyclobutylpiperazin-1-yl)(7-(5-methylnicotinoyl)-7-azaspiro[3.5]nonan-2-yl)methanone; (4-cyclobutylpiperazin-1-yl)(7-(2-methoxyisonicotinoyl)-7-azaspiro[3.5]nonan-2-yl)methanone; 1-(2-(4-cyclobutylpiperazine-1-carbonyl)-7-azaspiro[3.5]nonan-7-yl)-2-(pyridin-3-yl)ethanone; 1-(2-(4-cyclobutylpiperazine-1-carbonyl)-7-azaspiro[3.5]nonan-7-yl)-2-(pyridin-4-yl)ethanone; 1-(2-(4-cyclobutylpiperazine-1-carbonyl)-7-azaspiro[3.5]nonan-7-yl)-3-(pyridin-4-yl)propan-1-one; (7-benzoyl-7-azaspiro[3.5]nonan-2-yl)(4-cyclobutylpiperazin-1-yl)methanone; 3-(2-(4-cyclobutylpiperazine-1-carbonyl)-7-azaspiro[3.5]nonane-7-carbonyl)benzonitrile; 2-(4-cyclobutylpiperazine-1-carbonyl)-N-phenyl-7-azaspiro[3.5]nonane-7-carboxamide; (4-isopropylpiperazin-1-yl)(7-(methylsulfonyl)-7-azaspiro[3.5]nonan-2-yl)methanone; (4-isopropylpiperazin-1-yl)(7-(2-methylisonicotinoyl)-7-azaspiro[3.5]nonan-2-yl)methanone; (7-isonicotinoyl-7-azaspiro[3.5]nonan-2-yl)(4-isopropylpiperazin-1-yl)methanone; (4-isopropylpiperazin-1-yl)(7-nicotinoyl-7-azaspiro[3.5]nonan-2-yl)methanone; 2-(4-isopropylpiperazine-1-carbonyl)-N,N-dimethyl-7-azaspiro[3.5]nonane-7-carboxamide; (4-cyclobutyl-1,4-diazepan-1-yl)(7-(4-fluorophenylsulfonyl)-7-azaspiro[3.5]nonan-2-yl)methanone; (4-cyclobutyl-1,4-diazepan-1-yl)(7-(3-fluorophenylsulfonyl)-7-azaspiro[3.5]nonan-2-yl)methanone; (4-cyclobutyl-1,4-diazepan-1-yl)(7-(2-fluorophenylsulfonyl)-7-azaspiro[3.5]nonan-2-yl)methanone; (4-cyclobutyl-1,4-diazepan-1-yl)(7-(phenylsulfonyl)-7-azaspiro[3.5]nonan-2-yl)methanone; (4-cyclobutyl-1,4-diazepan-1-yl)(7-picolinoyl-7-azaspiro[3.5]nonan-2-yl)methanone; (4-cyclobutyl-1,4-diazepan-1-yl)(7-(2-methylisonicotinoyl)-7-azaspiro[3.5]nonan-2-yl)methanone; (4-cyclobutyl-1,4-diazepan-1-yl)(7-(3-methylisonicotinoyl)-7-azaspiro[3.5]nonan-2-yl)methanone; (4-cyclobutyl-1,4-diazepan-1-yl)(7-isonicotinoyl-7-azaspiro[3.5]nonan-2-yl)methanone; (4-cyclobutyl-1,4-diazepan-1-yl)(7-(5-methylnicotinoyl)-7-azaspiro[3.5]nonan-2-yl)methanone; (4-cyclobutyl-1,4-diazepan-1-yl)(7-nicotinoyl-7-azaspiro[3.5]nonan-2-yl)methanone; (7-benzoyl-7-azaspiro[3.5]nonan-2-yl)(4-cyclobutyl-1,4-diazepan-1-yl)methanone; 4-(2-(4-cyclobutyl-1,4-diazepane-1-carbonyl)-7-azaspiro[3.5]nonane-7-carbonyl)benzonitrile; 3-(2-(4-cyclobutyl-1,4-diazepane-1-carbonyl)-7-azaspiro[3.5]nonane-7-carbonyl)benzonitrile; 2-(2-(4-cyclobutyl-1,4-diazepane-1-carbonyl)-7-azaspiro[3.5]nonane-7-carbonyl)benzonitrile; (4-isopropyl-1,4-diazepan-1-yl)(7-(methylsulfonyl)-7-azaspiro[3.5]nonan-2-yl)methanone; (4-isopropyl-1,4-diazepan-1-yl)(7-(2-methylisonicotinoyl)-7-azaspiro[3.5]nonan-2-yl)methanone; (7-isonicotinoyl-7-azaspiro[3.5]nonan-2-yl)(4-isopropyl-1,4-diazepan-1-yl)methanone; (4-isopropyl-1,4-diazepan-1-yl)(7-nicotinoyl-7-azaspiro[3.5]nonan-2-yl)methanone; 2-(4-isopropyl-1,4-diazepane-1-carbonyl)-N,N-dimethyl-7-azaspiro[3.5]nonane-7-carboxamide; and (4-cyclobutyl-6,6-dimethyl-1,4-diazepan-1-yl)(7-(2-methylisonicotinoyl)-7-azaspiro[3.5]nonan-2-yl)methanone; (4-cyclopentylpiperazin-1-yl)(7-(methylsulfonyl)-7-azaspiro[3.5]nonan-2-yl)methanone; ((S)-4-isopropyl-3-methylpiperazin-1-yl)(7-(methylsulfonyl)-7-azaspiro[3.5]nonan-2-yl)methanone; ((R)-4-cyclobutyl-2-methylpiperazin-1-yl)(7-(methylsulfonyl)-7-azaspiro[3.5]nonan-2-yl)methanone; 4-(2-(4-cyclobutylpiperazine-1-carbonyl)-7-azaspiro[3.5]nonane-7-carbonyl)nicotinonitrile; (4-cyclobutylpiperazin-1-yl)(7-(pyrazin-2-yl)-7-azaspiro[3.5]nonan-2-yl)methanone; (4-cyclobutylpiperazin-1-yl)(7-(pyridin-4-yl)-7-azaspiro[3.5]nonan-2-yl)methanone; and (4-cyclobutylpiperazin-1-yl)(7-(pyrimidin-5-yl)-7-azaspiro[3.5]nonan-2-yl)methanone; and pharmaceutically acceptable salts thereof and mixtures thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of compounds in accordance with formula I. The optically active forms of compounds of the invention may be prepared, for example, by chiral chromatographic separation of a racemate, by synthesis from optically active starting materials or by asymmetric synthesis based on the procedures described hereafter.

It will be understood that the present invention encompasses any tautomers of compounds in accordance with formula I.

It will also be understood that certain compounds of the invention may exist in solvated, for example hydrated, as well as unsolvated forms. It will further be understood that the present invention encompasses all such solvated forms of compounds in accordance with formula I.

The compounds of formula I can also form salts. As a result, when a compound of formula I is referred to herein, such reference includes, unless otherwise indicated, salts thereof. In one embodiment, compounds of formula I form pharmaceutically acceptable salts. In another embodiment, compounds of formula I form salts that can, for example, be used to isolate and/or purify compounds in accordance with formula I.

Generally, pharmaceutically acceptable salts of a compound in accordance with formula I can be obtained by using standard procedures well known in the art. These standard procedures include, but are not limited to, for example, the reacting of a sufficiently basic compound, such as, for example, an alkyl amine with a suitable acid, such as, for example, HCl or acetic acid, to afford a physiologically acceptable anion. It may also be possible to make a corresponding alkali metal (such as sodium, potassium, or lithium) or an alkaline earth metal (such as a calcium) salt by treating a compound in accordance with formula I having a suitably acidic proton, such as, for example, a carboxylic acid or a phenol with one equivalent of an alkali metal or alkaline earth metal hydroxide or alkoxide (such as, for example, an ethoxide or methoxide), or a suitably basic organic amine (such as, for example, a choline or meglumine) in an aqueous medium, followed by conventional purification techniques.

In one embodiment, a compound in accordance with formula I may be converted to a pharmaceutically acceptable salt or solvate thereof, particularly, an acid addition salt, such as, for example, hydrochloride, hydrobromide, phosphate, acetate, fumarate, maleate, tartrate, citrate, methanesulphonate, and p-toluenesulphonate.

In general, compounds of formula I can be prepared in accordance with the following Schemes and the general knowledge of one skilled in the art and/or in accordance with the methods set forth in the Examples that follow. Solvents, temperatures, pressures, and other reaction conditions may readily be selected by one of ordinary skill in the art. Starting materials are commercially available or readily prepared by one skilled in the art. Combinatorial techniques can be employed in the preparation of compounds, for example, where the intermediates possess groups suitable for these techniques.

The term “amino-protecting group” refers to art-recognized moieties capable of attaching to an amino group so as to prevent the amino group from taking place in reactions occurring elsewhere on the molecule containing the amino group. Acceptable amino-protecting groups, include but are not limited to, for example, amino-protecting groups described in “Protective Groups in Organic Synthesis”, 2nd edition, John Wiley & Sons, 1991. The amino-protecting group may, for example, be a urethane type protective group (which is also referred to as a carbamate protective group), including but not limited to, for example, arylalkyloxycarbonyl groups, such as, for example, benzyloxycarbonyl; and alkoxycarbonyl groups, such as, for example, methoxycarbonyl and tert-butoxycarbonyl. Typically, the amino-protecting group is tert-butoxycarbonyl.

The term “carbonyl-protecting group” refers to art-recognized moieties capable of attaching to a carbonyl group so as to prevent the carbonyl group from taking place in reactions occurring elsewhere on the molecule containing the carbonyl group. Acceptable carbonyl-protecting groups, include but are not limited to, for example, carbonyl-protecting groups described in “Protective Groups in Organic Synthesis”, 2nd edition, John Wiley & Sons, 1991. The carbonyl-protecting group may, for example, be a cyclic ketal protective group (which is also referred to as a 1,3-dioxane, 1,3-dioxolane, 1,3-dithiane, and 1,3-dithiolane protective group), including but not limited to, for example, aliphatic cyclic ketones, such as, for example, 1,3-dioxane and 1,3-dioxolane. Typically, the carbonyl-protecting group is 1,3-dioxolane.

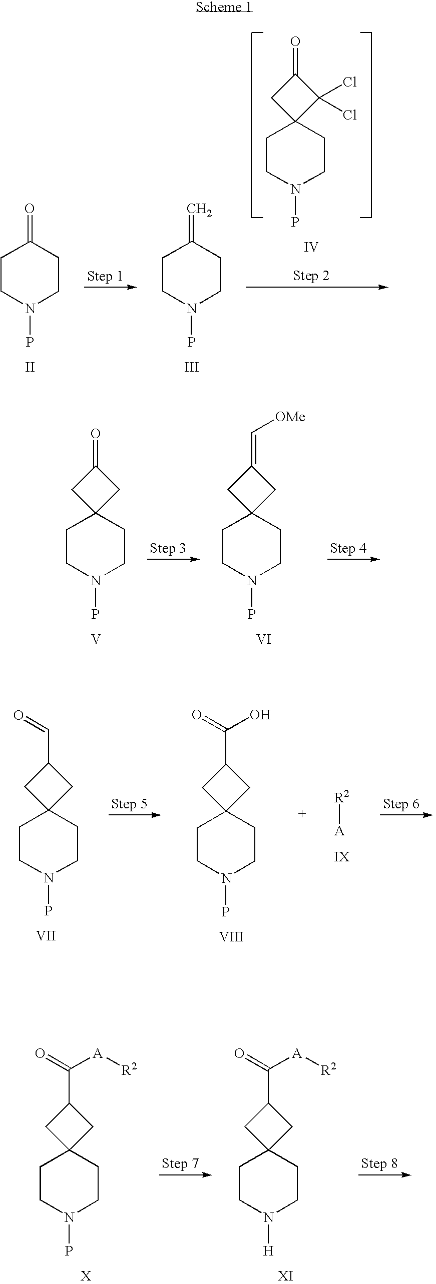

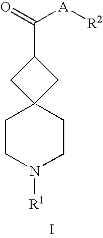

wherein P is an amino protecting group and A, R¹, and R² are as defined hereinabove.

### Step 1

A compound in accordance with formula III can be obtained by treating an appropriate ketone of a compound in accordance with formula II and an appropriate Wittig reagent such as, for example, methyltriphenylphosphonium bromide and an appropriate base, such as, for example, n-butyl lithium in an appropriate solvent, such as, for example, tetrahydrofuran.

### Step 2

A compound in accordance with formula V can be obtained by treating a compound in accordance with formula III with an appropriate cyclizing reagent, such as, for example, trichloroacetyl chloride in the presence of an appropriate metal catalyst, such as, for example, Zn—Cu couple in an appropriate solvent, such as, for example, methyl tert-butyl ether.

### Step 3

A compound in accordance with formula VI can be obtained by treating a compound in accordance with formula V with an appropriate Wittig reagent, such as, for example, Ph₃PCH₂(OMe)Cl and an appropriate base, such as, for example, potassium t-butoxide in an appropriate solvent, such as, for example a mix of tetrahydrofuran and t-butanol.

### Step 4

A compound in accordance with formula VII can be obtained by treating a compound in accordance with formula VI with an appropriate acid, such as, for example, aqueous hydrochloric acid in an appropriate solvent, such as, for example acetonitrile.

### Step 5

A compound in accordance with formula VIII can be obtained by treating a compound in accordance with formula VII with an appropriate oxidizing reagent, such as, for example, TEMPO and a bleach solution and an appropriate base, such as, for example, aqueous sodium bicarbonate in an appropriate solvent, such as, for example a mixture of dichloromethane and water

### Step 6

A compound in accordance with formula X can be obtained by the treating a compound in accordance with formula VIII with an appropriate coupling reagent, such as, for example, HBTU and an appropriate base, such as, for example, DIEA in an appropriate solvent, such as, for example DMF followed by treating with an appropriately functionalized compound in accordance with formula IX, which can prepared in accordance with Scheme 2 or is generally commercially available in an appropriate solvent, such as, for example, dimethylformamide. Commercially available formula IX compounds include, but are not limited to, for example, 1-isopropylpiperazine.

### Step 7

A compound in accordance with formula XI can be obtained by treating a compound in accordance with formula X with an appropriate catalyst, such as, for example, palladium on carbon in an appropriate solvent, such as, for example, ethanol in a atmosphere of hydrogen.

### Step 8

A compound in accordance with Formula I can be obtained by treating a compound in accordance with formula XI with an appropriate coupling reagent, such as, for example, HBTU and an appropriate base, such as, for example, DIEA in an appropriate solvent, such as, for example DMF.

Alternatively, a compound in accordance with Formula I can be obtained by treating a compound in accordance with formula XI with an appropriate sulfonyl chloride and an appropriate base, such as, for example, triethylamine in an appropriate solvent, such as, for example dichloromethane.

Alternatively, a compound in accordance with Formula I can be obtained by treating a compound in accordance with formula XI with an appropriate acid chloride and an appropriate base, such as, for example, DIEA in an appropriate solvent, such as, for example dichloromethane.

Alternatively, a compound in accordance with Formula I can be obtained by treating a compound in accordance with formula XI with an appropriate isocyanate, such as, for example, phenyl isocyanate in an appropriate solvent, such as, for example dichloromethane

Alternatively, a compound in accordance with Formula I can be obtained by treating a compound in accordance with formula XI with an appropriate catalyst, such as, for example palladium acetate and an appropriate ligand, such as, for example, BINAP and an appropriate aryl halide and an appropriate base, such as, for example, cesium carbonate in an appropriate solvent, such as, for example toluene.

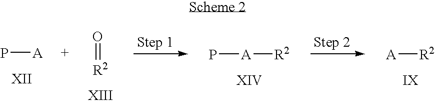

wherein P is an amino protecting group, and A and R² are as defined hereinabove.

### Step 1

A compound in accordance with formula XIV can be obtained by treating a compound in accordance with formula XII with an appropriately functionalized aldehyde or ketone, such as, for example, a compound in accordance with formula XIII, which is generally commercially available in the presence of an appropriate borohydride reagent, such as, for example, sodium triacetoxyborohydride in an appropriate solvent, such as, for example, dichloroethane.

### Step 2

A compound in accordance with formula IX can be obtained by treating a compound in accordance with formula XIV with an appropriate acid, such as, for example, hydrochloric acid in an appropriate solvent, such as, for example, ethyl acetate at reduced temperatures.

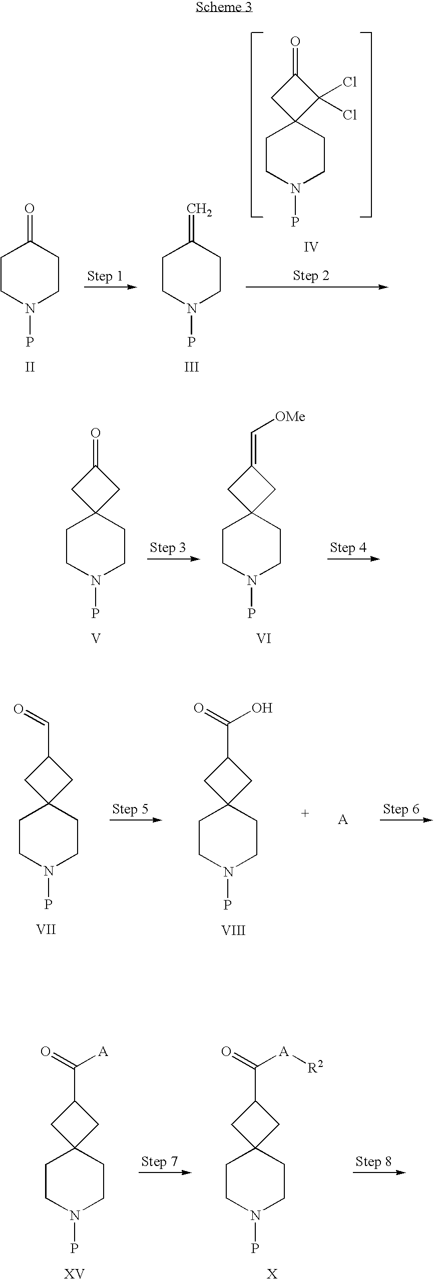

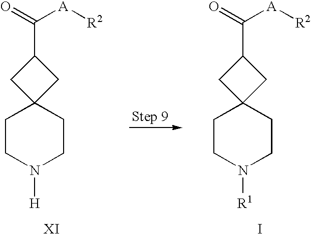

wherein P is an amino protecting group, A is

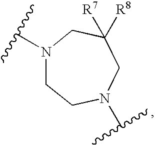

and R¹ and R² are as defined hereinabove.

### Step 1

A compound in accordance with formula III can be obtained by treating an appropriate ketone of a compound in accordance with formula II and an appropriate Wittig reagent such as, for example, methyltriphenylphosphonium bromide and an appropriate base, such as, for example, n-butyl lithium in an appropriate solvent, such as, for example, tetrahydrofuran.

### Step 2

A compound in accordance with formula V can be obtained by treating a compound in accordance with formula III with an appropriate cyclizing reagent, such as, for example, trichloroacetyl chloride in the presence of an appropriate metal catalyst, such as, for example, Zn—Cu couple in an appropriate solvent, such as, for example, methyl tert-butyl ether.

### Step 3

A compound in accordance with formula VI can be obtained by treating a compound in accordance with formula V with an appropriate Wittig reagent, such as, for example, Ph₃PCH₂(OMe)Cl and an appropriate base, such as, for example, potassium t-butoxide in an appropriate solvent, such as, for example a mix of tetrahydrofuran and t-butanol.

### Step 4

A compound in accordance with formula VII can be obtained by treating a compound in accordance with formula VI with an appropriate acid, such as, for example, aqueous hydrochloric acid in an appropriate solvent, such as, for example acetonitrile.

### Step 5

A compound in accordance with formula VIII can be obtained by treating a compound in accordance with formula VII with an appropriate oxidizing reagent, such as, for example, TEMPO and a bleach solution and an appropriate base, such as, for example, aqueous sodium bicarbonate in an appropriate solvent, such as, for example a mixture of dichloromethane and water

### Step 6

A compound in accordance with Formula XV can be obtained by treating a compound in accordance with formula VIII with oxalyl chloride, an A group and an appropriate base, such as, for example, triethylamine in an appropriate solvent, such as, for example dichloromethane.

### Step 7

A compound in accordance with Formula X can be obtained by treating a compound in accordance with formula XV with an appropriate reducing agent, such as, for example sodium triacetoxyborohydride and an appropriate ketone, such as, for example, cyclobutanone in an appropriate solvent, such as, for example ethanol.

### Step 8

A compound in accordance with formula XI can be obtained by treating a compound in accordance with formula X with an appropriate catalyst, such as, for example, palladium on carbon in an appropriate solvent, such as, for example, ethanol in a atmosphere of hydrogen.

### Step 9

A compound in accordance with Formula I can be obtained by treating a compound in accordance with formula XI with an appropriate coupling reagent, such as, for example, HBTU and an appropriate base, such as, for example, DIEA in an appropriate solvent, such as, for example DMF.

Alternatively, a compound in accordance with Formula I can be obtained by treating a compound in accordance with formula XI with an appropriate sulfonyl chloride and an appropriate base, such as, for example, triethylamine in an appropriate solvent, such as, for example dichloromethane.

Alternatively, a compound in accordance with Formula I can be obtained by treating a compound in accordance with formula XI with an appropriate acid chloride and an appropriate base, such as, for example, DIEA in an appropriate solvent, such as, for example dichloromethane.

Alternatively, a compound in accordance with Formula I can be obtained by treating a compound in accordance with formula XI with an appropriate isocyanate, such as, for example, phenyl isocyanate in an appropriate solvent, such as, for example dichloromethane

Alternatively, a compound in accordance with Formula I can be obtained by treating a compound in accordance with formula XI with an appropriate catalyst, such as, for example palladium acetate and an appropriate ligand, such as, for example, BINAP and an appropriate aryl halide and an appropriate base, such as, for example, cesium carbonate in an appropriate solvent, such as, for example toluene.

The solvents, temperatures, pressures, and other reaction conditions of Schemes 4-9 may be readily selected by one of ordinary skill in the art. Examples of the solvents, temperatures, pressures, and other reaction conditions that can be employed in Schemes 4-9 are identified in the Example section set forth hereinbelow.

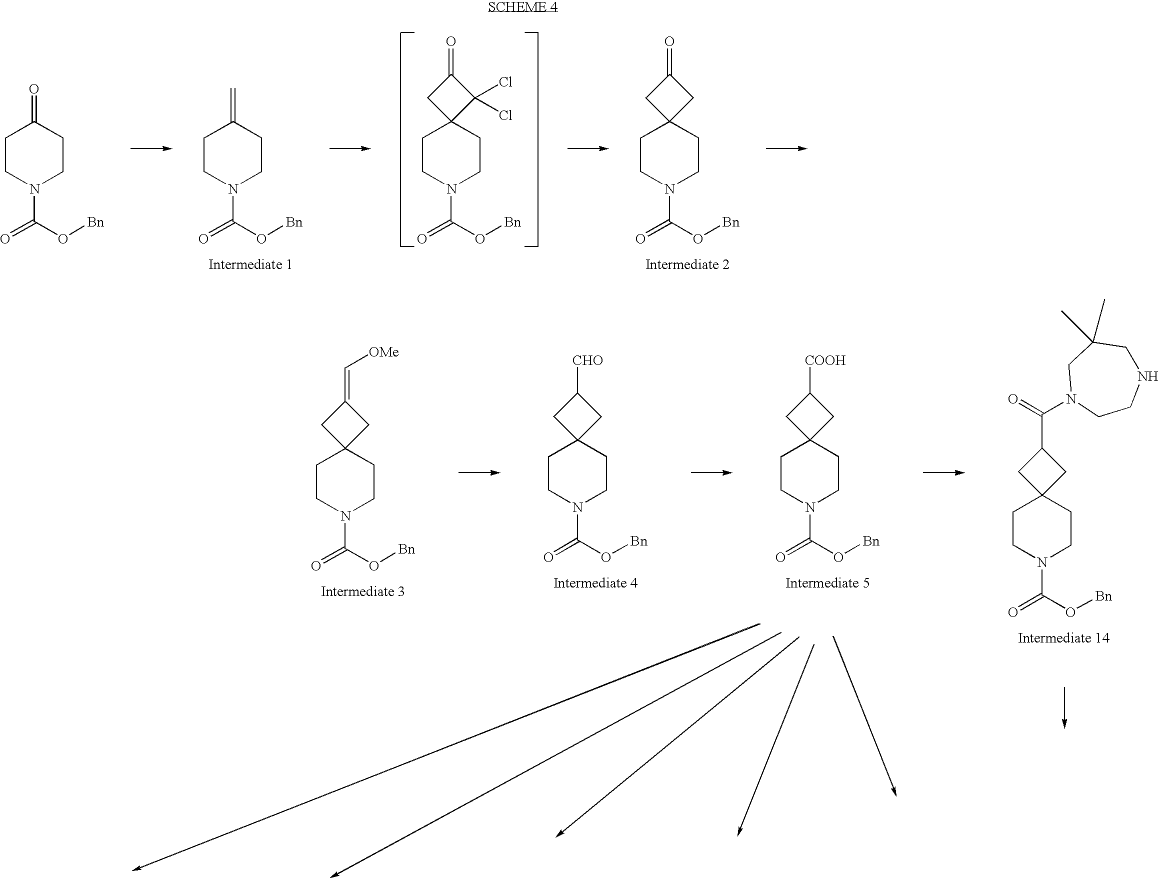

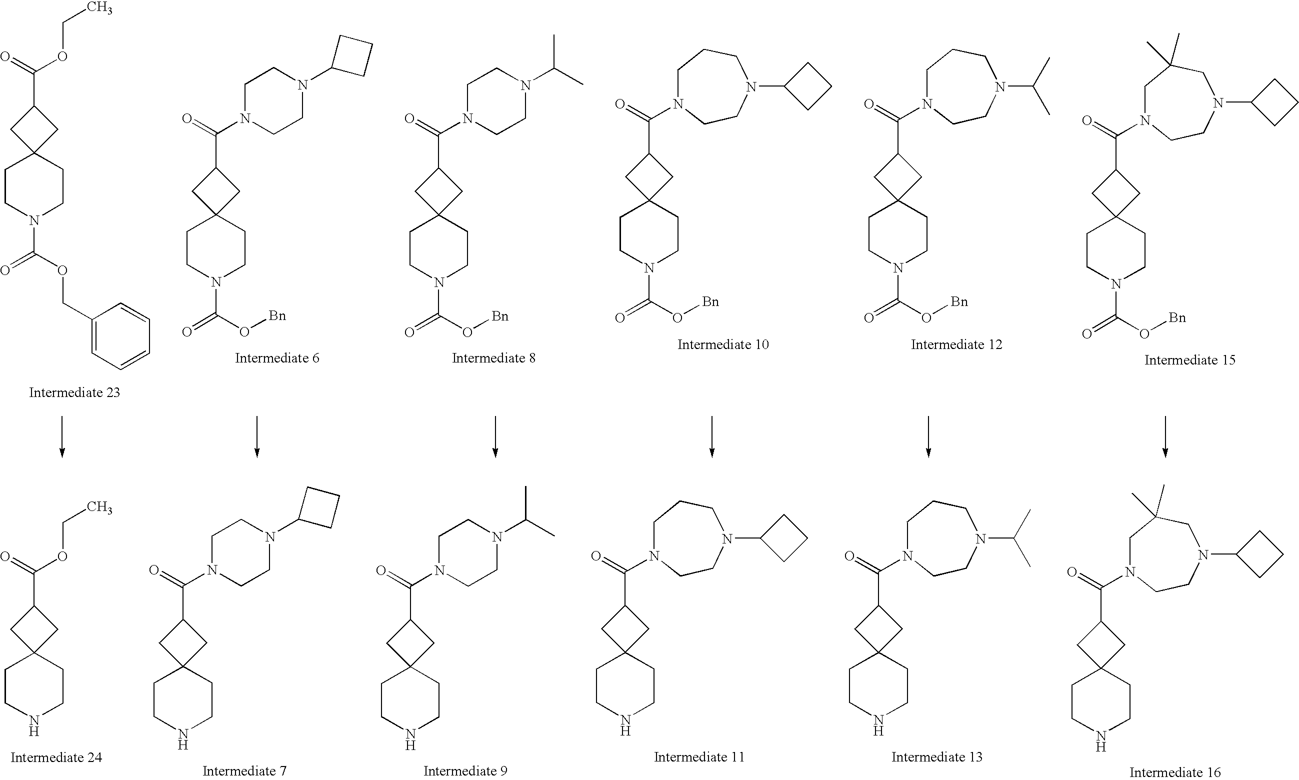

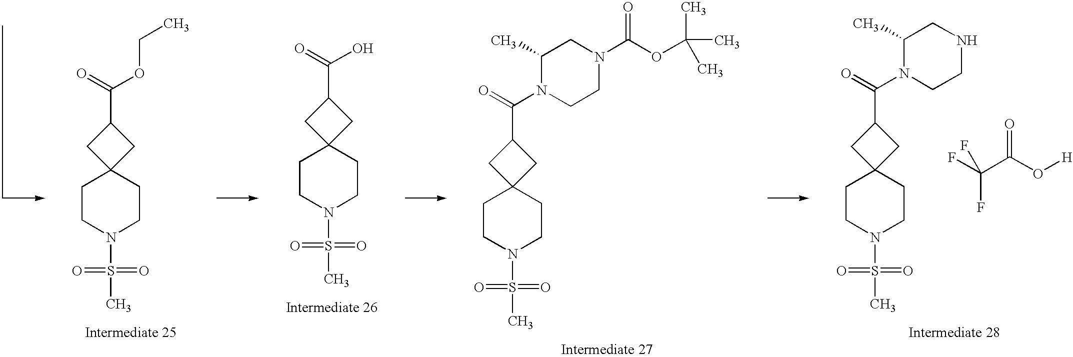

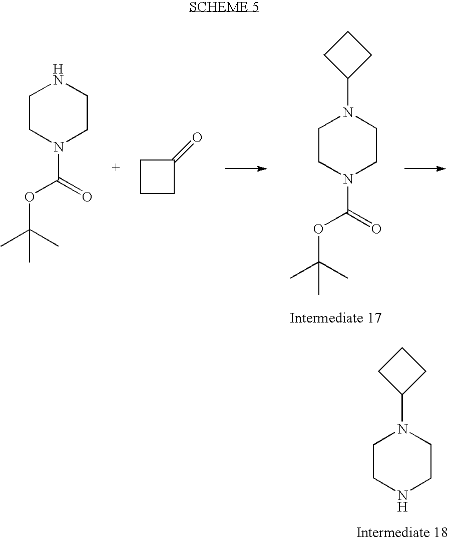

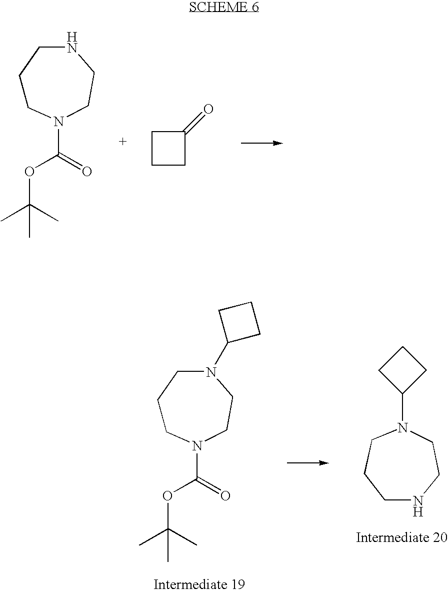

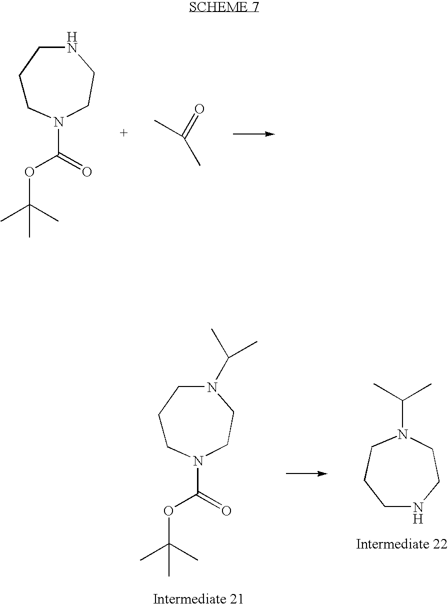

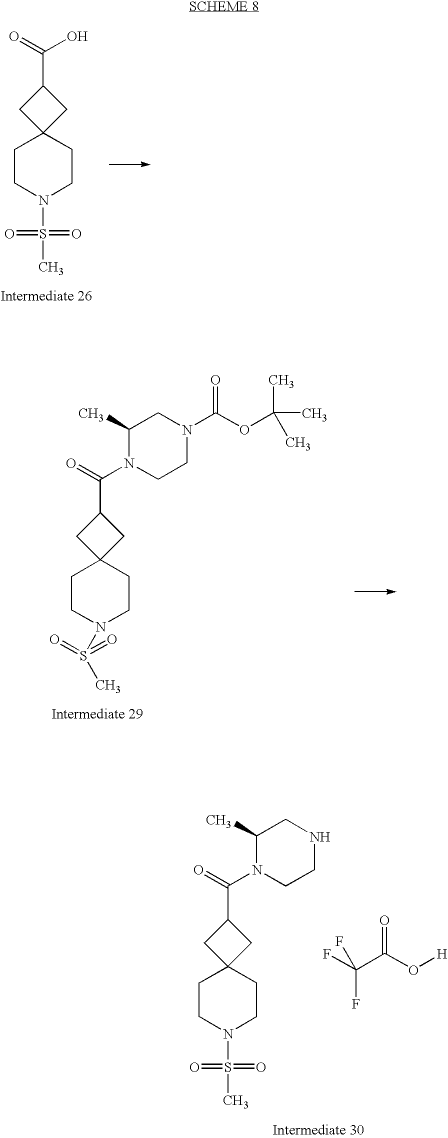

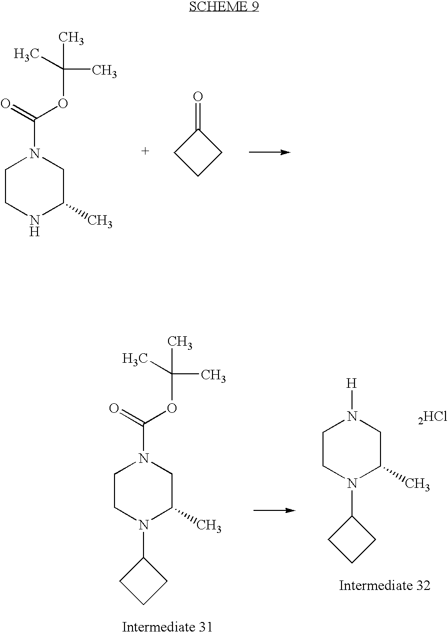

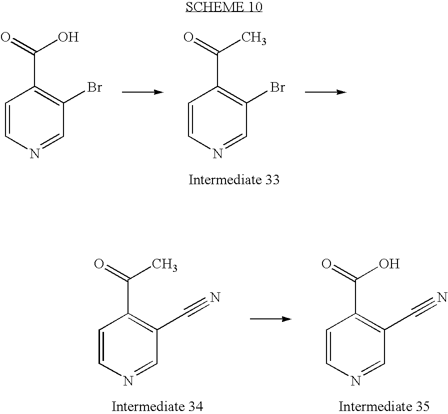

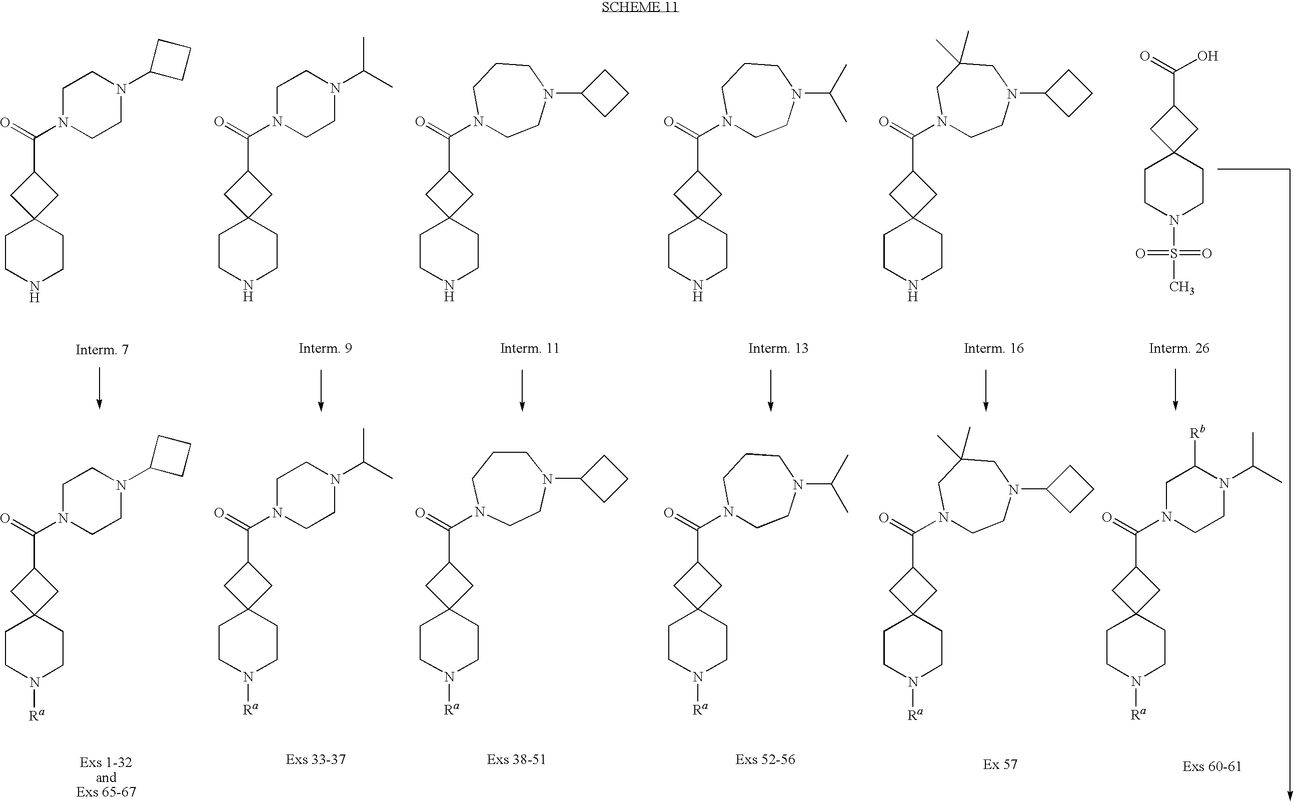

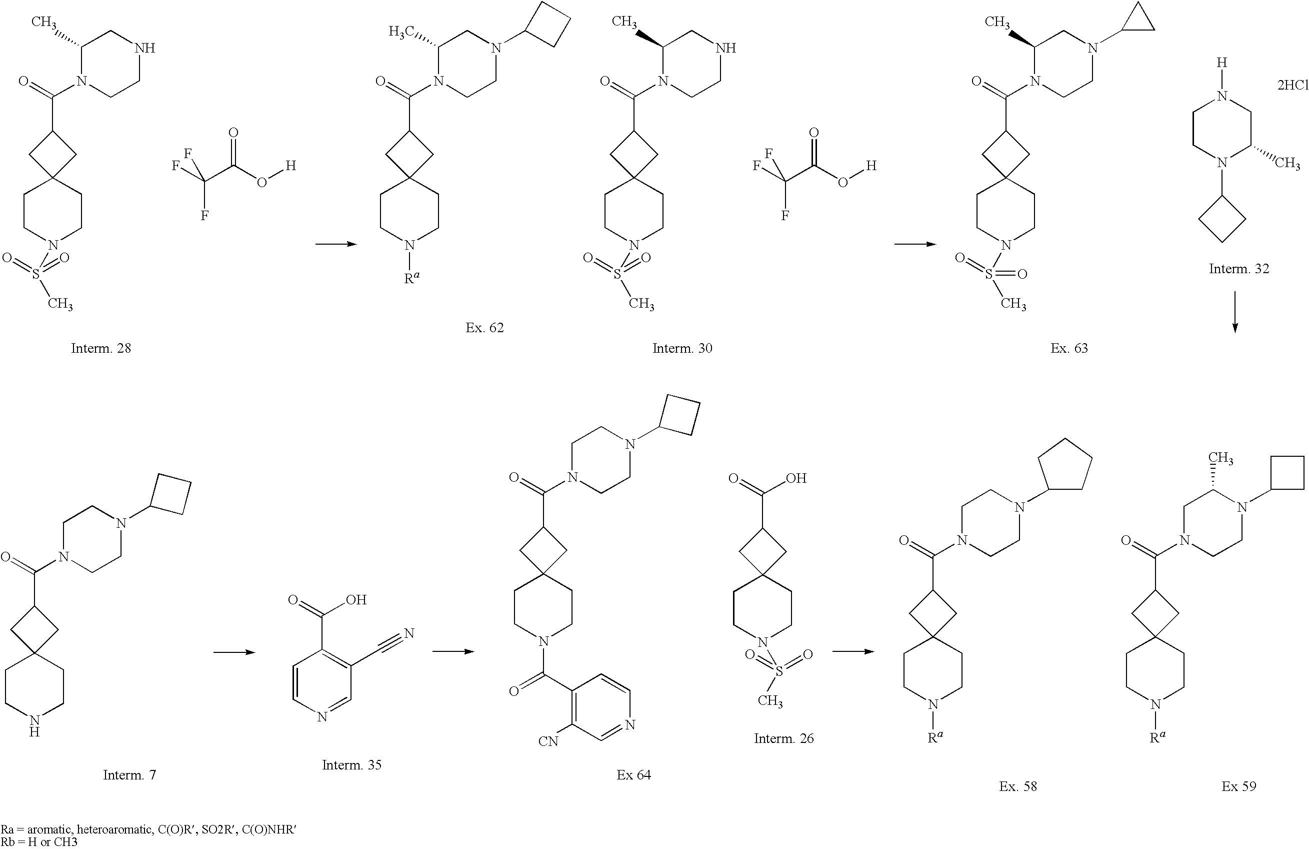

In another aspect, the disclosure is directed to a method for treating a disorder in which modulating the histamine H3 receptor is beneficial comprising administering to a warm-blooded animal in need of such treatment a therapeutically effective amount of at least one compound according to formula I, or pharmaceutically acceptable salts of formula I, or mixtures thereof:

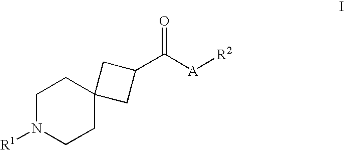

wherein

A is

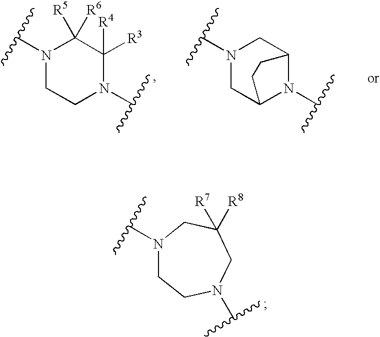

R¹ is aryl, 5- or 6-membered heteroaryl, —S(═O)₂R⁹, —C(═O)R¹⁰, or —C(═O)NR¹¹R¹²;

R² is C₃₋₆cycloalkyl or C₁₋₆alkyl;

R³, R⁴, R⁵, R⁶, R⁷, and R⁸ are each, independently, selected from hydrogen and —C₁-C₃alkyl;

R⁹ and R¹⁶ are each, independently, selected from C₁₋₆alkyl, 3-membered heterocycloalkyl, 4-membered heterocycloalkyl, 5-membered heterocycloalkyl, 6-membered heterocycloalkyl, 7-membered heterocycloalkyl, C₃₋₇cycloalkyl, aryl, 5-membered heteroaryl, 6-membered heteroaryl, (C₁₋₃alkyl)-(5-membered heteroaryl), and (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;

R¹¹ is cycloalkyl, aryl, 5-membered heteroaryl, 6-membered heteroaryl, (C₁₋₃alkyl)-(5-membered heteroaryl), or (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;

R¹² is H or C₁₋₆alkyl; and

R¹³ and R¹⁴ are each, independently, selected from H and C₁₋₃alkyl.

At least one compound in accordance with formula I, or pharmaceutically acceptable salts thereof, or mixtures thereof may be used to treat a wide range of conditions or disorders in which interacting with the histamine H3 receptor is beneficial. At least one formula I compound, or pharmaceutically acceptable salts thereof, or mixtures thereof may, for example, be useful to treat diseases of the central nervous system, the peripheral nervous system, the cardiovascular system, the pulmonary system, the gastrointestinal system, or the endocrinological system.

In one embodiment, at least one compound of formula I, or pharmaceutically acceptable salts thereof, or mixtures thereof modulates at least one histamine H3 receptor.

The terms “modulate”, “modulates”, “modulating”, or “modulation”, as used herein, refer to, for example, the activation (e.g., agonist activity) or inhibition (e.g., antagonist and inverse agonist activity) of at least one histamine H3 receptor.

In one embodiment, at least one compound of formula I, or pharmaceutically acceptable salts thereof, or mixtures thereof is an inverse agonist of at least one histamine H3 receptor.

In another embodiment, at least one compound of formula I, or pharmaceutically acceptable salts thereof, or mixtures thereof is an antagonist of at least one histamine H3 receptor.

Another embodiment provides a method for treating a disorder in which modulating the function of at least one histamine H3 receptor is beneficial comprising administering to a warm-blooded animal in need of such treatment a therapeutically effective amount of at least one compound according to formula I, or pharmaceutically acceptable salts thereof, or mixtures thereof.

In yet another embodiment, at least one compound in accordance with formula I, or pharmaceutically acceptable salts thereof, or mixtures thereof may be used as a medicament.

At least one compound in accordance with formula I, or pharmaceutically acceptable salts thereof, or mixtures thereof may be useful to treat at least one autoimmune disorder. Exemplary autoimmune disorders include, but are not limited to, for example, arthritis, skin grafts, organ transplants and similar surgical needs, collagen diseases, various allergies, tumors and viruses.

At least one compound in accordance with formula I, or pharmaceutically acceptable salts thereof, or mixtures thereof may be useful to treat at least one psychiatric disorder.

Exemplary psychiatric disorders include, but are not limited to, for example, Psychotic Disorder(s) and Schizophrenia Disorder(s), such as, for example, Schizoaffective Disorder(s), Delusional Disorder(s), Brief Psychotic Disorder(s), Shared Psychotic Disorder(s), and Psychotic Disorder(s) Due to a General Medical Condition; Dementia and other Cognitive Disorder(s); Anxiety Disorder(s), such as, for example, Panic Disorder(s) Without Agoraphobia, Panic Disorder(s) With Agoraphobia, Agoraphobia Without History of Panic Disorder(s), Specific Phobia, Social Phobia, Obsessive-Compulsive Disorder(s), Stress related Disorder(s), Posttraumatic Stress Disorder(s), Acute Stress Disorder(s), Generalized Anxiety Disorder(s) and Generalized Anxiety Disorder(s) Due to a General Medical Condition; Mood Disorder(s), such as, for example, a) Depressive Disorder(s) (including but not limited to, for example, Major Depressive Disorder(s) and Dysthymic Disorder(s)), b) Bipolar Depression and/or Bipolar mania, such as, for example, Bipolar I (which includes, but is not limited to those with manic, depressive or mixed episodes), and Bipolar II, c) Cyclothymiac's Disorder(s), and d) Mood Disorder(s) Due to a General Medical Condition; Sleep Disorder(s), such as, for example, narcolepsy; Disorder(s) Usually First Diagnosed in Infancy, Childhood, or Adolescence including, but not limited to, for example, Mental Retardation, Downs Syndrome, Learning Disorder(s), Motor Skills Disorder(s), Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s); Substance-Related Disorder(s) including, but not limited to, for example, Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s)s, Opiod-Related Disorder(s)s, Phencyclidine (or Phencyclidine-Like)-Related Disorder(s), and Sedative-, Hypnotic- or Anxiolytic-Related Disorder(s); Attention-Deficit and Disruptive Behavior Disorder(s); Eating Disorder(s), such as, for example, obesity; Personality Disorder(s) including, but not limited to, for example, Obsessive-Compulsive Personality Disorder(s); Impulse-Control Disorder(s); Tic Disorders including, but not limited to, for example Tourette's Disorder, Chronic motor or vocal tic disorder; and Transient Tic Disorder.

At least one of the above psychiatric disorders is defined, for example, in the American Psychiatric Association: Diagnostic and Statistical Manual of Mental Disorders, Fourth Edition, Text Revision, Washington, D.C., American Psychiatric Association, 2000.

At least one compound in accordance with formula I, or pharmaceutically acceptable salts thereof, or mixtures thereof may be useful i) to treat obesity or being overweight (e.g., promotion of weight loss and maintenance of weight loss), eating disorders (e.g., binge eating, anorexia, bulimia and compulsive), and/or cravings (for drugs, tobacco, alcohol, any appetizing macronutrients or non-essential food items); ii) to prevent weight gain (e.g., medication-induced or subsequent to cessation of smoking); and/or iii) to modulate appetite and/or satiety.

At least one compound in accordance with formula I, or pharmaceutically acceptable salts thereof, or mixtures thereof may be suitable for treating obesity by reducing appetite and body weight and/or maintaining weight reduction and preventing rebound.

At least one compound in accordance with formula I, or pharmaceutically acceptable salts thereof, or mixtures thereof may be used to prevent or reverse medication-induced weight gain, e.g. weight gain caused by antipsychotic (neuroleptic) treatment(s); and/or weight gain associated with smoking cessation.

At least one compound in accordance with formula I, or pharmaceutically acceptable salts thereof, or mixtures thereof may be useful to treat at least one Neurodegenerative Disorder.

Exemplary Neurodegenerative Disorders include, but are not limited to, for example, Alzheimer's Disease (AD); Dementia, which includes, but is not limited to, for example, Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases; Cognitive Deficit in Schizophrenia (CDS); Mild Cognitive Impairment (MCI); Age-Associated Memory Impairment (AAMI); Age-Related Cognitive Decline (ARCD); Cognitive Impairment No Dementia (CIND); Multiple Sclerosis; Parkinson's Disease (PD); postencephalitic parkinsonism; Huntington's Disease; amyotrophic lateral sclerosis (ALS); motor neuron diseases (MND); Multiple System Atrophy (MSA); Corticobasal Degeneration; Progressive Supranuclear Paresis; Guillain-Barr Syndrome (GBS); and Chronic Inflammatory Demyelinating Polyneuropathy (CIDP).

At least one compound in accordance with formula I, or pharmaceutically acceptable salts thereof, or mixtures thereof may be useful to treat at least one Neuroinflammatory Disorder.

Exemplary Neuroinflammatory Disorders include, but are not limited to, for example, Multiple Sclerosis (MS), which includes, but is not limited to, for example, Relapse Remitting Multiple Sclerosis (RRMS), Secondary Progressive Multiple Sclerosis (SPMS), and Primary Progressive Multiple Sclerosis (PPMS); Parkinson's disease; Multiple System Atrophy (MSA); Corticobasal Degeneration; Progressive Supranuclear Paresis; Guillain-Barré Syndrome (GBS); and chronic inflammatory demyelinating polyneuropathy (CIDP).

At least one compound in accordance with formula I, or pharmaceutically acceptable salts thereof, or mixtures thereof may be useful to treat at least one Attention-Deficit and Disruptive Behavior Disorder.

Exemplary Attention-Deficit and Disruptive Behavior Disorders include, but are not limited to, for example, attention deficit disorder (ADD), attention deficit hyperactivity disorder (ADHD), and affective disorders.

In one embodiment, at least one compound in accordance with formula I, or pharmaceutically acceptable salts thereof, or mixtures thereof may be used in the manufacture of a medicament for the treatment of at least one psychiatric, neurodegenerative, neuroinflammatory, or Attention-Deficit and Disruptive Behaviour Disorder described hereinabove.

At least one compound in accordance with formula I, or pharmaceutically acceptable salts thereof, or mixtures thereof may be useful to treat pain; acute and chronic pain disorders including but not limited to, for example, Widespread pain, Localized pain, Nociceptive pain, Inflammatory pain, Central pain, Central and peripheral neuropathic pain, Central and peripheral neurogenic pain, Central and peripheral neuralgia, Low back pain, Postoperative pain, Visceral pain, and Pelvic pain; Allodynia; Anesthesia dolorosa; Causalgia; Dysesthesia; Fibromyalgia; Hyperalgesia; Hyperesthesia; Hyperpathia; Ischemic pain; Sciatic pain; Pain associated with cystitis including, but not limited to, interstitial cystitis; Pain associated with multiple sclerosis; Pain associated with arthritis; Pain associated with osteoarthritis; Pain associated with rheumatoid arthritis; and Pain associated with cancer.

In one embodiment, at least one compound in accordance with formula I, or pharmaceutically acceptable salts thereof, or mixtures thereof may be used for the manufacture of a medicament for the treatment of at least one autoimmune disorder, psychiatric disorder, obesity disorder, eating disorder, craving disorder, neurodegenerative disorder, neuroinflammatory disorder, Attention-Deficit and Disruptive Behaviour Disorder, and/or pain disorder described hereinabove.

In another embodiment, at least one compound in accordance with formula I, or pharmaceutically acceptable salts thereof, or mixtures thereof may be used for the manufacture of a medicament for the treatment of at least one disorder selected from cognitive deficit in schizophrenia, narcolepsy, attention deficit hyperactivity disorder, obesity, pain, and Alzheimer's disease.

A further embodiment provides a compound according to formula I, or pharmaceutically acceptable salts thereof, or mixtures thereof for the treatment of at least one disorder selected from cognitive deficit in schizophrenia, narcolepsy, obesity, attention deficit hyperactivity disorder, pain, and Alzheimer's disease.

A still yet further embodiment provides a compound according to formula I or pharmaceutically acceptable salts thereof, or mixtures thereof for the treatment of at least one disorder selected from cognitive deficit in schizophrenia and Alzheimer's disease.

Another embodiment provides a method for treating at least one autoimmune disorder, psychiatric disorder, obesity disorder, eating disorder, craving disorder, neurodegenerative disorder, neuroinflammatory disorder, Attention-Deficit and Disruptive Behaviour Disorder, and/or pain disorder in a warm-blooded animal, comprising administering to said animal in need of such treatment a therapeutically effective amount of at least one compound according to formula I or pharmaceutically acceptable salts thereof, or mixtures thereof.

Another embodiment provides a method for treating at least one disorder selected from cognitive deficit in schizophrenia, narcolepsy, obesity, attention deficit hyperactivity disorder, pain, and Alzheimer's disease in a warm-blooded animal, comprising administering to said animal in need of such treatment a therapeutically effective amount of at least one compound according to formula I or pharmaceutically acceptable salts thereof, or mixtures thereof.

Another embodiment provides a method for treating cognitive deficit in schizophrenia in a warm-blooded animal comprising administering to said animal in need of such treatment a therapeutically effective amount of at least one compound according to formula I, or pharmaceutically acceptable salts thereof, or mixtures thereof.

Another embodiment provides a method for treating obesity in a warm-blooded animal comprising administering to said animal in need of such treatment a therapeutically effective amount of at least one compound according to formula I, or pharmaceutically acceptable salts thereof, or mixtures thereof.

Another embodiment provides a method for treating narcolepsy in a warm-blooded animal comprising administering to said animal in need of such treatment a therapeutically effective amount of at least one compound according to formula I, or pharmaceutically acceptable salts thereof, or mixtures thereof.

Another embodiment provides a method for treating Alzheimer's disease in a warm-blooded animal, comprising administering to said animal in need of such treatment a therapeutically effective amount of at least one compound according to formula I, or pharmaceutically acceptable salts thereof, or mixtures thereof.

Another embodiment provides a method for treating attention deficit hyperactivity disorder in a warm-blooded animal comprising administering to said animal in need of such treatment a therapeutically effective amount of at least one compound according to formula I, or pharmaceutically acceptable salts thereof, or mixtures thereof.

Another embodiment provides a method for treating a pain disorder in a warm-blooded animal, comprising administering to said animal in need of such treatment a therapeutically effective amount of at least one compound according to formula I, or pharmaceutically acceptable salts thereof, or mixtures thereof.

In one embodiment, the warm-blooded animal is a mammalian species including, but not limited to, for example, humans and domestic animals, such as, for example, dogs, cats, and horses.

In a further embodiment, the warm-blooded animal is a human.

Another embodiment provides the use of at least one compound in accordance with formula I, or pharmaceutically acceptable salts thereof, or mixtures thereof in therapy.

Yet an even further embodiment provides the use of at least one compound in accordance with formula I, or pharmaceutically acceptable salts thereof, or mixtures thereof in the manufacture of a medicament for use in therapy.

As used herein, the term “therapy” also includes “prophylaxis” unless specifically indicated to the contrary.

In yet another embodiment at least one compound in accordance with formula I, or pharmaceutically acceptable salts thereof, or mixtures thereof, or a pharmaceutical composition or formulation comprising at least one compound in accordance with formula I, or pharmaceutically acceptable salts thereof, or mixtures thereof may be administered concurrently, simultaneously, sequentially or separately with at least one other pharmaceutically active compound selected from the following:

(i) antidepressants, such as, for example, agomelatine, amitriptyline, amoxapine, bupropion, citalopram, clomipramine, desipramine, doxepin, duloxetine, escitalopram, fluvoxamine, fluoxetine, gepirone, imipramine, ipsapirone, isocarboxazid, maprotiline, mirtazepine, nortriptyline, nefazodone, paroxetine, phenelzine, protriptyline, ramelteon, reboxetine, robalzotan, selegiline, sertraline, sibutramine, thionisoxetine, tranylcypromaine, trazodone, trimipramine, venlafaxine, and equivalents and pharmaceutically active isomer(s) and metabolite(s) thereof;

(ii) antipsychotics, such as, for example, amisulpride, aripiprazole, asenapine, benzisoxidil, bifeprunox, carbamazepine, clozapine, chlorpromazine, debenzapines, dibenzapine, divalproex, droperidol, fluphenazine, haloperidol, iloperidone, loxapine, mesoridazine, molindone, olanzapine, paliperidone, perphenazine, phenothiazine, phenylbutylpiperidine, pimozide, prochlorperazine, quetiapine, risperidone, sertindole, sulpiride, suproclone, thioridazine, thiothixene, trifluoperazine, trimetozine, valproate, valproic acid, zotepine, ziprasidone, and equivalents and pharmaceutically active isomer(s) and metabolite(s) thereof;

(iii) anxiolytics, such as, for example, alnespirone, azapirones, benzodiazepines, and barbiturates, such as, for example, adinazolam, alprazolam, balezepam, bentazepam, bromazepam, brotizolam, buspirone, clonazepam, clorazepate, chlordiazepoxide, cyprazepam, diazepam, estazolam, fenobam, flunitrazepam, flurazepam, fosazepam, lorazepam, lormetazepam, meprobamate, midazolam, nitrazepam, oxazepam, prazepam, quazepam, reclazepam, suriclone, tracazolate, trepipam, temazepam, triazolam, uldazepam, zolazepam, and equivalents and pharmaceutically active isomer(s) and metabolite(s) thereof;

(iv) anticonvulsants, such as, for example, carbamazepine, oxcarbazepine, valproate, lamotrigine, gabapentin, topiramate, phenyloin, ethosuximide, and equivalents and pharmaceutically active isomer(s) and metabolite(s) thereof;

(v) Alzheimer's therapies, such as, for example, donepezil, galantamine, memantine, rivastigmine, tacrine, and equivalents and pharmaceutically active isomer(s) and metabolite(s) thereof;

(vi) Parkinson's therapies and agents for the treatment of extrapyramidal symptoms, such as, for example, levodopa, carbidopa, amantadine, pramipexole, ropinirole, pergolide, cabergoline, apomorphine, bromocriptine, MAOB inhibitors (i.e. selegine and rasagiline), COMT inhibitors (i.e. entacapone and tolcapone), alpha-2 inhibitors, anticholinergics (i.e., benztropine, biperiden, orphenadrine, procyclidine, and trihexyphenidyl), dopamine reuptake inhibitors, NMDA antagonists, Nicotine agonists, Dopamine agonists, and inhibitors of neuronal nitric oxide synthase, and equivalents and pharmaceutically active isomer(s) and metabolite(s) thereof;

(vii) migraine therapies, such as, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(viii) stroke therapies, such as, for example, abciximab, activase, NXY-059, citicoline, crobenetine, desmoteplase, repinotan, traxoprodil, and equivalents and pharmaceutically active isomer(s) and metabolite(s) thereof;

(ix) urinary incontinence therapies, such as, for example, darafenacin, dicyclomine, falvoxate, imipramine, desipramine, oxybutynin, propiverine, propanthedine, robalzotan, solifenacin, alfazosin, doxazosin, terazosin, tolterodine, and equivalents and pharmaceutically active isomer(s) and metabolite(s) thereof;

(x) neuropathic pain therapies, such as, for example, gabapentin, lidoderm, pregablin, and equivalents and pharmaceutically active isomer(s) and metabolite(s) thereof;

(xi) nociceptive pain therapies, such as, for example, celecoxib, codeine, diclofenac, etoricoxib, fentanyl, hydrocodone, hydromorphone, levo-alpha-acetylmethadol, loxoprofen, lumiracoxib, meperidine, methadone, morphine, naproxen, oxycodone, paracetamol, propoxyphene, rofecoxib, sufentanyl, valdecoxib, and equivalents and pharmaceutically active isomer(s) and metabolite(s) thereof;

(xii) insomnia therapies and sedative hypnotics, such as, for example, agomelatine, allobarbital, alonimid, amobarbital, benzoctamine, butabarbital, capuride, chloral hydrate, clonazepam, chlorazepate, cloperidone, clorethate, dexclamol, estazolam, eszopiclone, ethchlorvynol, etomidate, flurazepam, glutethimide, halazepam, hydroxyzine, mecloqualone, melatonin, mephobarbital, methaqualone, midaflur, midazolam, nisobamate, pagoclone, pentobarbital, perlapine, phenobarbital, propofol, quazepam, ramelteon, roletamide, suproclone, temazepam, triazolam, triclofos, secobarbital, zaleplon, zolpidem, zopiclone and equivalents and pharmaceutically active isomer(s) and metabolite(s) thereof;

(xiii) mood stabilizers, such as, for example, carbamazepine, divalproex, gabapentin, lamotrigine, lithium, olanzapine, oxycarbazepine, quetiapine, valproate, valproic acid, verapamil, and equivalents and pharmaceutically active isomer(s) and metabolite(s) thereof;

(xiv) obesity therapies, such as, for example, anti-obesity drugs that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, and G-I motility; very low calorie diets (VLCD); and low-calorie diets (LCD);

(xv) therapeutic agents useful in treating obesity associated disorders, such as, for example, biguanide drugs, insulin (synthetic insulin analogues) and oral antihyperglycemics (these are divided into prandial glucose regulators and alpha-glucosidase inhibitors), PPAR modulating agents, such as, for example, PPAR alpha and/or gamma agonists; sulfonylureas; cholesterol-lowering agents, such as, for example, inhibitors of HMG-CoA reductase (3-hydroxy-3-methylglutaryl coenzyme A reductase); an inhibitor of the ileal bile acid transport system (IBAT inhibitor); a bile acid binding resin; bile acid sequestering agent, such as, for example, colestipol, cholestyramine, or cholestagel; a CETP (cholesteryl ester transfer protein) inhibitor; a cholesterol absorption antagonist; a MTP (microsomal transfer protein) inhibitor; a nicotinic acid derivative, including slow release and combination products; a phytosterol compound; probucol; an anti-coagulant; an omega-3 fatty acid; an anti-obesity therapy, such as, for example, sibutramine, phentermine, orlistat, bupropion, ephedrine, and thyroxine; an antihypertensive, such as, for example, an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic, and a vasodilator; a melanin concentrating hormone (MCH) modulator; an NPY receptor modulator; an orexin receptor modulator; a phosphoinositide-dependent protein kinase (PDK) modulator; modulators of nuclear receptors, such as, for example, LXR, FXR, RXR, GR, ERRα, β, PPARα, β, γ and RORalpha; a monoamine transmission-modulating agent, such as, for example, a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA); a serotonin receptor modulator; a leptin/leptin receptor modulator; a ghrelin/ghrelin receptor modulator; a DPP-IV inhibitor; and equivalents and pharmaceutically active isomer(s), metabolite(s), and pharmaceutically acceptable salts, solvates, and prodrugs thereof.

(xvi) agents for treating ADHD, such as, for example, amphetamine, methamphetamine, dextroamphetamine, atomoxetine, methylphenidate, dexmethylphenidate, modafinil, and equivalents and pharmaceutically active isomer(s) and metabolite(s) thereof; and

(xvii) agents used to treat substance abuse disorders, dependence, and withdrawal, such as, for example, nicotine replacement therapies (i.e., gum, patches, and nasal spray); nicotinergic receptor agonists, partial agonists, and antagonists, (e.g. varenicline); acomprosate, bupropion, clonidine, disulfuram, methadone, naloxone, naltrexone, and equivalents and pharmaceutically active isomer(s) and metabolite(s) thereof.

The above other pharmaceutically active compound, when employed in combination with compounds of formula I, or pharmaceutically acceptable salts thereof, or mixtures thereof may be used, for example, in the amounts indicated in the Physicians' Desk Reference (PDR) or as otherwise determined by one of ordinary skill in the art.

Compound(s) in accordance with formula I, or pharmaceutically acceptable salts thereof, or mixtures thereof may be administered by any means suitable for the condition to be treated, which can depend on the quantity of formula I, or pharmaceutically acceptable salts thereof, or mixtures thereof to be delivered.

Compound(s) in accordance with formula I, or pharmaceutically acceptable salts thereof, or mixtures thereof may be administered in the form of a conventional pharmaceutical composition by any route including, but not limited to, for example, orally, intramuscularly, subcutaneously, topically, intranasally, epidurally, intraperitoneally, intrathoracially, intravenously, intrathecally, intracerebroventricularly, and injecting into the joints.

In one embodiment, the route of administration is orally, intravenously or intramuscularly.

An “effective amount” of formula I, or pharmaceutically acceptable salts thereof, or mixtures thereof may be determined by one of ordinary skill in the art, and includes exemplary dosage amounts for a mammal of from about 0.01 to about 20 mg/kg/day, preferably less than about 10 mg/kg/day, in a single dose or in or in the form of individual divided doses. Exemplary dosage amounts for an adult human are from about 0.1 to 5 (for example, 1) mg/kg of body weight of active compound per day, which can be administered in a single dose or in the form of individual divided doses, such as from 1 to 4 times per day.

The specific dose level and frequency of dosage for any particular subject, however, may vary and generally depends on a variety of factors, including, but not limited to, for example, the bioavailability of the specific formula I compound(s), or pharmaceutically acceptable salts thereof, or mixtures thereof in the administered form; metabolic stability and length of action of the specific formula I compound(s), or pharmaceutically acceptable salts thereof, or mixtures thereof; species, age, body weight, general health, sex, and diet of the subject; mode and time of administration; rate of excretion; drug combination; and severity of the particular condition.

One embodiment provides a pharmaceutical composition comprising at least one compound in accordance with formula I, or pharmaceutically acceptable salts thereof, or mixtures thereof and at least one pharmaceutically-acceptable carrier and/or diluent.

Another embodiment provides a method for treating at least one disorder selected from cognitive deficient in schizophrenia, narcolepsy, obesity, attention deficit hyperactivity disorder, and Alzheimer's disease in a warm-blooded animal, comprising administering to said animal in need of such treatment a pharmaceutical composition comprising a therapeutically effective amount of a compound according to formula I, or pharmaceutically acceptable salts thereof, or mixtures thereof and at least one pharmaceutically-acceptable carrier and/or diluent.

Acceptable solid pharmaceutical compositions include, but are not limited to, for example, powders, tablets, dispersible granules, capsules, cachets, and suppositories.

In a solid pharmaceutical composition, pharmaceutically acceptable carriers include, but are not limited to, for example, at least one solid, at least one liquid, and mixtures thereof. The solid carrier can also be a diluent, flavoring agent, solubilizer, lubricant, suspending agent, binder, encapsulating material, and/or table disintegrating agent. Suitable carriers, include, but are not limited to, for example, magnesium carbonate; magnesium stearate; talc; lactose; sugar; pectin; dextrin; starch; tragacanth; methyl cellulose; sodium carboxymethyl cellulose; a low-melting wax; cocoa butter; and mixtures thereof.

A powder can be prepared by, for example, mixing a finely divided solid with at least one finely divided compound of formula I, or pharmaceutically acceptable salts thereof, or mixtures thereof.

A tablet can be prepared by, for example, mixing at least one formula I compound, or pharmaceutically acceptable salts thereof, or mixtures thereof in suitable proportions with a pharmaceutically acceptable carrier having the necessary binding properties and compacted into the desired shape and size.

A suppository can be prepared by, for example, mixing at least one compound of formula I, or pharmaceutically acceptable salts thereof, or mixtures thereof with at least one suitable non-irritating excipient that is liquid at rectal temperature but solid at a temperature below rectal temperature, wherein the non-irritating excipient is first melted and the formula I compound, or pharmaceutically acceptable salts thereof, or mixtures thereof dispersed therein. The molten homogeneous mixture in then poured into convenient sized molds and allowed to cool and solidify. Exemplary non-irritating excipients include, but are not limited to, for example, cocoa butter; glycerinated gelatin; hydrogenated vegetable oils; mixtures of polyethylene glycols of various molecular weights; and fatty acid esters of polyethylene glycol.

Acceptable liquid pharmaceutical compositions include, but are not limited to, for example, solutions, suspensions, and emulsions. For example, sterile water or water propylene glycol solutions of at least one compound in accordance with formula I, or pharmaceutically acceptable salts thereof, or mixtures thereof are liquid pharmaceutical compositions suitable for parenteral administration. Liquid compositions can also be formulated in solution in aqueous polyethylene glycol solution.

Aqueous solutions for oral administration can be prepared by dissolving at least one compound in accordance with formula I, or pharmaceutically acceptable salts thereof, or mixtures thereof in water and adding suitable colorants, flavoring agents, stabilizers, and/or thickening agents as desired.

Aqueous suspensions for oral administration can be prepared by dispersing at least one finely divided compound of formula I, or pharmaceutically acceptable salts thereof, or mixtures thereof in water together with a viscous material, such as, for example, a natural synthetic gum, resin, methyl cellulose, and sodium carboxymethyl cellulose.

In one embodiment, the pharmaceutical composition contains from about 0.05% to about 99% w (percent by weight) of at least one compound in accordance with formula I, or pharmaceutically acceptable salts thereof, or mixtures thereof. All percentages by weight being based on total composition.

In another embodiment, the pharmaceutical composition contains from about 0.10% to about 50% w(percent by weight) of at least one compound in accordance with formula I, or pharmaceutically acceptable salts thereof, or mixtures thereof. All percentages by weight being based on total composition.

Another embodiment, provides a pharmaceutical composition comprising a compound of formula I, or pharmaceutically acceptable salts thereof, or mixtures thereof and a pharmaceutically acceptable carrier and/or diluent for therapy.

Further, there is provided a pharmaceutical composition comprising a compound of formula I, or pharmaceutically acceptable salts thereof, or mixtures thereof in association with a pharmaceutically acceptable carrier and/or diluent for use in any of the conditions discussed above.

In a further aspect, the present invention provides a method of preparing a compound of formula I, or pharmaceutically acceptable salts thereof, or mixtures thereof.

### Biological Evaluation

At least one compound of formula I including the compounds described in the Examples hereof, when tested in at least one in vitro assay as substantially described below is active towards H3 receptors. Particularly, at least one compound of the invention is an effective H3 receptor ligand. The in vitro activity may be related to in vivo activity but may not be linearly correlated with binding affinity. In the in vitro assay, a compound can be tested for its activity toward H3 receptors and IC₅₀ obtained to determine the activity for a particular compound toward the H3 receptor.

### Guanosine 5′-O-(3-[³⁵S]thio)triphosphate [GTPγS] Binding Assay

A GTPγS binding assay can be used to investigate antagonist properties of compounds in CHO cells (Chinese Hamster Ovary) transfected with human Histamine H3 receptor (hH3R). Membranes from CHO cells expressing hH3R (10 μg/well) are diluted in GTPγS assay buffer (20 mM Hepes, 10 mM MgCl₂, 100 mM NaCl, pH 7.4) and preincubated with saponine (3 μg/ml), GDP (10 μM) and PVT-WGA SPA beads (125 μg/well) (Amersham) for 30 minutes. To determine antagonist activity, (R)-α-methyl histamine (30 nM) is added in 96 well SPA plate with [³⁵S]GTPγS (0.2 nM) and various concentration of H3R antagonists. The GTPγS binding assay is started with addition of the mixture membrane/saponine/GDP and incubated for 90 minutes at room temperature. The amount of bound [³⁵S]GTPγS is determined by using the MicroBeta Trilux counter (PerkinElmer). The percentage of [³⁵S]GTPγS bound in each sample is calculated as a percentage of that bound control sample incubated in absence of H3 antagonist. Duplicate determinations are obtained for each concentration, and the data are analyzed using ExcelFit4 to obtain the IC₅₀.

### IC₅₀ Values

At least one formula I compound in accordance with the present invention may have an IC₅₀ value of less than about 1 μM. In a further embodiment, at least one compound of formula I may have activity in at least one of the above referenced assays via an IC₅₀ value of between about 1 nm to about 1 μM. In an even further embodiment, at least one compound of formula I may have activity in at least one of the above referenced assays via an IC₅₀ value of between about 2 nM to about 100 nM. In yet a further embodiment, at least one compound of formula I may have activity in at least one of the above referenced assays via an IC₅₀ value of between about 2 nM and 50 nM. In one embodiment, at least one compound of formula I may have activity in at least one of the above referenced assays via an IC₅₀ value of less than about 100 nM. In another embodiment, at least one compound of formula I may have activity in at least one of the above referenced assays via an IC₅₀ value of less than about 50 nM. In yet another embodiment, at least one compound of formula I may have activity in at least one of the above referenced assays via an IC₅₀ value of less than about 10 nM.

Set forth in Table 1 hereinbelow for the Example 1-67 compounds are IC₅₀ values that were generated in accordance with the GTPγS Binding Assay as essentially described hereinabove.

TABLE 1

| | GTPγS Binding |
|---|---|
| EX No. | IC₅₀ (nM) |
| 1 | 17.77 |
| 2 | 27.05 |
| 3 | 72.87 |
| 4 | 15.38 |
| 5 | 7.29 |
| 6 | 6.56 |
| 7 | 52.91 |
| 8 | 22.89 |
| 9 | 20.76 |
| 10 | 29.56 |
| 11 | 16.89 |
| 12 | 26.23 |
| 13 | 28.13 |
| 14 | 41.50 |
| 15 | 31.33 |
| 16 | 80.41 |
| 17 | 34.95 |
| 18 | 53.96 |
| 19 | 24.54 |
| 20 | 48.04 |
| 21 | 18.24 |
| 22 | 12.62 |
| 23 | 6.06 |
| 24 | 21.76 |
| 25 | 12.51 |
| 26 | 23.81 |
| 27 | 12.60 |
| 28 | 13.76 |
| 29 | 19.74 |
| 30 | 48.36 |
| 31 | 24.53 |
| 32 | 47.70 |
| 33 | 33.85 |
| 34 | 66.38 |
| 35 | 61.94 |
| 36 | 67.42 |
| 37 | 114.40 |
| 38 | 8.90 |
| 39 | 9.97 |
| 40 | 6.22 |
| 41 | 8.71 |
| 42 | 14.99 |
| 43 | 8.87 |
| 44 | 8.45 |
| 45 | 7.61 |
| 46 | 5.75 |
| 47 | 8.27 |
| 48 | 12.68 |
| 49 | 5.28 |
| 50 | 7.62 |
| 51 | 5.02 |
| 52 | 56.4 |
| 53 | 160.3 |
| 54 | 161.2 |
| 55 | 115 |
| 56 | 268.6 |
| 57 | 706.7 |
| 58 | 62.97 |
| 59 | 368.5 |
| 60 | 177.4 |
| 61 | 178.9 |
| 62 | 4.249 |
| 63 | 21.23 |
| 64 | 16.77 |
| 65 | 51.68 |
| 66 | 10.08 |
| 67 | 22.86 |

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather defined by the claims appended hereto.

All temperatures are in degrees Celsius (° C.) and are uncorrected.

Unless otherwise noted, commercial reagents used in preparing the example compounds were used as received without additional purification.

Unless otherwise noted, the solvents used in preparing the example compounds were commercial anhydrous grades and were used without further drying or purification.

All starting materials are commercially available, unless stated otherwise.

The name of the products were determined using the naming software included in CambridgeSoft E-Notebook version 9.2 (Chemoffice 9.0.7).

The following abbreviations are employed herein:

BINAP: 2,2′ bis(diphenylphosphino)-1,1′-binaphthyl; br.: broad; Bu: butyl; Celite®: brand of diatomaceous earth filtering agent, registered trader of Celite Corporation; calcd: calculated; d: doublet; dd: doublet of doublet; ddd: doublet of doublet of doublet; DCE: dichloroethane; DCM: dichloromethane; DIEA: N-ethyl-N-isopropylpropan-2-amine; DMAc: dimethylacetamide; DME: dimethyl ether; DMF: N,N-dimethyl formamide; dq: doublet of quartet; dt: doublet of triplet; EDC: 1-ethyl-3-(3-dimethylaminopropyl) carbodiimide hydrochloride; ESI: electrospray ion source; Et₃N: triethylamine; EtOAc: ethyl acetate; EtOH: Ethanol; g: gram; h: hour(s); ¹H NMR: proton nuclear magnetic resonance; HBTU: O-Benzotriazole-N,N,N′,N′-tetramethyl-uronium-hexafluoro-phosphate; HOBT: N-Hydroxybenzotriazole; HPLC: high pressure liquid chromatography; HRMS: high resolution mass spectrometry; L: liter; m: multiplet; M: molar; mL: milliliter; MeOH: methanol; mg: milligram; MHz: megahertz; min minute(s); mmol millimole; mol: mole; MPLC: medium pressure liquid chromatography; MS: mass spectrometry; MTBE: methyl tert-butyl ether; NaOH: sodium hydroxide; Pd/C: palladium on carbon; PdOAc₂: palladium (II) acetate; ppm: parts per million; psi: pounds per square inch; q: quartet; s: singlet; t: triplet; td: triplet of doublet; TEMPO: 2,2,6,6-Tetramethylpiperidine-1-oxyl; TFA: trifluoroacetic acid; THF: tetrahydrofuran.

Column chromatography was performed using 32-63 micron, 60 Å, silica gel with glass column and air pressure. MPLC was performed on an ISCO Companion instrument using pre-packaged disposable RediSep SiO₂ stationary phase at 5-100 mL/min, UV detection (190-760 nm range).

The mass spectra were recorded utilising electrospray (LC-MS; column XTerra MS C8 2.5 μm 2.1×30 mm, buffer gradient H₂O 0.1% TFA: CH₃CN+0.04% TFA, MS: micromass ZMD/ammonium acetate buffer) ionisation techniques. Alternatively, mass spectra were recorded on a Waters MS consisting of an Alliance 2795 (LC) and Waters Micromass ZQ detector at 120° C. The mass spectrometer was equipped with an electrospray ion source (ESI) operated in a positive or negative ion mode. The mass spectrometer was scanned between m/z 100-1000 with a scan time of 0.3 s.

The ¹H NMR spectra were recorded on Varian NMR Spectrometer at 400 MHz. The ¹H NMR spectra were interpreted using the processing software ACD/SpecManager version 10.02. Alternatively, they were recorded on a Bruker UltraShield Advance 400 MHz/54 mm spectrometer and processed with XWIN-NMR version 2.6 software. The chemical shifts (6) are reported in parts-per-million from a tetramethylsilane internal standard.

### Example 1

### (4-cyclobutylpiperazin-1-yl)(7-(pyridin-3-yl)-7-azaspiro[3.5]nonan-2-yl)methanone

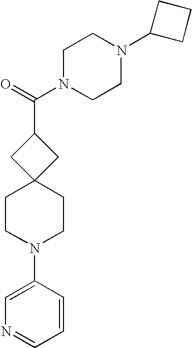

Cesium carbonate (123 mg, 0.38 mmol) was added to a solution of Intermediate 7 (100 mg, 0.34 mmol), PdOAc₂ (7.70 mg, 0.03 mmol), BINAP (42.7 mg, 0.07 mmol) and 3-bromopyridine (56.9 mg, 0.36 mmol) in toluene (2 mL). The reaction mixture was heated to 110° C. for 18 h. The room temperature cooled mixture was filtered over celite. The solvent was concentrated. The product was purified by preparative HPLC using a low pH shallow gradient method (Mobile phase: 20-40% B; A: H₂O with 0.05% TFA, B: CH₃CN, 25 min. run) on Luna 15 μm, C18, 21.2×250 mm Phenomenex reverse phase column to provide title compound (10.00 mg, 7.91%) as a solid. ¹H NMR (400 MHz, CDCl₃) δ ppm 1.58 (s, 3H) 1.63 (dd, J=6.25, 4.69 Hz, 2H) 1.66-1.79 (m, 3H) 1.80-1.94 (m, 2H) 1.97-2.10 (m, 3H) 2.12-2.21 (m, 2H) 2.27 (q, J=5.60 Hz, 3H) 2.71 (quintet, J=7.91 Hz, 1H) 3.21 (quintet, J=8.79 Hz, 1H) 3.32-3.39 (m, 2H) 3.41 (dd, J=6.64, 4.69 Hz, 2H) 3.54 (dd, J=6.25, 4.69 Hz, 2H) 3.63 (t, J=4.88 Hz, 2H) 6.53-6.60 (m, 1H) 6.65 (d, J=8.59 Hz, 1H) 7.40-7.48 (m, 1H) 8.16 (td, J=2.34, 1.17 Hz, 1H); HRMS (ESI-TOF) m/z calcd for C₂₂H₃₃N₄O 369.26489 [M+H]⁺, found 369.26533.

### Example 2

### (4-cyclobutylpiperazin-1-yl)(7-(pyridin-2-yl)-7-azaspiro[3.5]nonan-2-yl)methanone

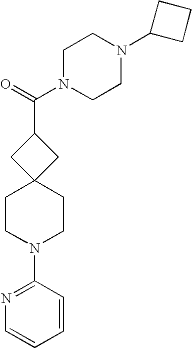

Cesium carbonate (123 mg, 0.38 mmol) was added to a solution of Intermediate 7 (100 mg, 0.34 mmol), PdOAc₂ (7.70 mg, 0.03 mmol), BINAP (42.7 mg, 0.07 mmol) and 2-bromopyridine (56.9 mg, 0.36 mmol) in toluene (2 mL). The reaction mixture was heated to 110° C. for 18 h. The room temperature cooled mixture was filtered over celite. The solvent was concentrated. The product was purified by preparative HPLC using a low pH shallow gradient method (Mobile phase: 20-40% B; A: H₂O with 0.05% TFA, B: CH₃CN, 25 min. run) on Luna 15 μm, C18, 21.2×250 mm Phenomenex reverse phase column to provide title compound (54.1 mg, 42.8%) as a solid. ¹H NMR (400 MHz, CDCl₃) δ ppm 1.62-1.75 (m, 4H) 1.75-1.81 (m, 2H) 1.81-1.94 (m, 2H) 1.98-2.10 (m, 4H) 2.12-2.21 (m, 2H) 2.23-2.32 (m, 4H) 2.71 (quintet, J=7.91 Hz, 1H) 3.05-3.14 (m, 2H) 3.14-3.26 (m, 3H) 3.33-3.40 (m, 2H) 3.58-3.68 (m, 2H) 7.09-7.15 (m, 1H) 7.16-7.21 (m, 1H) 8.06 (dd, J=4.49, 1.37 Hz, 1H) 8.31 (d, J=2.73 Hz, 1H). HRMS (ESI-TOF) m/z calcd for C₂₂H₃₃N₄O 369.26489 [M+H]⁺, found 369.26461.

### Example 3

### (4-cyclobutylpiperazin-1-yl)(7-phenyl-7-azaspiro[3.5]nonan-2-yl)methanone

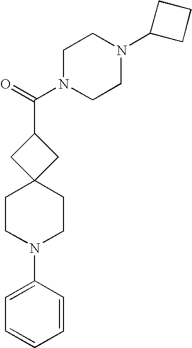

Cesium carbonate (123 mg, 0.38 mmol) was added to a solution of Intermediate 7 (100 mg, 0.34 mmol), PdOAc₂ (7.70 mg, 0.03 mmol), BINAP (42.7 mg, 0.07 mmol) and bromobenzene (56.6 mg, 0.36 mmol) in toluene (5 mL). The reaction mixture was heated to 110° C. for 18 h. The room temperature cooled mixture was filtered over celite. The solvent was concentrated. The product was purified by preparative HPLC using a low pH shallow gradient method (Mobile phase: 20-40% B; A: H₂O with 0.05% TFA, B: CH₃CN, 25 min. run) on Luna 15 μm, C18, 21.2×250 mm Phenomenex reverse phase column to provide title compound (59.0 mg, 46.8%) as a solid. ¹H NMR (400 MHz, CDCl₃) δ ppm 1.60-1.75 (m, 4H) 1.75-1.81 (m, 2H) 1.81-1.95 (m, 2H) 1.96-2.10 (m, 4H) 2.10-2.20 (m, 2H) 2.21-2.35 (m, 4H) 2.71 (dq, J=8.01, 7.75 Hz, 1H) 3.00-3.11 (m, 2H) 3.11-3.26 (m, 3H) 3.31-3.42 (m, 2H) 3.56-3.70 (m, 2H) 6.82 (t, J=7.23 Hz, 1H) 6.93 (d, J=7.81 Hz, 2H) 7.18-7.29 (m, 2H); HRMS (ESI-TOF) m/z calcd for C₂₃H₃₄N₃O 368.26964 [M+H]⁺, found 368.26981.

### Example 4

### (4-cyclobutylpiperazin-1-yl)(7-(isopropylsulfonyl)-7-azaspiro[3.5]nonan-2-yl)methanone

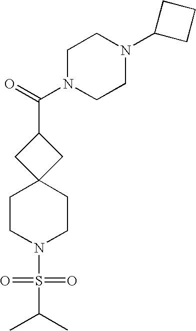

Propane-2-sulfonyl chloride (58.7 mg, 0.41 mmol) was added to a solution of Intermediate 7 (100 mg, 0.34 mmol) and Et₃N (52.1 mg, 0.51 mmol) in DCM (10 mL) at 0° C. The reaction mixture was allowed to warm to room temperature and stirred overnight. The solvent was concentrated. The product was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (43.0 mg, 31.5%) as a solid. ¹H NMR (400 MHz, CDCl₃) δ ppm 1.33 (d, J=6.64 Hz, 6H) 1.57-1.66 (m, 2H) 1.67-1.78 (m, 4H) 1.80-1.94 (m, 2H) 1.96-2.10 (m, 4H) 2.10-2.21 (m, 2H) 2.22-2.33 (m, 4H) 2.71 (quintet, J=7.71 Hz, 1H) 3.10-3.20 (m, 2H) 3.20-3.27 (m, 2H) 3.32 (ddd, J=15.82, 5.08, 4.88 Hz, 4H) 3.62 (t, J=4.69 Hz, 2H); HRMS (ESI-TOF) m/z calcd for C₂₀H₃₆N₃O₃S 398.24719 [M+H]⁺, found 398.24764.

### Example 5

### (4-cyclobutylpiperazin-1-yl)(7-(methylsulfonyl)-7-azaspiro[3.5]nonan-2-yl)methanone

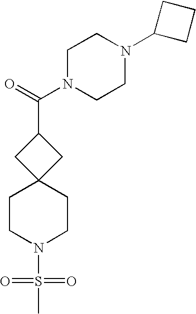

Methanesulfonyl chloride (0.032 mL, 0.41 mmol) was added to a solution of Intermediate 7 (100 mg, 0.34 mmol) and Et₃N (52.1 mg, 0.51 mmol) in DCM (10 mL) at 0° C. The reaction mixture was allowed to warm to room temperature and stirred overnight. The solvent was concentrated. The product was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (49.6 mg, 39.1%) as a solid. ¹H NMR (400 MHz, CDCl₃) δ ppm 1.59-1.72 (m, 3H) 1.72-1.80 (m, 3H) 1.79-1.94 (m, 2H) 1.95-2.10 (m, 4H) 2.09-2.20 (m, 2H) 2.22-2.34 (m, 4H) 2.66-2.74 (m, 1H) 2.76 (s, 3H) 3.05-3.15 (m, 2H) 3.14-3.26 (m, 3H) 3.30-3.38 (m, 2H) 3.62 (t, J=4.88 Hz, 2H); HRMS (ESI-TOF) m/z calcd for C₁₈H₃₂N₃O₃S 370.21589 [M+H]⁺, found 370.21603.

### Example 6

### (4-cyclobutylpiperazin-1-yl)(7-(phenylsulfonyl)-7-azaspiro[3.5]nonan-2-yl)methanone

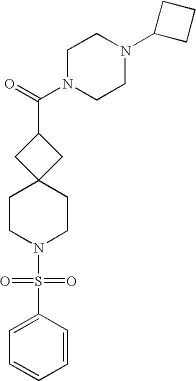

Benzenesulfonyl chloride (0.039 mL, 0.30 mmol) was added to a solution of Intermediate 7 (80 mg, 0.27 mmol) and DIEA (0.058 mL, 0.33 mmol) in DCM (8 mL). The reaction mixture was stirred for 3 days and the solvent was concentrated. The crude material was purified on preparative HPLC MS using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (44.0 mg, 37.1%) as a solid. ¹H NMR (400 MHz, CDCl₃) δ ppm 1.57-1.67 (m, 3H) 1.67-1.78 (m, 3H) 1.78-1.95 (m, 4H) 1.95-2.10 (m, 4H) 2.24 (dt, J=12.40, 5.13 Hz, 4H) 2.69 (qd, J=7.88, 7.62 Hz, 1H) 2.87-2.95 (m, 2H) 2.95-3.04 (m, 2H) 3.10 (quintet, J=8.69 Hz, 1H) 3.22-3.34 (m, 2H) 3.52-3.65 (m, 2H) 7.50-7.57 (m, 2H) 7.57-7.65 (m, 1H) 7.71-7.80 (m, 2H); HRMS (ESI-TOF) m/z calcd for C₂₃H₃₄N₃O₃S 432.23154 [M+H]⁺, found 432.23221.

### Example 7

### (4-cyclobutylpiperazin-1-yl)(7-(cyclohexanecarbonyl)-7-azaspiro[3.5]nonan-2-yl)methanone

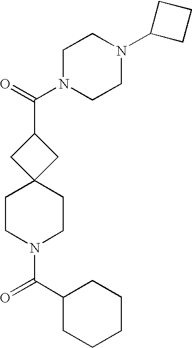

Cyclohexanecarbonyl chloride (52.6 mg, 0.36 mmol) was added to a solution of Intermediate 7 (95 mg, 0.33 mmol) and DIEA (0.085 mL, 0.49 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (44.6 mg, 34.1%) as a solid. ¹H NMR (400 MHz, CDCl₃) δ ppm 1.14-1.37 (m, 3H) 1.40-1.58 (m, 4H) 1.58-1.94 (m, 12H) 1.95-2.09 (m, 4H) 2.10-2.20 (m, 2H) 2.22-2.34 (m, 4H) 2.38-2.52 (m, 1H) 2.63-2.77 (m, 1H) 3.19 (sextet, J=8.44 Hz, 1H) 3.28-3.39 (m, 3H) 3.39-3.50 (m, 1H) 3.51-3.58 (m, 1H) 3.59-3.69 (m, 2H); HRMS (ESI-TOF) m/z calcd for C₂₄H₄₀N₃O₂ 402.31150 [M+H]% found 402.31118.

### Example 8

### (4-cyclobutylpiperazin-1-yl)(7-(3-ethylisonicotinoyl)-7-azaspiro[3.5]nonan-2-yl)methanone

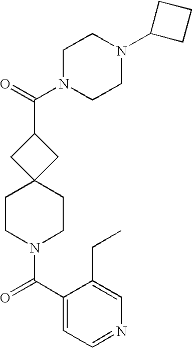

HBTU (229 mg, 0.60 mmol) and Intermediate 7 (160 mg, 0.55 mmol) were added to a solution of 3-ethylisonicotinic acid (91 mg, 0.60 mmol) and DIEA (0.192 mL, 1.10 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (67.8 mg, 29.1%) as a solid. ¹H NMR (400 MHz, CDCl₃) δ ppm 1.25 (td, J=7.52, 1.76 Hz, 3H) 1.42-1.52 (m, 1H) 1.55 (t, J=4.88 Hz, 1H) 1.60-1.79 (m, 5H) 1.79-1.95 (m, 2H) 1.97-2.09 (m, 3H) 2.09-2.21 (m, 2H) 2.22-2.35 (m, 4H) 2.53-2.78 (m, 3H) 2.99-3.30 (m, 3H) 3.30-3.41 (m, 2H) 3.52-3.72 (m, 3H) 3.72-3.91 (m, 1H) 6.98-7.10 (m, 1H) 8.48 (br. s., 1H) 8.55 (br. s., 1H); HRMS (ESI-TOF) m/z calcd for C₂₅H₃₇N₄O₂ 425.29110 [M+H]⁺, found 425.29132.

### Example 9

### (7-(5-chloronicotinoyl)-7-azaspiro[3.5]nonan-2-yl)(4-cyclobutylpiperazin-1-yl)methanone

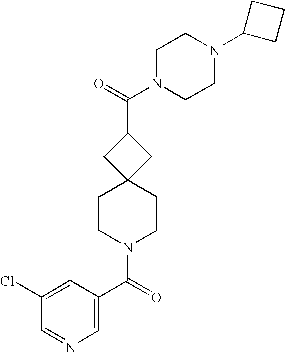

HBTU (104 mg, 0.27 mmol) and Intermediate 7 (80 mg, 0.27 mmol) were added to a solution of 5-chloronicotinic acid (43.2 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (46.6 mg, 39.4%) as a solid. ¹H NMR (400 MHz, CDCl₃) δ ppm 1.56 (br. s., 1H) 1.59-1.67 (m, 3H) 1.67-1.80 (m, 3H) 1.81-1.94 (m, 2H) 1.98-2.13 (m, 4H) 2.19 (br. s., 2H) 2.28 (q, J=5.47 Hz, 4H) 2.71 (quintet, J=7.71 Hz, 1H) 3.22 (br. s., 1H) 3.28 (br. s., 1H) 3.35 (br. s., 3H) 3.59-3.68 (m, 2H) 3.72 (br. s., 1H) 7.73 (br. s., 1H) 8.50 (s, 1H) 8.62 (d, J=2.34 Hz, 1H); HRMS (ESI-TOF) m/z calcd for C₂₃H₃₂ClN₄O₂ 431.22083 [M+H]% found 431.22123.

### Example 10

### (7-(4-chloronicotinoyl)-7-azaspiro[3.5]nonan-2-yl)(4-cyclobutylpiperazin-1-yl)methanone

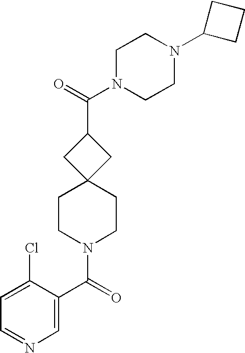

HBTU (104 mg, 0.27 mmol) and Intermediate 7 (80 mg, 0.27 mmol) were added to a solution of 4-chloronicotinic acid (43.2 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (39.9 mg, 33.7%) as a solid. ¹H NMR (400 MHz, CDCl₃) δ ppm 1.45-1.54 (m, 1H) 1.58 (dd, J=7.03, 3.91 Hz, 1H) 1.64 (br. s., 2H) 1.66-1.71 (m, 1H) 1.71-1.80 (m, 2H) 1.80-1.93 (m, 2H) 1.98-2.10 (m, 3H) 2.10-2.24 (m, 2H) 2.24-2.32 (m, 4H) 2.71 (quintet, J=7.81 Hz, 1H) 3.05-3.17 (m, 1H) 3.17-3.29 (m, 2H) 3.31-3.39 (m, 2H) 3.58-3.66 (m, 2H) 3.66-3.74 (m, 1H) 3.74-3.84 (m, 1H) 7.38 (dd, J=5.27, 2.93 Hz, 1H) 8.49 (d, J=5.86 Hz, 1H) 8.53 (dd, J=5.27, 2.15 Hz, 1H); HRMS (ESI-TOF) m/z calcd for C₂₃H₃₂ClN₄O₂ 431.22083 [M+H]⁺, found 431.22116.

### Example 11

### (4-cyclobutylpiperazin-1-yl)(7-(2,4-dimethylnicotinoyl)-7-azaspiro[3.5]nonan-2-yl)methanone

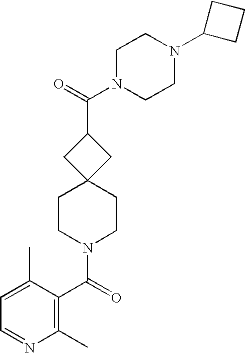

HBTU (104 mg, 0.27 mmol) and Intermediate 7 (80 mg, 0.27 mmol) were added to a solution of 2,4-dimethyl-3-pyridine carboxylic acid (41.5 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (34.8 mg, 29.9%) as a solid. ¹H NMR (400 MHz, CDCl₃) δ ppm 1.43-1.52 (m, 1H) 1.52-1.61 (m, 1H) 1.61-1.79 (m, 5H) 1.79-1.94 (m, 2H) 1.95-2.08 (m, 3H) 2.08-2.21 (m, 2H) 2.21-2.32 (m, 7H) 2.47 (d, J=3.91 Hz, 3H) 2.64-2.78 (m, 1H) 3.06 (t, J=6.05 Hz, 1H) 3.09-3.16 (m, 1H) 3.16-3.30 (m, 1H) 3.35 (dt, J=9.47, 4.83 Hz, 2H) 3.56-3.67 (m, 2H) 3.72 (q, J=5.60 Hz, 1H) 3.77-3.83 (m, 1H) 6.99 (t, 1H) 8.36 (dd, J=5.08, 3.13 Hz, 1H); HRMS (ESI-TOF) m/z calcd for C₂₅H₃₇N₄O₂ 425.29110 [M+H]% found 425.29102.

### Example 12

### (4-cyclobutylpiperazin-1-yl)(7-(6-methylnicotinoyl)-7-azaspiro[3.5]nonan-2-yl)methanone

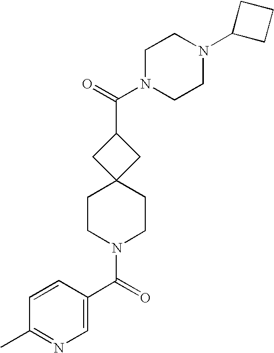

HBTU (104 mg, 0.27 mmol) and Intermediate 7 (80 mg, 0.27 mmol) were added to a solution of 6-methylnicotinic acid (37.6 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (61.9 mg, 54.9%) as a solid. ¹H NMR (400 MHz, CDCl₃) δ ppm 1.54 (br. s., 1H) 1.61 (br. s., 1H) 1.64-1.79 (m, 4H) 1.79-1.94 (m, 2H) 1.97-2.11 (m, 4H) 2.17 (br. s., 2H) 2.25-2.29 (m, 4H) 2.59 (s, 3H) 2.71 (quintet, J=7.91 Hz, 1H) 3.20 (br. s., 1H) 3.30 (br. s., 1H) 3.32-3.41 (m, 3H) 3.57-3.67 (m, 3H) 3.71 (br. s., 1H) 7.21 (d, J=7.81 Hz, 1H) 7.63 (d, J=7.42 Hz, 1H) 8.53 (d, J=1.95 Hz, 1H); HRMS (ESI-TOF) m/z calcd for C₂₄H₃₅N₄O₂ 411.27545 [M+H]% found 411.27605.

### Example 13

### (4-cyclobutylpiperazin-1-yl)(7-(2-methylnicotinoyl)-7-azaspiro[3.5]nonan-2-yl)methanone

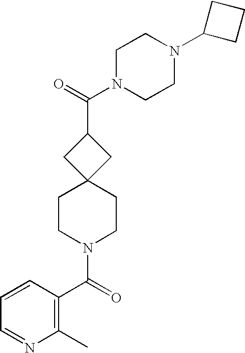

HBTU (104 mg, 0.27 mmol) and Intermediate 7 (80 mg, 0.27 mmol) were added to a solution of 2-methylnicotinic acid (37.6 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (58.3 mg, 51.7%) as a solid. ¹H NMR (400 MHz, CDCl₃) δ ppm 1.48 (dd, J=6.64, 4.69 Hz, 1H) 1.56 (dd, J=6.45, 4.49 Hz, 1H) 1.62-1.74 (m, 3H) 1.74-1.80 (m, 2H) 1.80-1.94 (m, 2H) 1.97-2.10 (m, 3H) 2.10-2.23 (m, 2H) 2.23-2.34 (m, 4H) 2.53 (d, J=3.52 Hz, 3H) 2.71 (quintet, J=7.91 Hz, 1H) 3.05-3.13 (m, 1H) 3.13-3.20 (m, 1H) 3.20-3.30 (m, 1H) 3.30-3.40 (m, 2H) 3.57-3.66 (m, 2H) 3.66-3.86 (m, 2H) 7.12-7.21 (m, 1H) 7.46 (td, J=7.42, 1.56 Hz, 1H) 8.53 (ddd, J=4.79, 2.25, 1.95 Hz, 1H); HRMS (ESI-TOF) m/z calcd for C₂₄H₃₅N₄O₂ 411.27545 [M+H]% found 411.27606.

### Example 14

### (4-cyclobutylpiperazin-1-yl)(7-(4-methylnicotinoyl)-7-azaspiro[3.5]nonan-2-yl)methanone

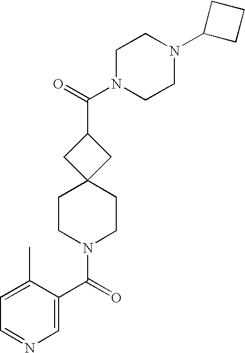

HBTU (104 mg, 0.27 mmol) and Intermediate 7 (80 mg, 0.27 mmol) were added to a solution of 4-methylnicotinic acid hydrochloride (47.7 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (46.0 mg, 40.8%) as a solid. ¹H NMR (400 MHz, CDCl₃) δ ppm 1.48 (br. s., 1H) 1.53-1.63 (m, 1H) 1.63-1.80 (m, 5H) 1.81-1.95 (m, 2H) 1.97-2.11 (m, 4H) 2.12-2.22 (m, 1H) 2.28 (br. s., 4H) 2.32 (d, J=3.12 Hz, 3H) 2.65-2.78 (m, 1H) 3.12 (br. s., 1H) 3.15-3.29 (m, 2H) 3.30-3.41 (m, 2H) 3.63 (br. s., 2H) 3.67-3.90 (m, 2H) 7.17 (t, J=4.30 Hz, 1H) 8.38 (d, J=6.25 Hz, 1H) 8.47 (d, J=4.69 Hz, 1H); HRMS (ESI-TOF) m/z calcd for C₂₄H₃₅N₄O₂ 411.27545 [M+H]⁺, found 411.27624.

### Example 15

### 6-(2-(4-cyclobutylpiperazine-1-carbonyl)-7-azaspiro[3.5]nonane-7-carbonyl)nicotinonitrile

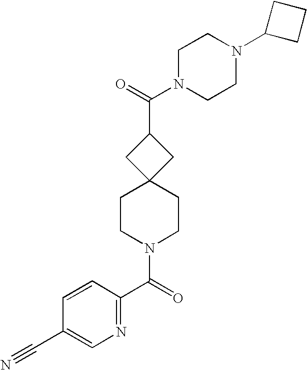

HBTU (104 mg, 0.27 mmol) and Intermediate 7 (80 mg, 0.27 mmol) were added to a solution of 5-cyanopyridine-2-carboxylic acid (40.7 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (49.7 mg, 43.0%) as a solid. ¹H NMR (400 MHz, CDCl₃) δ ppm 1.56-1.63 (m, 1H) 1.62-1.76 (m, 4H) 1.76-1.81 (m, 1H) 1.81-1.94 (m, 2H) 1.98-2.12 (m, 4H) 2.12-2.23 (m, 2H) 2.23-2.34 (m, 4H) 2.71 (quintet, J=7.71 Hz, 1H) 3.13-3.29 (m, 1H) 3.35 (ddd, J=10.84, 5.86, 5.57 Hz, 3H) 3.39-3.45 (m, 1H) 3.63 (q, J=4.95 Hz, 2H) 3.65-3.70 (m, 1H) 3.71-3.78 (m, 1H) 7.73 (dd, J=10.35, 8.40 Hz, 1H) 8.07 (dt, J=8.20, 2.34 Hz, 1H) 8.85 (s, 1H); HRMS (ESI-TOF) m/z calcd for C₂₄H₃₂N₅O₂ 422.25505 [M+H]⁺, found 422.25522.

### Example 16

### (4-cyclobutylpiperazin-1-yl)(7-(3,5-difluoropicolinoyl)-7-azaspiro[3.5]nonan-2-yl)methanone

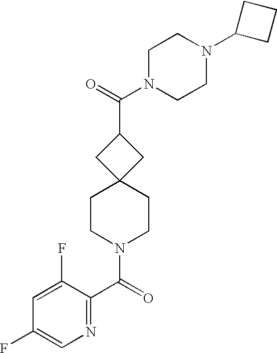

HBTU (104 mg, 0.27 mmol) and Intermediate 7 (80 mg, 0.27 mmol) were added to a solution of 3,5-difluoropyridine-2-carboxylic acid (43.7 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (56.5 mg, 47.6%) as a solid. ¹H NMR (400 MHz, CDCl₃) δ ppm 1.52-1.60 (m, 1H) 1.60-1.67 (m, 1H) 1.67-1.71 (m, 2H) 1.71-1.82 (m, 3H) 1.82-1.95 (m, 2H) 1.99-2.12 (m, 4H) 2.12-2.24 (m, 2H) 2.28 (br. s., 4H) 2.65-2.78 (m, 1H) 3.11-3.20 (m, 1H) 3.20-3.28 (m, 1H) 3.31-3.41 (m, 2H) 3.59-3.66 (m, 2H) 3.66-3.72 (m, 1H) 3.73-3.81 (m, 1H) 7.26-7.31 (m, 1H) 8.36 (t, J=2.54 Hz, 1H); HRMS (ESI-TOF) m/z calcd for C₂₃H₃₁F₂N₄O₂ 433.24096 [M+H]⁺, found 433.24127.

### Example 17

### (7-(4-chloropicolinoyl)-7-azaspiro[3.5]nonan-2-yl)(4-cyclobutylpiperazin-1-yl)methanone

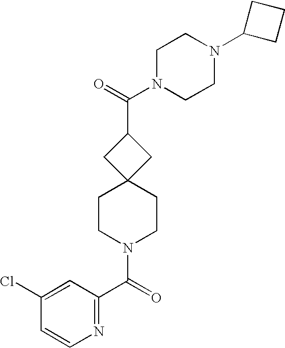

HBTU (104 mg, 0.27 mmol) and Intermediate 7 (80 mg, 0.27 mmol) were added to a solution of 4-chloropicolinic acid (43.2 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (51.9 mg, 43.9%) as a solid. ¹H NMR (400 MHz, CDCl₃) δ ppm 1.55-1.62 (m, 1H) 1.68 (dd, J=10.94, 7.03 Hz, 3H) 1.71-1.80 (m, 2H) 1.80-1.93 (m, 2H) 1.99-2.11 (m, 4H) 2.11-2.24 (m, 2H) 2.24-2.32 (m, 4H) 2.71 (quintet, J=7.71 Hz, 1H) 3.13-3.28 (m, 1H) 3.31-3.39 (m, 3H) 3.41-3.47 (m, 1H) 3.58-3.69 (m, 3H) 3.70-3.77 (m, 1H) 7.34 (dt, J=5.47, 1.95 Hz, 1H) 7.61 (dd, J=9.18, 1.76 Hz, 1H) 8.48 (dd, J=5.08, 1.95 Hz, 1H); HRMS (ESI-TOF) m/z calcd for C₂₃H₃₂ClN₄O₂ 431.22083 [M+H]% found 431.22131.

### Example 18

### (4-cyclobutylpiperazin-1-yl)(7-(6-methylpicolinoyl)-7-azaspiro[3.5]nonan-2-yl)methanone

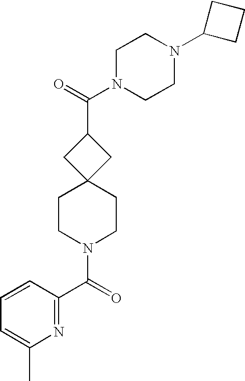

HBTU (104 mg, 0.27 mmol) and Intermediate 7 (80 mg, 0.27 mmol) were added to a solution of 6-methylpicolinic acid (37.6 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (71.2 mg, 63.2%) as a solid. ¹H NMR (400 MHz, CDCl₃) δ ppm 1.52-1.59 (m, 1H) 1.61-1.80 (m, 6H) 1.80-1.94 (m, 2H) 1.98-2.10 (m, 4H) 2.10-2.23 (m, 2H) 2.23-2.32 (m, 4H) 2.57 (d, J=2.73 Hz, 2H) 2.71 (quintet, J=7.71 Hz, 1H) 3.11-3.28 (m, 1H) 3.34 (dt, J=13.38, 5.22 Hz, 3H) 3.39-3.46 (m, 1H) 3.58-3.69 (m, 3H) 3.70-3.77 (m, 1H) 7.18 (d, J=7.42 Hz, 1H) 7.29-7.37 (m, 1H) 7.61-7.69 (m, 1H); HRMS (ESI-TOF) m/z calcd for C₂₄H₃₅N₄O₂ 411.27545 [M+H]% found 411.27559.

### Example 19

### (4-cyclobutylpiperazin-1-yl)(7-(3-methylpicolinoyl)-7-azaspiro[3.5]nonan-2-yl)methanone

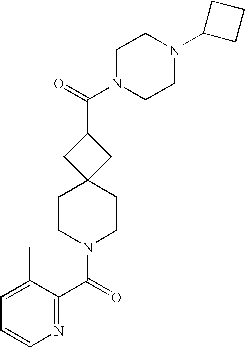

HBTU (104 mg, 0.27 mmol) and Intermediate 7 (80 mg, 0.27 mmol) were added to a solution of 3-methylpicolinic acid (37.6 mg, 0.27 mmol) and DIEA (0.144 mL, 0.82 mmol) in DMF (8 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (65.8 mg, 58.4%) as a solid. ¹H NMR (400 MHz, CDCl₃) δ ppm 1.47-1.56 (m, 1H) 1.56-1.64 (m, 1H) 1.64-1.72 (m, 2H) 1.72-1.80 (m, 2H) 1.80-1.93 (m, 2H) 1.96-2.08 (m, 4H) 2.08-2.15 (m, 1H) 2.15-2.23 (m, 1H) 2.23-2.30 (m, 4H) 2.33 (d, J=3.12 Hz, 3H) 2.65-2.77 (m, 1H) 3.02-3.11 (m, 1H) 3.11-3.17 (m, 1H) 3.17-3.29 (m, 1H) 3.34 (ddd, J=9.96, 5.27, 5.08 Hz, 2H) 3.56-3.66 (m, 2H) 3.66-3.73 (m, 1H) 3.73-3.81 (m, 1H) 7.21 (ddd, J=7.71, 4.98, 2.54 Hz, 1H) 7.50-7.60 (m, 1H) 8.42 (td, J=4.30, 1.17 Hz, 1H); HRMS (ESI-TOF) m/z calcd for C₂₄H₃₅N₄O₂ 411.27545 [M+H]% found 411.27581.

### Example 20

### (4-cyclobutylpiperazin-1-yl)(7-picolinoyl-7-azaspiro[3.5]nonan-2-yl)methanone

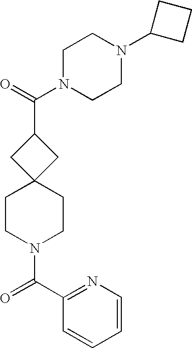

HBTU (152 mg, 0.40 mmol) and Intermediate 7 (117 mg, 0.40 mmol) were added to a solution of picolinic acid (49.4 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (81.4 mg, 51.1%) as a solid. ¹H NMR (400 MHz, DMSO-d6) δ ppm 1.33-1.44 (m, 1H) 1.44-1.52 (m, 1H) 1.52-1.70 (m, 4H) 1.70-1.84 (m, 2H) 1.86-2.06 (m, 6H) 2.15 (br. s., 4H) 2.66 (quintet, J=7.52 Hz, 1H) 3.12-3.20 (m, 1H) 3.21-3.31 (m, 4H) 3.41 (br. s., 2H) 3.46-3.55 (m, 1H) 3.55-3.65 (m, 1H) 7.39-7.56 (m, 2H) 7.82-7.97 (m, 1H) 8.57 (t, J=4.69 Hz, 1H); HRMS (ESI-TOF) m/z calcd for C₂₃H₃₃N₄O₂ 397.25980 [M+H]⁺, found 397.25926.

### Example 21

### (4-cyclobutylpiperazin-1-yl)(7-nicotinoyl-7-azaspiro[3.5]nonan-2-yl)methanone

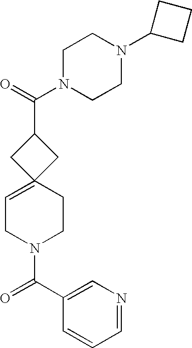

HBTU (152 mg, 0.40 mmol) and Intermediate 7 (117 mg, 0.40 mmol) were added to a solution of nicotinic acid (49.4 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (74.5 mg, 46.8%) as a solid. ¹H NMR (400 MHz, DMSO-d6) δ ppm 1.36-1.54 (m, 2H) 1.54-1.70 (m, 4H) 1.70-1.84 (m, 2H) 1.86-2.06 (m, 6H) 2.09-2.24 (m, 4H) 2.66 (quintet, J=7.71 Hz, 1H) 3.15 (br. s., 1H) 3.20-3.31 (m, 4H) 3.41 (br. s., 2H) 3.50 (br. s., 1H) 3.59 (br. s., 1H) 7.46 (dd, J=7.62, 4.88 Hz, 1H) 7.80 (d, J=7.42 Hz, 1H) 8.57 (br. s., 1H) 8.63 (dd, J=4.69, 1.56 Hz, 1H). HRMS (ESI-TOF) m/z calcd for C₂₃H₃₃N₄O₂ 397.25980 [M+H]⁺, found 397.25923.

### Example 22

### (4-cyclobutylpiperazin-1-yl)(7-isonicotinoyl-7-azaspiro[3.5]nonan-2-yl)methanone

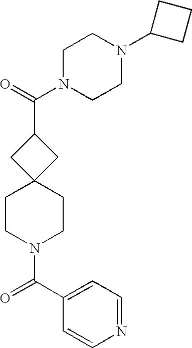

HBTU (152 mg, 0.40 mmol) and Intermediate 7 (117 mg, 0.40 mmol) were added to a solution of isonicotic acid (49.4 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (65.4 mg, 41.1%) as a solid. ¹H NMR (400 MHz, DMSO-d6) δ ppm 1.33-1.44 (m, 1H) 1.49 (dd, J=5.86, 5.08 Hz, 1H) 1.52-1.58 (m, 1H) 1.58-1.71 (m, 3H) 1.70-1.85 (m, 2H) 1.86-2.06 (m, 6H) 2.15 (d, J=3.91 Hz, 4H) 2.66 (quintet, J=7.62 Hz, 1H) 3.02-3.12 (m, 1H) 3.12-3.21 (m, 1H) 3.22-3.32 (m, 3H) 3.41 (d, J=3.52 Hz, 2H) 3.45-3.54 (m, 1H) 3.54-3.64 (m, 1H) 7.35 (t, J=6.05 Hz, 2H) 8.65 (d, J=1.17 Hz, 2H); HRMS (ESI-TOF) m/z calcd for C₂₃H₃₃N₄O₂ 397.25980 [M+H]% found 397.25885.

### Example 23

### (4-cyclobutylpiperazin-1-yl)(7-(3-methylisonicotinoyl)-7-azaspiro[3.5]nonan-2-yl)methanone

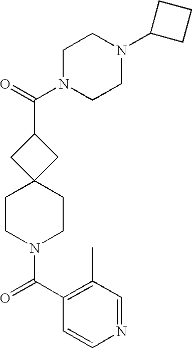

HBTU (152 mg, 0.40 mmol) and Intermediate 7 (117 mg, 0.40 mmol) were added to a solution of 3-methylisonicotinic acid (55.1 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (63.2 mg, 48.3%) as a solid. ¹H NMR (400 MHz, DMSO-d6) δ ppm 1.34 (br. s., 1H) 1.48 (t, J=5.47 Hz, 2H) 1.56-1.70 (m, 3H) 1.70-1.86 (m, 2H) 1.85-2.05 (m, 6H) 2.05-2.26 (m, 7H) 2.67 (br. s., 1H) 2.96 (t, J=5.08 Hz, 1H) 3.05 (t, J=5.08 Hz, 1H) 3.18-3.31 (m, 3H) 3.41 (br. s., 2H) 3.45-3.73 (m, 2H) 7.18 (dd, J=7.23, 4.88 Hz, 1H) 8.38-8.58 (m, 2H); HRMS (ESI-TOF) m/z calcd for C₂₄H₃₅N₄O₂ 411.27545 [M+H]⁺, found 411.27429.

### Example 24

### (4-cyclobutylpiperazin-1-yl)(7-(2-methylisonicotinoyl)-7-azaspiro[3.5]nonan-2-yl)methanone

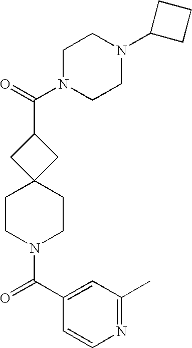

HBTU (152 mg, 0.40 mmol) and Intermediate 7 (117 mg, 0.40 mmol) were added to a solution of 2-methylisonicotinic acid (55.1 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (70.9 mg, 43.0%) as a solid. ¹H NMR (400 MHz, DMSO-d6) δ ppm 1.34-1.44 (m, 1H) 1.44-1.52 (m, 1H) 1.53-1.58 (m, 1H) 1.59-1.71 (m, 3H) 1.78 (quintet, J=8.89 Hz, 2H) 1.86-2.06 (m, 6H) 2.17 (br. s., 4H) 2.48-2.51 (m, 3H) 2.60-2.75 (m, 1H) 3.03-3.13 (m, 1H) 3.13-3.21 (m, 1H) 3.23-3.32 (m, 3H) 3.42 (br. s., 2H) 3.46-3.52 (m, 1H) 3.58 (d, J=5.08 Hz, 1H) 7.14 (t, J=5.47 Hz, 1H) 7.21 (d, J=6.64 Hz, 1H) 8.51 (t, J=4.10 Hz, 1H); HRMS (ESI-TOF) m/z calcd for C₂₄H₃₅N₄O₂ 411.27545 [M+H]⁺, found 411.27426.

### Example 25

### (4-cyclobutylpiperazin-1-yl)(7-(5-methylnicotinoyl)-7-azaspiro[3.5]nonan-2-yl)methanone

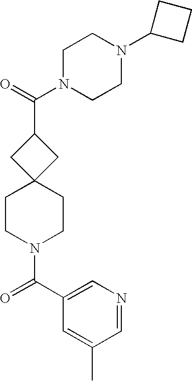

HBTU (152 mg, 0.40 mmol) and Intermediate 7 (117 mg, 0.40 mmol) were added to a solution of 5-methylisonicotinic acid (55.1 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (81.1 mg, 49.2%) as a solid. ¹H NMR (400 MHz, DMSO-d6) δ ppm 1.41 (br. s., 1H) 1.48 (br. s., 1H) 1.53-1.70 (m, 4H) 1.70-1.85 (m, 2H) 1.85-2.05 (m, 6H) 2.16 (br. s., 4H) 2.33 (s, 3H) 2.66 (quintet, J=7.71 Hz, 1H) 3.15 (br. s., 1H) 3.21-3.35 (m, 4H) 3.41 (br. s., 2H) 3.49 (br. s., 1H) 3.57 (br. s., 1H) 7.61 (br. s., 1H) 8.36 (br. s., 1H) 8.47 (s, 1H); HRMS (ESI-TOF) m/z calcd for C₂₄H₃₅N₄O₂ 411.27545 [M+H]% found 411.27527.

### Example 26

### (4-cyclobutylpiperazin-1-yl)(7-(2-methoxyisonicotinoyl)-7-azaspiro[3.5]nonan-2-yl)methanone

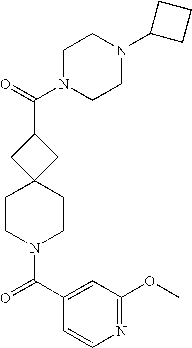

HBTU (152 mg, 0.40 mmol) and Intermediate 7 (117 mg, 0.40 mmol) were added to a solution of 2-methoxyisonicotinic acid (61.5 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (70.2 mg, 41.0%) as a solid. ¹H NMR (400 MHz, DMSO-d6) δ ppm 1.33-1.43 (m, 1H) 1.43-1.51 (m, 1H) 1.51-1.58 (m, 1H) 1.58-1.70 (m, 3H) 1.70-1.85 (m, 2H) 1.85-2.05 (m, 6H) 2.15 (br. s., 4H) 2.60-2.73 (m, 1H) 3.03-3.11 (m, 1H) 3.12-3.21 (m, 1H) 3.22-3.31 (m, 3H) 3.41 (br. s., 2H) 3.44-3.50 (m, 1H) 3.51-3.60 (m, 1H) 3.86 (s, 3H) 6.75 (d, 1H) 6.92 (t, J=5.47 Hz, 1H) 8.16-8.30 (m, 1H); HRMS (ESI-TOF) m/z calcd for C₂₄H₃₅N₄O₃ 427.27037 [M+H]% found 427.27036.

### Example 27

### 1-(2-(4-cyclobutylpiperazine-1-carbonyl)-7-azaspiro[3.5]nonan-7-yl)-2-(pyridin-3-yl)ethanone

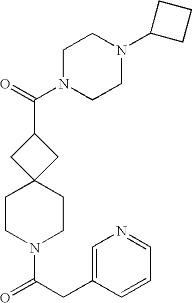

HBTU (152 mg, 0.40 mmol) and Intermediate 7 (117 mg, 0.40 mmol) were added to a solution of 2-(pyridin-3-yl)acetic acid (55.1 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (77.7 mg, 47.1%) as a solid. ¹H NMR (400 MHz, DMSO-d6) δ ppm 1.35 (dt, J=11.43, 5.81 Hz, 2H) 1.52 (q, J=5.73 Hz, 2H) 1.57-1.69 (m, 2H) 1.69-1.83 (m, 2H) 1.85-2.02 (m, 6H) 2.15 (q, J=5.47 Hz, 4H) 2.66 (quintet, J=7.71 Hz, 1H) 3.21-3.31 (m, 3H) 3.34-3.37 (m, 2H) 3.37-3.49 (m, 4H) 3.73 (d, J=9.37 Hz, 2H) 7.32 (dd, J=7.62, 4.88 Hz, 1H) 7.55-7.65 (m, 1H) 8.41 (t, J=4.88 Hz, 2H); HRMS (ESI-TOF) m/z calcd for C₂₄H₃₅N₄O₂ 411.27545 [M+H]% found 411.27526.

### Example 28

### 1-(2-(4-cyclobutylpiperazine-1-carbonyl)-7-azaspiro[3.5]nonan-7-yl)-2-(pyridin-4-yl)ethanone

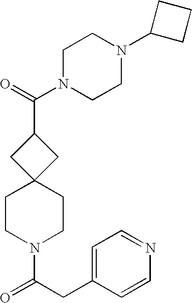

HBTU (152 mg, 0.40 mmol) and Intermediate 7 (117 mg, 0.40 mmol) were added to a solution of 2-(pyridin-4-yl)acetic acid, HCl salt (69.7 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (79.9 mg, 48.5%) as a solid. ¹H NMR (400 MHz, DMSO-d6) δ ppm 1.28-1.38 (m, 2H) 1.45-1.56 (m, 2H) 1.56-1.69 (m, 2H) 1.69-1.84 (m, 2H) 1.84-2.01 (m, 6H) 2.16 (br. s., 4H) 2.67 (br. s., 1H) 3.21-3.35 (m, 5H) 3.36-3.47 (m, 4H) 3.74 (d, J=8.98 Hz, 2H) 7.22 (t, J=4.49 Hz, 2H) 8.47 (br. s., 2H); HRMS (ESI-TOF) m/z calcd for C₂₄H₃₅N₄O₂ 411.27545 [M+H]⁺, found 411.27533.

### Example 29

### 1-(2-(4-cyclobutylpiperazine-1-carbonyl)-7-azaspiro[3.5]nonan-7-yl)-3-(pyridin-4-yl)propan-1-one

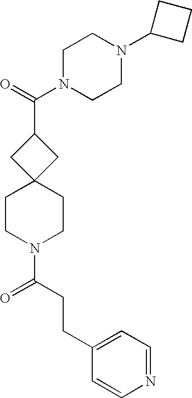

HBTU (152 mg, 0.40 mmol) and Intermediate 7 (117 mg, 0.40 mmol) were added to a solution of 3-(pyridin-4-yl)propanoic acid (60.7 mg, 40 mmol) and DIEA (0.210 mL, 1.20 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (100.4 mg, 58.9%) as a solid. ¹H NMR (400 MHz, DMSO-d6) δ ppm 1.26-1.38 (m, 2H) 1.45-1.57 (m, 2H) 1.56-1.69 (m, 2H) 1.69-1.83 (m, 2H) 1.84-2.03 (m, 6H) 2.09-2.22 (m, 4H) 2.58-2.72 (m, 3H) 2.80 (t, J=6.64 Hz, 2H) 3.20-3.31 (m, 6H) 3.36-3.47 (m, 3H) 7.18-7.32 (m, 2H) 8.43 (d, J=4.30 Hz, 2H); HRMS (ESI-TOF) m/z calcd for C₂₅H₃₇N₄O₂ 425.29110 [M+H]⁺, found 425.29010.

### Example 30

### (7-benzoyl-7-azaspiro[3.5]nonan-2-yl)(4-cyclobutylpiperazin-1-yl)methanone

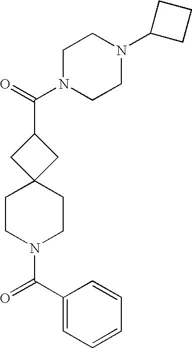

Benzoyl chloride (0.035 mL, 0.30 mmol) was added to a solution of Intermediate 7 (80 mg, 0.27 mmol) and DIEA (0.058 mL, 0.33 mmol) in DCM (8 mL). The reaction mixture was stirred for three days and the solvent was concentrated. The crude material was purified on preparative HPLC MS using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (36.5 mg, 33.6%) as a solid. ¹H NMR (400 MHz, CDCl₃) δ ppm 1.51 (br. s., 1H) 1.62-1.68 (m, 2H) 1.68-1.80 (m, 3H) 1.80-1.95 (m, 2H) 1.96-2.11 (m, 4H) 2.16 (br. s., 2H) 2.27 (q, J=5.47 Hz, 4H) 2.71 (dq, J=8.01, 7.75 Hz, 1H) 3.11-3.30 (m, 2H) 3.35 (br. s., 3H) 3.55-3.67 (m, 3H) 3.72 (br. s., 1H) 7.32-7.44 (m, 5H); HRMS (ESI-TOF) m/z calcd for C₂₄H₃₅N₃O₂ [M+H]⁺396.26455, found 396.26509.

### Example 31

### 3-(2-(4-cyclobutylpiperazine-1-carbonyl)-7-azaspiro[3.5]nonane-7-carbonyl)benzonitrile

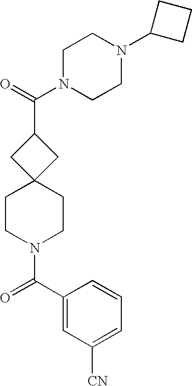

3-Cyanobenzoyl chloride (101 mg, 0.61 mmol) was added to a solution of Intermediate 7 (161 mg, 0.55 mmol) and DIEA (0.145 mL, 0.83 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (19.10 mg, 8.22%) as a solid. ¹H NMR (400 MHz, CDCl₃) δ ppm 1.54 (br. s., 1H) 1.61 (br. s., 1H) 1.64-1.81 (m, 4H) 1.81-1.96 (m, 2H) 1.97-2.12 (m, 5H) 2.18 (br. s., 2H) 2.29 (d, J=4.69 Hz, 4H) 2.72 (quintet, J=7.91 Hz, 1H) 3.23 (br. s., 2H) 3.30 (br. s., 1H) 3.36 (br. s., 2H) 3.63 (br. s., 2H) 3.72 (br. s., 1H) 7.29-7.39 (m, 1H) 7.54 (t, J=7.62 Hz, 1H) 7.62 (d, J=7.03 Hz, 1H) 7.65-7.74 (m, 1H); HRMS (ESI-TOF) m/z calcd for C₂₅H₃₃N₄O₂ [M+H]⁺421.25980, found 421.25958.

### Example 32

### 2-(4-cyclobutylpiperazine-1-carbonyl)-N-phenyl-7-azaspiro[3.5]nonane-7-carboxamide

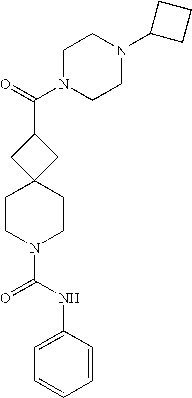

Phenyl isocyanate (33.0 μl, 0.30 mmol) was added to a solution of Intermediate 7 (80 mg, 0.27 mmol) in DCM. The reaction mixture was stirred for 3 days and the solvent was concentrated. The crude material was purified on preparative HPLC MS using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (34.5 mg, 30.6%) as a solid. ¹H NMR (400 MHz, CDCl₃) δ ppm 1.57-1.64 (m, 2H) 1.64-1.80 (m, 4H) 1.80-1.95 (m, 2H) 1.97-2.11 (m, 4H) 2.11-2.22 (m, 2H) 2.23-2.36 (m, 4H) 2.72 (dq, J=8.01, 7.75 Hz, 1H) 3.21 (quintet, J=8.69 Hz, 1H) 3.35 (q, J=5.60 Hz, 4H) 3.41-3.52 (m, 2H) 3.57-3.69 (m, 2H) 6.37 (s, 1H) 7.02 (t, J=7.23 Hz, 1H) 7.24-7.31 (m, 2H) 7.31-7.40 (m, 2H); HRMS (ESI-TOF) m/z calcd for C₂₄H₃₅N₄O₂ 411.27545 [M+H]⁺, found 411.27612.

### Example 33

### (4-isopropylpiperazin-1-yl)(7-(methylsulfonyl)-7-azaspiro[3.5]nonan-2-yl)methanone

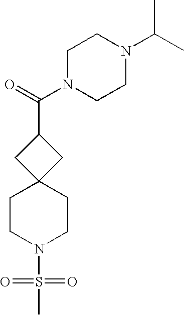

Methanesulfonyl chloride (0.033 mL, 0.43 mmol) was added to a solution of Intermediate 9 (100 mg, 0.36 mmol) and Et₃N (0.075 mL, 0.54 mmol) in DCM (12 mL) at 0° C. The reaction mixture was stirred for 18 h and the solvent was concentrated. The product was purified on preparative reverse-phase HPLC using a low pH shallow gradient method (Mobile phase: 30-50% B; A: H₂O with 0.05% TFA, B: CH₃CN, 25 min. run) on Luna 15 μm, C18, 21.2×250 mm Phenomenex reverse phase column to provide title compound (30.3 mg, 23.68%) as a solid. ¹H NMR (400 MHz, CDCl₃) δ ppm 1.04 (d, J=6.25 Hz, 6H) 1.64-1.72 (m, 2H) 1.72-1.80 (m, 2H) 1.96-2.07 (m, 2H) 2.10-2.20 (m, 2H) 2.40-2.52 (m, 4H) 2.70 (quintet, J=6.64 Hz, 1H) 2.76 (s, 3H) 3.07-3.14 (m, 2H) 3.15-3.25 (m, 3H) 3.28-3.38 (m, 2H) 3.59-3.63 (m, 2H); HRMS (ESI-TOF) m/z calcd for C₁₇H₃₂N₃O₃S 358.21589 [M+H]⁺, found 358.21573.

### Example 34

### (4-isopropylpiperazin-1-yl)(7-(2-methylisonicotinoyl)-7-azaspiro[3.5]nonan-2-yl)methanone

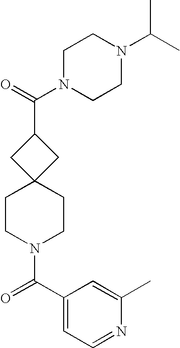

HBTU (204 mg, 0.54 mmol) and Intermediate 9 (100 mg, 0.36 mmol) were added to a solution of 2-methylisonicotinic acid (73.6 mg, 0.54 mmol) and DIEA (0.125 mL, 0.72 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (49.7 mg, 34.8%) as a solid. ¹H NMR (400 MHz, CDCl₃) δ ppm 1.04 (d, J=6.64 Hz, 6H) 1.52 (br. s., 1H) 1.59 (br. s., 1H) 1.67 (br. s., 1H) 1.76 (br. s., 1H) 1.97-2.12 (m, 2H) 2.12-2.27 (m, 2H) 2.47 (br. s., 4H) 2.59 (s, 3H) 2.70 (dt, J=12.89, 6.45 Hz, 1H) 3.13-3.31 (m, 3H) 3.35 (br. s., 2H) 3.62 (br. s., 3H) 3.71 (br. s., 1H) 7.04 (br. s., 1H) 7.13 (d, J=5.08 Hz, 1H) 8.55 (d, J=3.91 Hz, 1H); HRMS (ESI-TOF) m/z calcd for C₂₃H₃₅N₄O₂ 399.27545 [M+H]⁺, found 399.27528.

### Example 35

### (7-isonicotinoyl-7-azaspiro[3.5]nonan-2-yl)(4-isopropylpiperazin-1-yl)methanone

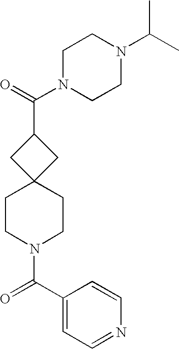

HBTU (204 mg, 0.54 mmol) and Intermediate 9 (100 mg, 0.36 mmol) were added to a solution of isonicotinic acid (66.1 mg, 0.54 mmol) and DIEA (0.125 mL, 0.72 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (39.7 mg, 28.8%) as a solid. ¹H NMR (400 MHz, CDCl₃) δ ppm 1.04 (d, J=6.30 Hz, 6H) 1.47-1.56 (m, 1H) 1.56-1.64 (m, 1H) 1.64-1.72 (m, 1H) 1.72-1.81 (m, 1H) 1.98-2.12 (m, 2H) 2.12-2.28 (m, 2H) 2.37-2.56 (m, 4H) 2.70 (dt, J=12.99, 6.59 Hz, 1H) 3.13-3.24 (m, 2H) 3.24-3.31 (m, 1H) 3.31-3.41 (m, 2H) 3.56-3.68 (m, 3H) 3.68-3.78 (m, 1H) 7.18-7.31 (m, 2H) 8.68 (d, J=4.69 Hz, 2H); HRMS (ESI-TOF) m/z calcd for C₂₂H₃₃N₄O₂ 385.25980 [M+H]⁺, found 385.25977.

### Example 36

### (4-isopropylpiperazin-1-yl)(7-nicotinoyl-7-azaspiro[3.5]nonan-2-yl)methanone

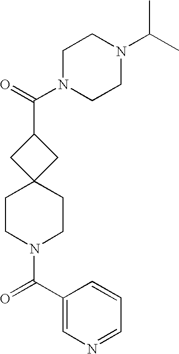

HBTU (204 mg, 0.54 mmol) and Intermediate 9 (100 mg, 0.36 mmol) were added to a solution of nicotinic acid (66.1 mg, 0.54 mmol) and DIEA (0.125 mL, 0.72 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (48.5 mg, 35.2%) as a solid. ¹H NMR (400 MHz, CDCl₃) δ ppm 1.04 (d, J=6.70 Hz, 6H) 1.63 (br. s., 3H) 1.77 (br. s., 1H) 2.07 (br. s., 2H) 2.17 (br. s., 2H) 2.47 (q, J=5.08 Hz, 4H) 2.70 (quintet, J=6.54 Hz, 1H) 3.14-3.31 (m, 2H) 3.35 (br. s., 3H) 3.55-3.69 (m, 3H) 3.73 (br. s., 1H) 7.36 (dd, J=7.42, 5.08 Hz, 1H) 7.73 (d, J=6.64 Hz, 1H) 8.65 (td, J=4.10, 1.56 Hz, 2H); HRMS (ESI-TOF) m/z calcd for C₂₂H₃₂N₄O₂ 385.25980 [M+H]⁺, found 385.25991.

### Example 37

### 2-(4-isopropylpiperazine-1-carbonyl)-N,N-dimethyl-7-azaspiro[3.5]nonane-7-carboxamide

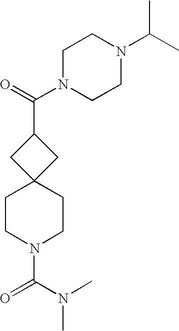

Dimethylcarbamic chloride (46.2 mg, 0.43 mmol) was added to a solution of Intermediate 9 (100 mg, 0.36 mmol) and Et₃N (0.075 mL, 0.54 mmol) in DCM (12 mL) at 0° C. The reaction mixture was stirred for 18 h and the solvent was concentrated. The product was purified on preparative reverse-phase HPLC using a low pH shallow gradient method (Mobile phase: 30-50% B; A: H₂O with 0.05% TFA, B: CH₃CN, 25 min. run) on Luna 15 μm, C18, 21.2×250 mm Phenomenex reverse phase column to provide title compound (56.7 mg, 45.2%) as a solid. ¹H NMR (400 MHz, CDCl₃) δ ppm 1.04 (d, J=6.64 Hz, 6H) 1.49-1.59 (m, 2H) 1.60-1.68 (m, 2H) 1.95-2.06 (m, 2H) 2.08-2.17 (m, 2H) 2.46 (q, J=5.08 Hz, 4H) 2.70 (dt, J=12.99, 6.59 Hz, 1H) 2.80 (s, 6H) 3.03-3.11 (m, 2H) 3.12-3.25 (m, 3H) 3.30-3.39 (m, 2H) 3.56-3.66 (m, 2H); HRMS (ESI-TOF) m/z calcd for C₁₉H₃₅N₄O₂ 351.27545 [M+H]⁺, found 351.27564.

### Example 38

### (4-cyclobutyl-1,4-diazepan-1-yl)(7-(4-fluorophenylsulfonyl)-7-azaspiro[3.5]nonan-2-yl)methanone

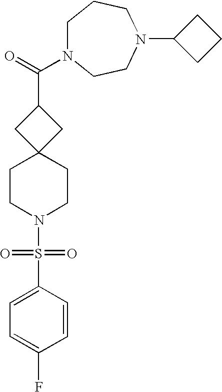

4-Fluorobenzene-1-sulfonyl chloride (66.6 mg, 0.34 mmol) was added to a solution of Intermediate 11 (95 mg, 0.31 mmol) and DIEA (0.081 mL, 0.47 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (71.3 mg, 49.5%) as a solid. ¹H NMR (400 MHz, CDCl₃) δ ppm 1.55-1.62 (m, 1H) 1.62-1.70 (m, 4H) 1.70-1.77 (m, 2H) 1.77-1.87 (m, 4H) 1.87-1.94 (m, 1H) 1.97-2.11 (m, 4H) 2.33-2.41 (m, 2H) 2.44 (dt, J=7.42, 4.88 Hz, 2H) 2.76-2.88 (m, 1H) 2.88-2.95 (m, 2H) 2.95-3.03 (m, 2H) 3.13 (qd, J=8.66, 8.40 Hz, 1H) 3.36 (t, J=6.25 Hz, 2H) 3.53-3.64 (m, 2H) 7.22 (t, J=8.59 Hz, 2H) 7.73-7.81 (m, 2H); HRMS (ESI-TOF) m/z calcd for C₂₄H₃₅FN₃O₃S 464.23777 [M+H]⁺, found 464.23859.

### Example 39

### (4-cyclobutyl-1,4-diazepan-1-yl)(7-(3-fluorophenylsulfonyl)-7-azaspiro[3.5]nonan-2-yl)methanone

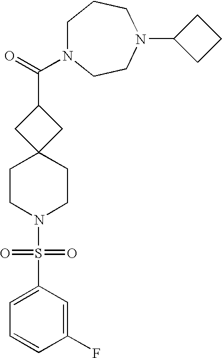

3-Fluorobenzene-1-sulfonyl chloride (66.6 mg, 0.34 mmol) was added to a solution of Intermediate 11 (95 mg, 0.31 mmol) and DIEA (0.081 mL, 0.47 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (34.7 mg, 24.07%) as a solid. ¹H NMR (400 MHz, CDCl₃) δ ppm 1.55-1.62 (m, 1H) 1.62-1.70 (m, 4H) 1.70-1.77 (m, 2H) 1.77-1.87 (m, 4H) 1.87-1.94 (m, 1H) 1.97-2.11 (m, 4H) 2.33-2.41 (m, 2H) 2.44 (dt, J=7.42, 4.88 Hz, 2H) 2.76-2.88 (m, 1H) 2.88-2.95 (m, 2H) 2.95-3.03 (m, 2H) 3.13 (qd, J=8.66, 8.40 Hz, 1H) 3.36 (t, J=6.25 Hz, 2H) 3.53-3.64 (m, 2H) 7.22 (t, J=8.59 Hz, 2H) 7.73-7.81 (m, 2H); HRMS (ESI-TOF) m/z calcd for C₂₄H₃₅FN₃O₃S 464.23777 [M+H]⁺, found 464.23782.

### Example 40

### (4-cyclobutyl-1,4-diazepan-1-yl)(7-(2-fluorophenylsulfonyl)-7-azaspiro[3.5]nonan-2-yl)methanone

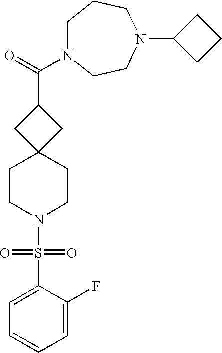

2-Fluorobenzene-1-sulfonyl chloride (66.6 mg, 0.34 mmol) was added to a solution of Intermediate 11 (95 mg, 0.31 mmol) and DIEA (0.081 mL, 0.47 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (95 mg, 65.9%) as a solid. ¹H NMR (400 MHz, CDCl₃) δ ppm 1.54-1.62 (m, 1H) 1.62-1.67 (m, 3H) 1.67-1.76 (m, 2H) 1.77-1.89 (m, 4H) 1.89-1.98 (m, 2H) 1.98-2.14 (m, 4H) 2.32-2.42 (m, 2H) 2.42-2.54 (m, 2H) 2.83 (quintet, J=7.71 Hz, 1H) 3.03-3.14 (m, 2H) 3.14-3.24 (m, 3H) 3.37 (t, J=6.25 Hz, 2H) 3.52-3.66 (m, 2H) 7.17-7.25 (m, 1H) 7.25-7.31 (m, 1H) 7.52-7.62 (m, 1H) 7.84 (td, J=7.42, 1.95 Hz, 1H); HRMS (ESI-TOF) m/z calcd for C₂₄H₃₅FN₃O₃S 464.23777 [M+H]⁺, found 464.23789.

### Example 41

### (4-cyclobutyl-1,4-diazepan-1-yl)(7-(phenylsulfonyl)-7-azaspiro[3.5]nonan-2-yl)methanone

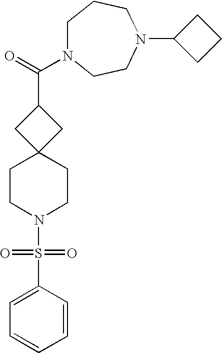

Benzenesulfonyl chloride (0.044 mL, 0.34 mmol) was added to a solution of Intermediate 11 (95 mg, 0.31 mmol) and DIEA (0.081 mL, 0.47 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (60.6 mg, 43.7%) as a solid. ¹H NMR (400 MHz, CDCl₃) δ ppm 1.53-1.62 (m, 1H) 1.62-1.69 (m, 3H) 1.69-1.75 (m, 3H) 1.75-1.93 (m, 5H) 1.96-2.10 (m, 4H) 2.31-2.41 (m, 2H) 2.41-2.50 (m, 2H) 2.82 (quintet, J=7.71 Hz, 1H) 2.88-2.95 (m, 2H) 2.95-3.04 (m, 2H) 3.11 (quintet, J=8.69 Hz, 1H) 3.29-3.40 (m, 2H) 3.51-3.65 (m, 2H) 7.50-7.57 (m, 2H) 7.57-7.64 (m, 1H) 7.71-7.80 (m, 2H); HRMS (ESI-TOF) m/z calcd for C₂₄H₃₆N₃O₃S 446.24719 [M+H]% found 446.24713.

### Example 42

### (4-cyclobutyl-1,4-diazepan-1-yl)(7-picolinoyl-7-azaspiro[3.5]nonan-2-yl)methanone

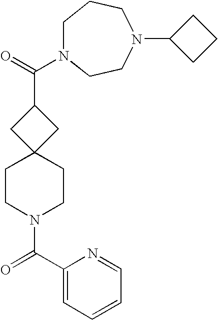

HBTU (140 mg, 0.37 mmol) and Intermediate 11 (75 mg, 0.25 mmol) were added to a solution of picolinic acid (45.3 mg, 0.37 mmol) and DIEA (0.086 mL, 0.49 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (54.3 mg, 53.9%) as a solid. ¹H NMR (400 MHz, CDCl₃) δ ppm 1.49-1.60 (m, 2H) 1.60-1.72 (m, 3H) 1.72-1.92 (m, 5H) 1.92-2.10 (m, 5H) 2.10-2.26 (m, 1H) 2.39 (d, J=4.69 Hz, 2H) 2.46 (d, J=3.52 Hz, 2H) 2.83 (quintet, J=7.71 Hz, 1H) 3.09-3.30 (m, 1H) 3.30-3.37 (m, 1H) 3.37-3.49 (m, 3H) 3.53-3.69 (m, 3H) 3.69-3.78 (m, 1H) 7.09-7.26 (m, 1H) 7.48-7.62 (m, 1H) 7.77 (t, J=7.62 Hz, 1H) 8.57 (d, J=3.91 Hz, 1H); HRMS (ESI-TOF) m/z calcd for C₂₄H₃₅N₄O₂ 411.27545 [M+H]⁺, found 411.27600.

### Example 43

### (4-cyclobutyl-1,4-diazepan-1-yl)(7-(2-methylisonicotinoyl)-7-azaspiro[3.5]nonan-2-yl)methanone

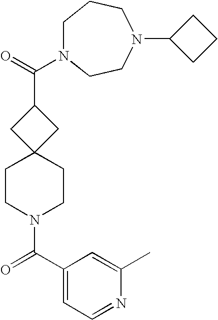

HBTU (140 mg, 0.37 mmol) and Intermediate 11 (75 mg, 0.25 mmol) were added to a solution of 2-methylisonicotinic acid (50.5 mg, 0.37 mmol) and DIEA (0.086 mL, 0.49 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (25.6 mg, 24.56%) as a solid. ¹H NMR (400 MHz, CDCl₃) δ ppm 1.52 (br. s., 1H) 1.55-1.72 (m, 3H) 1.72-1.93 (m, 6H) 1.94-2.11 (m, 4H) 2.11-2.27 (m, 2H) 2.33-2.44 (m, 2H) 2.47 (br. s., 2H) 2.58 (s, 3H) 2.84 (quintet, J=7.62 Hz, 1H) 3.20 (d, J=4.69 Hz, 2H) 3.25 (d, J=8.98 Hz, 1H) 3.41 (d, J=5.47 Hz, 2H) 3.62 (br. s., 3H) 3.71 (br. s., 1H) 7.04 (br. s., 1H) 7.12 (d, J=4.69 Hz, 1H) 8.54 (d, J=4.69 Hz, 1H); HRMS (ESI-TOF) m/z calcd for C₂₅H₃₇N₄O₂ 425.29110 [M+H]⁺, found 425.29164.

### Example 44

### (4-cyclobutyl-1,4-diazepan-1-yl)(7-(3-methylisonicotinoyl)-7-azaspiro[3.5]nonan-2-yl)methanone

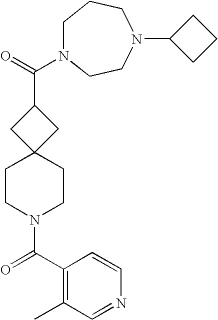

HBTU (140 mg, 0.37 mmol) and Intermediate 11 (75 mg, 0.25 mmol) were added to a solution of 3-methylisonicotinic acid (50.5 mg, 0.37 mmol) and DIEA (0.086 mL, 0.49 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (42.4 mg, 40.7%) as a solid. ¹H NMR (400 MHz, CDCl₃) δ ppm 1.47 (d, J=3.12 Hz, 1H) 1.51-1.71 (m, 4H) 1.71-1.89 (m, 5H) 1.94-2.23 (m, 7H) 2.27 (d, J=2.73 Hz, 3H) 2.33-2.42 (m, 2H) 2.45 (d, J=2.73 Hz, 2H) 2.82 (quintet, J=7.71 Hz, 1H) 3.05 (t, J=5.47 Hz, 1H) 3.12 (t, J=5.27 Hz, 1H) 3.14-3.29 (m, 1H) 3.33-3.45 (m, 2H) 3.50-3.64 (m, 2H) 3.65-3.86 (m, 1H) 6.96-7.26 (m, 1H) 8.47 (d, J=9.77 Hz, 2H); HRMS (ESI-TOF) m/z calcd for C₂₅H₃₇N₄O₂ 425.29110 [M+H]⁺, found 425.29114.

### Example 45

### (4-cyclobutyl-1,4-diazepan-1-yl)(7-isonicotinoyl-7-azaspiro[3.5]nonan-2-yl)methanone

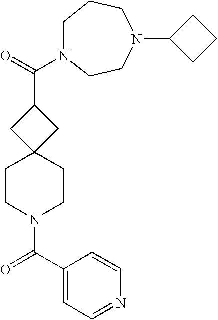

HBTU (140 mg, 0.37 mmol) and Intermediate 11 (75 mg, 0.25 mmol) were added to a solution of isonicotinic acid (45.3 mg, 0.37 mmol) and DIEA (0.086 mL, 0.49 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (31.2 mg, 31.0%) as a solid. ¹H NMR (400 MHz, CDCl₃) δ ppm 1.53 (br. s., 1H) 1.56-1.72 (m, 3H) 1.73-1.94 (m, 6H) 1.94-2.12 (m, 4H) 2.12-2.29 (m, 2H) 2.35-2.44 (m, 2H) 2.47 (br. s., 2H) 2.84 (quintet, J=7.71 Hz, 1H) 3.21 (d, J=4.69 Hz, 2H) 3.23-3.33 (m, 1H) 3.40 (d, J=5.47 Hz, 2H) 3.62 (d, J=4.30 Hz, 3H) 3.72 (br. s., 1H) 7.26 (d, J=6.25 Hz, 2H) 8.68 (d, J=4.30 Hz, 2H); HRMS (ESI-TOF) m/z calcd for C₂₄H₃₅N₄O₂ 411.27545+H]⁺, found 411.27600.

### Example 46

### (4-cyclobutyl-1,4-diazepan-1-yl)(7-(5-methylnicotinoyl)-7-azaspiro[3.5]nonan-2-yl)methanone

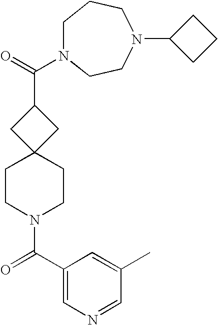

HBTU (102 mg, 0.27 mmol) and Intermediate 11 (75 mg, 0.25 mmol) were added to a solution of 5-methylnicotinic acid (33.7 mg, 0.25 mmol) and DIEA (0.086 mL, 0.49 mmol) in DMF (6 mL). The reaction mixture was stirred for 3 days and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (64.6 mg, 62.0%) as a solid. ¹H NMR (400 MHz, CDCl₃) δ ppm 1.50-1.72 (m, 5H) 1.72-1.93 (m, 5H) 1.96-2.13 (m, 4H) 2.18 (br. s., 2H) 2.37 (s, 3H) 2.39-2.44 (m, 2H) 2.44-2.53 (m, 2H) 2.77-2.91 (m, 1H) 3.13-3.32 (m, 2H) 3.36 (br. s., 1H) 3.42 (d, J=6.25 Hz, 2H) 3.55-3.68 (m, 3H) 3.73 (br. s., 1H) 7.54 (br. s., 1H) 8.43 (s, 1H) 8.48 (s, 1H); HRMS (ESI-TOF) m/z calcd for C₂₅H₃₇N₄O₂ 425.29110 [M+H]⁺, found 425.29203.

### Example 47

### (4-cyclobutyl-1,4-diazepan-1-yl)(7-nicotinoyl-7-azaspiro[3.5]nonan-2-yl)methanone

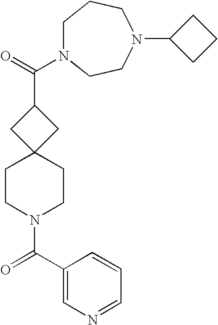

HBTU (102 mg, 0.27 mmol) and Intermediate 11 (75 mg, 0.25 mmol) were added to a solution of nicotinic acid (30.2 mg, 0.25 mmol) and DIEA (38.1 mg, 0.29 mmol) in DMF (6 mL). The reaction mixture was stirred for 3 days and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (50.6 mg, 50.2%) as a solid. ¹H NMR (400 MHz, CDCl₃) δ ppm 1.50-1.74 (m, 5H) 1.74-1.94 (m, 5H) 1.95-2.13 (m, 4H) 2.18 (br. s., 2H) 2.36-2.45 (m, 2H) 2.45-2.53 (m, 2H) 2.85 (quintet, 1H) 3.15-3.32 (m, 2H) 3.36 (br. s., 1H) 3.41 (br. s., 2H) 3.56-3.69 (m, 3H) 3.74 (br. s., 1H) 7.36 (dd, J=7.62, 4.88 Hz, 1H) 7.73 (d, J=6.64 Hz, 1H) 8.60-8.70 (m, 2H); HRMS (ESI-TOF) m/z calcd for C₂₄H₃₅N₄O₂ 411.27545 [M+H]⁺, found 411.27608.

### Example 48

### (7-benzoyl-7-azaspiro[3.5]nonan-2-yl)(4-cyclobutyl-1,4-diazepan-1-yl)methanone

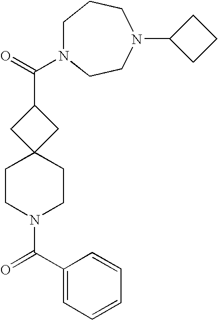

Benzoyl chloride (0.040 mL, 0.34 mmol) was added to a solution of Intermediate 11 (95 mg, 0.31 mmol) and DIEA (0.081 mL, 0.47 mmol) in DCM (10 mL). The reaction mixture was stirred overnight and the solvent was concentrated. The crude material was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (82 mg, 64.3%) as a solid. ¹H NMR (400 MHz, CDCl₃) δ ppm 1.53 (br. s., 1H) 1.56-1.72 (m, 4H) 1.72-1.95 (m, 5H) 1.96-2.11 (m, 4H) 2.17 (br. s., 2H) 2.35-2.45 (m, 2H) 2.45-2.54 (m, 2H) 2.76-2.91 (m, 1H) 3.26 (br. s., 2H) 3.34 (br. s., 1H) 3.42 (t, J=5.86 Hz, 2H) 3.55-3.68 (m, 3H) 3.72 (br. s., 1H) 7.31-7.49 (m, 5H); HRMS (ESI-TOF) m/z calcd for C₂₅H₃₆N₃O₂ 410.28020 [M+H]% found 410.27975.

### Example 49

### 4-(2-(4-cyclobutyl-1,4-diazepane-1-carbonyl)-7-azaspiro[3.5]nonane-7-carbonyl)benzonitrile

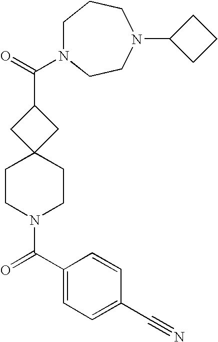

HBTU (137 mg, 0.36 mmol) and Intermediate 11 (100 mg, 0.33 mmol) were added to a solution of 4-cyanobenzoic acid (48.2 mg, 0.33 mmol) and DIEA (50.8 mg, 0.39 mmol) in DMF (10 mL). The reaction mixture was stirred overnight and the solvent was concentrated. The crude material was purified on preparative HPLC MS using the long high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (59.1 mg, 41.5%) as a solid. ¹H NMR (400 MHz, CDCl₃) δ ppm 1.56-1.74 (m, 5H) 1.74-1.93 (m, 5H) 1.96-2.12 (m, 4H) 2.12-2.29 (m, 2H) 2.34-2.44 (m, 2H) 2.48 (br. s., 2H) 2.76-2.92 (m, 1H) 3.12-3.34 (m, 3H) 3.42 (br. s., 2H) 3.53-3.68 (m, 3H) 3.73 (br. s., 1H) 7.48 (d, J=7.81 Hz, 2H) 7.71 (d, J=7.81 Hz, 2H); HRMS (ESI-TOF) m/z calcd for C₂₆H₃₅N₄O₂ 435.27545 [M+H]⁺, found 435.27502.

### Example 50

### 3-(2-(4-cyclobutyl-1,4-diazepane-1-carbonyl)-7-azaspiro[3.5]nonane-7-carbonyl)benzonitrile

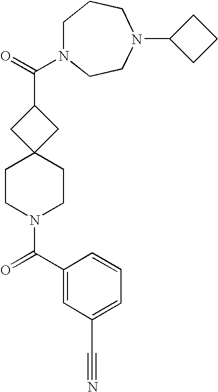

HBTU (137 mg, 0.36 mmol) and Intermediate 11 (100 mg, 0.33 mmol) were added to a solution of 3-cyanobenzoic acid (48.2 mg, 0.33 mmol) and DIEA (50.8 mg, 0.39 mmol) in DMF (10 mL). The reaction mixture was stirred overnight and the solvent was concentrated. The crude material was purified on preparative HPLC MS using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (46.2 mg, 32.5%) as a solid. ¹H NMR (400 MHz, CDCl₃) δ ppm 1.44-1.75 (m, 5H) 1.75-1.94 (m, 5H) 1.95-2.13 (m, 4H) 2.19 (br. s., 2H) 2.35-2.45 (m, 2H) 2.45-2.54 (m, 2H) 2.85 (quintet, J=7.62 Hz, 1H) 3.13-3.36 (m, 3H) 3.42 (d, J=5.47 Hz, 2H) 3.54-3.68 (m, 3H) 3.72 (br. s., 1H) 7.49-7.57 (m, 1H) 7.62 (d, J=6.25 Hz, 1H) 7.66-7.77 (m, 2H); HRMS (ESI-TOF) m/z calcd for C₂₆H₃₅N₄O₂ 435.27545 [M+H]⁺, found 435.27553.

### Example 51

### 2-(2-(4-cyclobutyl-1,4-diazepane-1-carbonyl)-7-azaspiro[3.5]nonane-7-carbonyl)benzonitrile

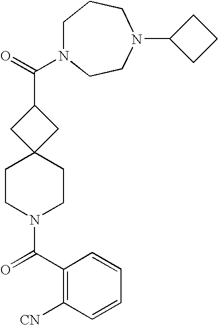

A mixture of Intermediate 11 (95 mg, 0.31 mmol), DIEA (0.068 mL, 0.39 mmol), 2-cyanobenzoic acid (38.1 mg, 0.26 mmol), EDC (59.6 mg, 0.31 mmol) and HOBT (47.6 mg, 0.31 mmol) in DMF (6.152 mL) was stirred for 18 h. The solvent was concentrated and the product was purified on preparative HPLC using the long high pH shallow gradient method (Mobile phase: 30-50% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (32.4 mg, 28.8%) as a solid. ¹H NMR (400 MHz, CDCl₃) δ ppm 1.51-1.75 (m, 5H) 1.76-1.93 (m, 5H) 1.97-2.12 (m, 4H) 2.12-2.29 (m, 2H) 2.36-2.44 (m, 2H) 2.48 (br. s., 2H) 2.85 (quintet, J=7.91 Hz, 1H) 3.16 (t, J=5.66 Hz, 1H) 3.19-3.31 (m, 2H) 3.36-3.47 (m, 2H) 3.55-3.67 (m, 2H) 3.67-3.75 (m, 1H) 3.75-3.84 (m, 1H) 7.44 (t, J=7.81 Hz, 1H) 7.51 (t, J=7.81 Hz, 1H) 7.65 (t, J=7.81 Hz, 1H) 7.71 (d, J=7.81 Hz, 1H); HRMS (ESI-TOF) m/z calcd for C₂₆H₃₅N₄O₂ 435.27545 [M+H]⁺, found 435.27503.

### Example 52

### (4-isopropyl-1,4-diazepan-1-yl)(7-(methylsulfonyl)-7-azaspiro[3.5]nonan-2-yl)methanone

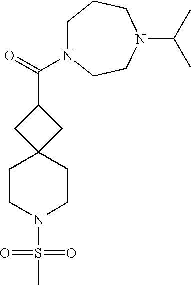

Methanesulfonyl chloride (0.038 mL, 0.49 mmol) was added to a solution of Intermediate 13 (120 mg, 0.41 mmol) and Et₃N (0.085 mL, 0.61 mmol) in DCM (12 mL) at 0° C. The reaction mixture was stirred for 18 h and the solvent was concentrated. The product was purified on preparative reverse-phase HPLC using a low pH shallow gradient method (Mobile phase: 30-50% B; A: H₂O with 0.05% TFA, B: CH₃CN, 25 min. run) on Luna 15 μm, C18, 21.2×250 mm Phenomenex reverse phase column to provide title compound (90 mg, 59.0%) as a solid. ¹H NMR (400 MHz, CDCl₃) δ ppm 0.99 (dd, J=6.64, 1.17 Hz, 6H) 1.65-1.72 (m, 2H) 1.74-1.85 (m, 4H) 1.95-2.05 (m, 2H) 2.10-2.21 (m, 2H) 2.52-2.60 (m, 2H) 2.62-2.69 (m, 2H) 2.76 (s, 3H) 2.84-2.96 (m, 1H) 3.07-3.14 (m, 2H) 3.15-3.27 (m, 3H) 3.33-3.43 (m, 2H) 3.54-3.65 (m, 2H); HRMS (ESI-TOF) m/z calcd for C₁₈H₃₄N₃O₃S 372.23154 [M+H]⁺, found 372.23151.

### Example 53

### (4-isopropyl-1,4-diazepan-1-yl)(7-(2-methylisonicotinoyl)-7-azaspiro[3.5]nonan-2-yl)methanone

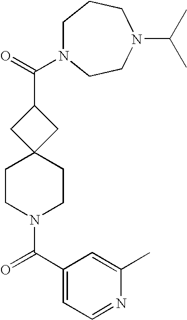

HBTU (233 mg, 0.61 mmol) and Intermediate 13 (120 mg, 0.41 mmol) were added to a solution of 2-methylisonicotinic acid (84 mg, 0.61 mmol) and DIEA (0.143 mL, 0.82 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (95 mg, 56.0%) as a solid. ¹H NMR (400 MHz, CDCl₃) δ ppm 0.99 (dd, J=6.64, 1.56 Hz, 6H) 1.53 (br. s., 1H) 1.59 (br. s., 1H) 1.68 (br. s., 2H) 1.72-1.86 (m, 3H) 1.97-2.12 (m, 2H) 2.12-2.28 (m, 2H) 2.51-2.61 (m, 4H) 2.61-2.70 (m, 2H) 2.90 (dt, J=12.99, 6.59 Hz, 1H) 3.15-3.24 (m, 2H) 3.24-3.32 (m, 1H) 3.33-3.45 (m, 2H) 3.55-3.67 (m, 3H) 3.67-3.76 (m, 1H) 6.99-7.08 (m, 1H) 7.10-7.16 (m, 1H) 8.55 (d, J=5.08 Hz, 1H); HRMS (ESI-TOF) m/z calcd for C₂₄H₃₇N₄O₂ 413.29110 [M+H]⁺, found 413.29118.

### Example 54

### (7-isonicotinoyl-7-azaspiro[3.5]nonan-2-yl)(4-isopropyl-1,4-diazepan-1-yl)methanone

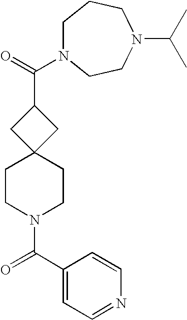

HBTU (233 mg, 0.61 mmol) and Intermediate 13 (120 mg, 0.41 mmol) were added to a solution of isonicotinic acid (76 mg, 0.61 mmol) and DIEA (0.143 mL, 0.82 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (90 mg, 55.3%) as a solid. ¹H NMR (400 MHz, CDCl₃) δ ppm 0.99 (dd, J=6.64, 1.56 Hz, 6H) 1.49-1.57 (m, 1H) 1.57-1.63 (m, 1H) 1.68 (br. s., 2H) 1.72-1.86 (m, 3H) 1.96-2.12 (m, 2H) 2.13-2.28 (m, 2H) 2.57 (q, J=5.34 Hz, 2H) 2.62-2.70 (m, 2H) 2.90 (ddd, J=13.09, 6.84, 6.64 Hz, 1H) 3.16-3.24 (m, 1H) 3.24-3.33 (m, 1H) 3.34-3.46 (m, 2H) 3.55-3.63 (m, 2H) 3.63-3.78 (m, 2H) 7.20-7.31 (m, 2H) 8.68 (d, J=4.69 Hz, 2H); HRMS (ESI-TOF) m/z calcd for C₂₃H₃₅N₄O₂ 399.27545 [M+H]⁺, found 399.27539.

### Example 55

### (4-isopropyl-1,4-diazepan-1-yl)(7-nicotinoyl-7-azaspiro[3.5]nonan-2-yl)methanone

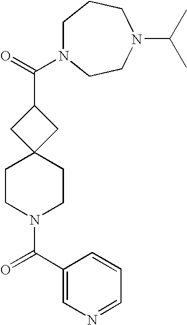

HBTU (233 mg, 0.61 mmol) and Intermediate 13 (120 mg, 0.41 mmol) were added to a solution of nicotinic acid (76 mg, 0.61 mmol) and DIEA (0.143 mL, 0.82 mmol) in DMF (10 mL). The reaction mixture was stirred for 18 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (94 mg, 57.6%) as a solid. ¹H NMR (400 MHz, CDCl₃) δ ppm 0.99 (dd, J=6.64, 1.56 Hz, 6H) 1.56 (br. s., 1H) 1.63 (br. s., 1H) 1.70 (br. s., 1H) 1.74-1.86 (m, 3H) 1.98-2.13 (m, 2H) 2.13-2.29 (m, 2H) 2.52-2.58 (m, 2H) 2.62-2.72 (m, 2H) 2.90 (quintet, J=6.54 Hz, 1H) 3.28 (br. s., 2H) 3.32-3.46 (m, 3H) 3.60 (t, J=6.05 Hz, 2H) 3.63-3.70 (m, 1H) 3.73 (br. s., 1H) 7.36 (dd, J=7.81, 5.08 Hz, 1H) 7.67-7.78 (m, 1H) 8.60-8.69 (m, 2H); HRMS (ESI-TOF) m/z calcd for C₂₃H₃₅N₄O₂ 399.27545 [M+H]⁺, found 399.27529.

### Example 56

### 2-(4-isopropyl-1,4-diazepane-1-carbonyl)-N,N-dimethyl-7-azaspiro[3.5]nonane-7-carboxamide

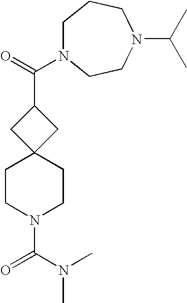

Dimethylcarbamic chloride (52.8 mg, 0.49 mmol) was added to a solution of Intermediate 13 (120 mg, 0.41 mmol) and Et₃N (0.085 mL, 0.61 mmol) in DCM (12 mL) at 0° C. The reaction mixture was stirred for 18 h and the solvent was concentrated. The product was purified on preparative reverse-phase HPLC using a low pH shallow gradient method (Mobile phase: 30-50% B; A: H₂O with 0.05% TFA, B: CH₃CN, 25 min. run) on Luna 15 μm, C18, 21.2×250 mm Phenomenex reverse phase column to provide title compound (107 mg, 71.6%) as a solid. ¹H NMR (400 MHz, CDCl₃) δ ppm 0.99 (d, J=6.25 Hz, 6H) 1.51-1.60 (m, 2H) 1.61-1.69 (m, 2H) 1.73-1.86 (m, 2H) 1.94-2.06 (m, 2H) 2.09-2.19 (m, 2H) 2.52-2.61 (m, 2H) 2.62-2.69 (m, 2H) 2.80 (s, 6H) 2.84-2.95 (m, 1H) 3.04-3.11 (m, 2H) 3.13-3.25 (m, 3H) 3.34-3.44 (m, 2H) 3.55-3.64 (m, 2H); HRMS (ESI-TOF) m/z calcd for C₂₀H₃₇N₄O₂ 365.29110 [M+H]⁺, found 365.29097.

### Example 57

### (4-cyclobutyl-6,6-dimethyl-1,4-diazepan-1-yl)(7-(2-methylisonicotinoyl)-7-azaspiro[3.5]nonan-2-yl)methanone

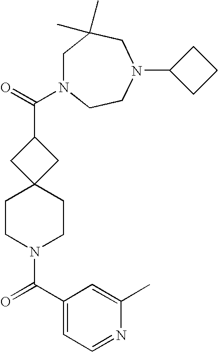

HBTU (200 mg, 0.53 mmol) and Intermediate 16 (100 mg, 0.30 mmol) were added to a solution of 2-methylisonicotinic acid (72.4 mg, 0.53 mmol) and DIEA (0.108 mL, 0.62 mmol) in DMF (15 mL). The reaction mixture was stirred for 4 h and the solvent was concentrated. The product was purified on preparative reverse-phase HPLC using a high pH shallow gradient method (Mobile phase: 20-40% B; A: H₂O with 15 mM NH₄CO₃ and 0.375% NH₄OH v/v, B: CH₃CN, 25 min. run) on XBridge Prep C18 OBD, 30×150 mm, 5 μm, Waters reverse phase column to provide title compound (57.0 mg, 42.0%) as a solid. ¹H NMR (400 MHz, CDCl₃) δ ppm 0.92 (br. s., 6H) 1.50-1.62 (m, 3H) 1.62-1.84 (m, 6H) 1.91-2.12 (m, 6H) 2.12-2.28 (m, 2H) 2.45 (br. s., 2H) 2.59 (s, 3H) 2.89 (br. s., 1H) 3.08-3.23 (m, 2H) 3.24-3.30 (m, 1H) 3.30-3.49 (m, 3H) 3.51-3.67 (m, 1H) 3.71 (br. s., 1H) 6.98-7.09 (m, 1H) 7.12 (d, J=5.47 Hz, 1H) 8.55 (d, J=5.08 Hz, 1H); HRMS (ESI-TOF) m/z calcd for C₂₇H₄₁N₄O₂ 453.32240 [M+H]⁺, found 453.32244.

### Example 58

### (4-cyclopentylpiperazin-1-yl)(7-(methylsulfonyl)-7-azaspiro[3.5]nonan-2-yl)methanone

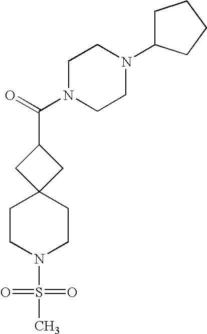

Oxalyl chloride (85 μl, 0.97 mmol) was slowly added to a solution of Intermediate 26 in DCM (12 mL) at 0° C. One drop of DMF was added and the reaction mixture was stirred for 4 h. The solvent was concentrated. The residue was quickly recovered in DCM (5 mL) and added to a solution of 1-cyclopentylpiperazine (54.9 mg, 0.36 mmol) and Et₃N (0.135 mL, 0.97 mmol) in DCM (12 mL) at 0° C. The reaction mixture was allowed to warm to ambiant temperature and stirred for 1 h. The solvent was concentrated and the product was purified on silica gel (24 g) by MPLC using MeOH 5%, acetone 10% in DCM as eluent to provide title compound (106 mg, 85%) as a solid. ¹H NMR (400 MHz, CHLOROFORM-d) δ ppm 1.32-1.47 (m, 2H) 1.49-1.62 (m, 2H) 1.64-1.73 (m, 4H) 1.73-1.80 (m, 2H) 1.80-1.92 (m, 2H) 1.97-2.07 (m, 2H) 2.10-2.20 (m, 2H) 2.40-2.55 (m, 5H) 2.76 (s, 3H) 3.07-3.14 (m, 2H) 3.15-3.26 (m, 3H) 3.32-3.39 (m, 2H) 3.59-3.67 (m, 2H); HRMS m/z calcd for C₁₉H₃₄N₃O₃S 384.2315 [M+H]⁺, found 384.2305.

### Example 59

### ((S)-4-cyclobutyl-3-methylpiperazin-1-yl)(7-(methylsulfonyl)-7-azaspiro[3.5]nonan-2-yl)methanone

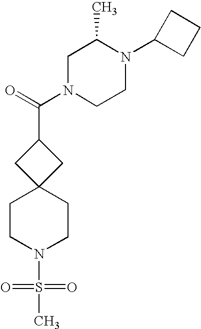

Oxalyl chloride (0.085 mL, 0.97 mmol) was slowly added to a solution of Intermediate 26 (80 mg, 0.32 mmol) in DCM (12 mL) at 0° C. One drop of DMF was added and the reaction mixture was stirred for 4 h. The solvent was concentrated. The residue was quickly recovered in DCM (5 mL) and added to a solution of Intermediate 32 (81 mg, 0.36 mmol) and Et₃N (0.225 mL, 1.62 mmol) in DCM (12 mL) at 0° C. The reaction mixture was allowed to warm to ambiant temperature and stirred for 1 h. The solvent was concentrated and the product was purified on silica gel (24 g) by MPLC using 5% MeOH and 10% acetone in DCM as the eluent to provide title compound (111 mg, 89%) as a solid. ¹H NMR (400 MHz, CHLOROFORM-d) δ ppm 0.98 (d, J=6.64 Hz, 3H) 1.59-1.72 (m, 4H) 1.72-1.80 (m, 2H) 1.87 (dt, J=19.53, 9.77 Hz, 1H) 1.93-2.06 (m, 4H) 2.06-2.22 (m, 4H) 2.48 (td, J=6.64, 3.12 Hz, 1H) 2.57-2.68 (m, 1H) 2.76 (s, 3H) 2.94-3.08 (m, 2H) 3.08-3.15 (m, 2H) 3.14-3.23 (m, 3H) 3.23-3.34 (m, 1H) 3.34-3.57 (m, 1H) 3.66-3.77 (m, 1H); HRMS m/z calcd for C₁₉H₃₄N₃O₃S 384.2315 [M+H]⁺, found 384.2302; [a]_{D}²⁰-−0.1° (c 0.92, MeOH).

### Example 60

### ((R)-4-isopropyl-3-methylpiperazin-1-yl)(7-(methylsulfonyl)-7-azaspiro[3.5]nonan-2-yl)methanone

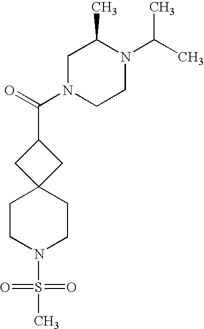

Oxalyl chloride (0.067 mL, 0.76 mmol) was slowly added to a solution of Intermediate 26 (62.9 mg, 0.25 mmol) in DCM (12 mL) at 0° C. One drop of DMF was added and the reaction mixture was stirred for 4 h. The solvent was concentrated. The residue was quickly recovered in DCM (5 mL) and added to a solution of (R)-1-isopropyl-2-methylpiperazine hydrochloride (50 mg, 0.28 mmol) and Et₃N (0.177 mL, 1.27 mmol) in DCM (12 mL) at 0° C. The reaction mixture was allowed to warm to ambiant temperature and stirred for 1 h. The solvent was concentrated and the product was purified on silica gel (24 g) by MPLC using 5% MeOH and 10% acetone in DCM as the eluent to provide title compound (79 mg, 84%) as a solid. ¹H NMR (400 MHz, CHLOROFORM-d) δ ppm 0.87 (d, 3H) 1.05 (dd, J=6.25, 2.73 Hz, 3H) 1.11 (dd, J=6.64, 2.73 Hz, 3H) 1.68 (t, J=5.47 Hz, 2H) 1.76 (td, J=5.37, 3.32 Hz, 2H) 1.96-2.07 (m, 2H) 2.10-2.18 (m, 2H) 2.18-2.27 (m, 1H) 2.45-2.57 (m, J=8.94, 8.94, 6.15, 2.93 Hz, 1H) 2.66 (dd, J=12.89, 9.77 Hz, 1H) 2.70-2.75 (m, 1H) 2.76 (s, 3H) 2.80-3.00 (m, 1H) 3.06-3.14 (m, 2H) 3.14-3.28 (m, 4H) 3.34-3.55 (m, 1H) 4.13-4.27 (m, 1H); HRMS m/z calcd for C₁₈H₃₄N₃O₃S 372.2315 [M+H]⁺, found 372.23; [a]_{D}²⁰-−5.8° (c 0.31, MeOH).

### Example 61

### ((S)-4-isopropyl-3-methylpiperazin-1-yl)(7-(methylsulfonyl)-7-azaspiro[3.5]nonan-2-yl)methanone

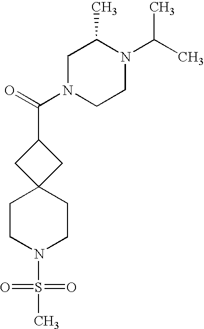

Oxalyl chloride (0.085 mL, 0.97 mmol) was slowly added to a solution of Intermediate 26 in DCM (12 mL) at 0° C. One drop of DMF was added and the reaction mixture was stirred for 4 h. The solvent was concentrated. The residue was quickly recovered in DCM (5 mL) and added to a solution of (S)-1-isopropyl-2-methylpiperazine dihydrochloride (77 mg, 0.36 mmol) and Et₃N (0.225 mL, 1.62 mmol) in DCM (12 mL) at 0° C. The reaction mixture was allowed to warm to ambiant temperature and stirred for 1 h. The solvent was concentrated and the product was purified on silica gel (24 g) by MPLC using 5% MeOH and 10% acetone in DCM as the eluent to provide title compound (111 mg, 92%) as a solid. ¹H NMR (400 MHz, CHLOROFORM-d) δ ppm 0.87 (d, J=6.25 Hz, 3H) 1.05 (dd, J=6.25, 2.73 Hz, 3H) 1.11 (dd, J=6.64, 2.73 Hz, 3H) 1.68 (t, J=5.47 Hz, 2H) 1.73-1.80 (m, 2H) 1.96-2.08 (m, 2H) 2.10-2.19 (m, 2H) 2.19-2.27 (m, 1H) 2.46-2.57 (m, J=9.08, 9.08, 6.25, 2.93 Hz, 1H) 2.62-2.72 (m, 1H) 2.74 (t, J=3.12 Hz, 1H) 2.76 (s, 3H) 2.80-3.00 (m, 1H) 3.06-3.15 (m, 2H) 3.15-3.29 (m, 4H) 3.34-3.54 (m, 1H) 4.13-4.28 (m, 1H); HRMS m/z calcd for C₁₈H₃₄N₃O₃S 372.2315 [M+H]⁺, found 372.231; [a]_{D}²⁰+10.1° (c 0.52, MeOH).

### Example 62

### ((R)-4-cyclobutyl-2-methylpiperazin-1-yl)(7-(methylsulfonyl)-7-azaspiro[3.5]nonan-2-yl)methanone

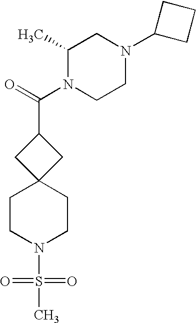

A mixture of Intermediate 28 (105 mg, 0.24 mmol), Pd/C (2.52 mg, 0.02 mmol), cyclobutanone (332 mg, 4.74 mmol) and ethanol was shaken for 18 h in a Parr hydrogenation apparatus under a 50 psi hydrogen atmosphere. The mixture was filtered over a celite pad and the filtrate was concentrated. The product was purified by preparative HPLC UV using a high pH shallow gradient method (Mobile phase: 20-40% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to afford title compound (65.8 mg, 72.5%) as a solid. ¹H NMR (400 MHz, CHLOROFORM-d) δ ppm 1.13-1.38 (m, 3H) 1.55-1.73 (m, 5H) 1.73-1.80 (m, 3H) 1.80-1.92 (m, 2H) 1.92-2.05 (m, 4H) 2.05-2.28 (m, 2H) 2.53-2.68 (m, 2H) 2.70-2.98 (m, 5H) 3.11 (d, J=5.08 Hz, 2H) 3.14-3.30 (m, 3H) 3.31-3.87 (m, 1H) 4.26-4.78 (m, 1H); HRMS m/z calcd for C₁₉H₃₄N₃O₃S 384.2315 [M+H]⁺, found 384.2315; [α]_{D}²³−37.9 (c 1.22, MeOH).

### Example 63

### ((S)-4-cyclobutyl-2-methylpiperazin-1-yl)(7-(methylsulfonyl)-7-azaspiro[3.5]nonan-2-yl)methanone

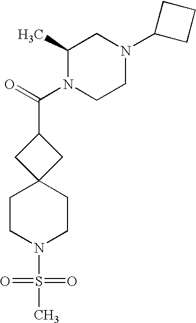

A mixture of Intermediate 30 (105 mg, 0.24 mmol), Pd/C (2.52 mg, 0.02 mmol), cyclobutanone (332 mg, 4.74 mmol) and ethanol was shaken for 18 h in a Parr hydrogenation apparatus under a 50 psi hydrogen atmosphere. The mixture was filtered over a celite pad and the filtrate was concentrated. The product was purified by preparative HPLC UV using a high pH shallow gradient method (Mobile phase: 20-40% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to afford title product (57.5 mg, 63.3%) as a solid. ¹H NMR (400 MHz, CHLOROFORM-d) d ppm 1.15-1.37 (m, 3H) 1.57-1.73 (m, 6H) 1.73-1.81 (m, 3H) 1.81-1.92 (m, 2H) 1.92-2.05 (m, 4H) 2.05-2.30 (m, 2H) 2.54-2.69 (m, 2H) 2.69-2.99 (m, 4H) 3.11 (d, J=4.30 Hz, 2H) 3.14-3.30 (m, 3H) 3.30-3.87 (m, 1H) 4.27-4.79 (m, 1H); HRMS m/z calcd for C₁₉H₃₄N₃O₃S 384.2315 [M+H]⁺, found 384.2308; [a]_{D}²³+37.1 (c 1.30, MeOH).

### Example 64

### 4-(2-(4-cyclobutylpiperazine-1-carbonyl)-7-azaspiro[3.5]nonane-7-carbonyl)nicotinonitrile

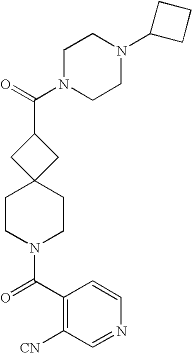

HBTU (234 mg, 0.62 mmol) and Intermediate 7 (120 mg, 0.41 mmol) were added to a solution of Intermediate 35 (91 mg, 0.62 mmol) and DIEA (0.216 mL, 1.24 mmol) in DMF (12 mL). The reaction mixture was stirred for 3 h and the solvent was concentrated. The product was purified by preparative HPLC UV using a high pH shallow gradient method (Mobile phase: 20-40% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to provide title compound (86 mg, 49.5%) as a solid. ¹H NMR (400 MHz, CHLOROFORM-d) d ppm 1.53-1.64 (m, 1H) 1.64-1.76 (m, 4H) 1.76-1.94 (m, 3H) 1.97-2.10 (m, 4H) 2.10-2.23 (m, 2H) 2.23-2.37 (m, 4H) 2.71 (quin, J=7.71 Hz, 1H) 3.10-3.17 (m, 1H) 3.17-3.30 (m, 2H) 3.30-3.43 (m, 2H) 3.56-3.67 (m, 2H) 3.67-3.74 (m, 1H) 3.74-3.83 (m, 1H) 7.37 (dd, J=8.01, 5.27 Hz, 1H) 8.86 (dd, J=5.08, 2.73 Hz, 1H) 8.94 (s, 1H); HRMS m/z calcd for C₂₄H₃₂N₅O₂ 422.2551[M+H]⁺, found 422.254.

### Example 65

### (4-cyclobutylpiperazin-1-yl)(7-(pyrazin-2-yl)-7-azaspiro[3.5]nonan-2-yl)methanone

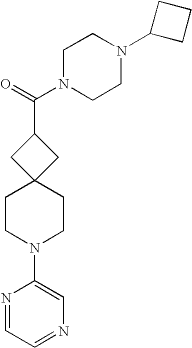

A mixture of Intermediate 7 (100 mg, 0.34 mmol), 2-fluoropyrazine (141 mg, 1.44 mmol), DIEA (0.6 mL, 3.44 mmol) and DMSO (1 mL) was heated to 130° C. for 30 min in a Biotage Initiator microwave oven. The reaction mixture was injected on a preparative HPLC UV using a high pH shallow gradient method (Mobile phase: 30-50% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to provide title compound (76 mg, 59.9%) as a solid. ¹H NMR (400 MHz, CHLOROFORM-d) δ ppm 1.59-1.68 (m, 2H) 1.68-1.82 (m, 4H) 1.91 (br. s., 2H) 1.99-2.12 (m, 4H) 2.13-2.24 (m, 2H) 2.30 (br. s., 4H) 2.74 (quin, J=7.52 Hz, 1H) 3.22 (quin, J=8.69 Hz, 1H) 3.39 (br. s., 2H) 3.45-3.53 (m, 2H) 3.54-3.62 (m, 2H) 3.65 (br. s., 2H) 7.79 (d, J=2.34 Hz, 1H) 8.03 (dd, J=2.73, 1.56 Hz, 1H) 8.14 (s, 1H); HRMS m/z calcd for C₂₁H₃₂N₅O 370.2601 [M+H]⁺, found 370.2604.

### Example 66

### (4-cyclobutylpiperazin-1-yl)(7-(pyridin-4-yl)-7-azaspiro[3.5]nonan-2-yl)methanone

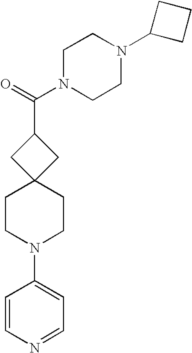

To a mixture of PdOAc₂ (7.70 mg, 0.03 mmol) and BINAP (42.7 mg, 0.07 mmol) in toluene (3 mL) was added respectively Intermediate 7 (100 mg, 0.34 mmol), 4-bromopyridine hydrochloride (70.1 mg, 0.36 mmol) and CS₂CO₃ (235 mg, 0.72 mmol). The mixture was purged with nitrogen and heated to 140° C. for 45 min using a Biotage Initiator microwave oven. The reaction mixture was filtered and the solvent was concentrated. The product was purified by preparative HPLC UV using a high pH shallow gradient method (Mobile phase: 30-50% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to provide title product (30.0 mg, 23.72%) as a solid. ¹H NMR (400 MHz, CHLOROFORM-d) δ ppm 1.66 (dd, J=7.03, 4.69 Hz, 2H) 1.69-1.79 (m, 3H) 1.79-1.95 (m, 2H) 1.96-2.12 (m, 4H) 2.12-2.23 (m, 2H) 2.28 (ddd, J=7.62, 5.27, 5.08 Hz, 4H) 2.44 (br. s., 1H) 2.71 (quin, J=7.91 Hz, 1H) 3.15-3.25 (m, 1H) 3.25-3.31 (m, 2H) 3.32-3.43 (m, 4H) 3.57-3.71 (m, 2H) 6.68 (d, J=3.52 Hz, 2H) 8.22 (br. s., 2H); HRMS m/z calcd for C₂₂H₃₃N₄O 369.2649 [M+H]⁺, found 369.2642.

### Example 67

### (4-cyclobutylpiperazin-1-yl)(7-(pyrimidin-5-yl)-7-azaspiro[3.5]nonan-2-yl)methanone

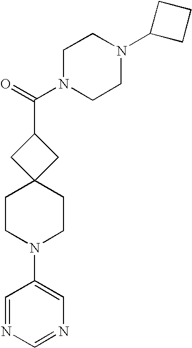

To a mixture of PdOAc₂ (7.70 mg, 0.03 mmol) and BINAP (42.7 mg, 0.07 mmol) in toluene (3 mL) was added respectively Intermediate 7 (100 mg, 0.34 mmol), 5-bromopyrimidine (57.3 mg, 0.36 mmol) and Cs₂CO₃ (123 mg, 0.38 mmol). The mixture was purged with nitrogen and heated to 140° C. for 45 min using a Biotage Initiator microwave oven. The reaction mixture was filtered and the solvent was concentrated. The product was purified by preparative HPLC UV using a high pH shallow gradient method (Mobile phase: 30-50% B; A: H₂O with 15 mM NH₄CO₃ and 0.375% NH₄OH v/v, B: CH₃CN, 30 min. run) on XBridge Prep C18 OBD, 30×150 mm, 10 μm, Waters reverse phase column, to provide title compound (23.00 mg, 18.14%) as a solid. ¹H NMR (400 MHz, CHLOROFORM-d) δ ppm 1.62-1.76 (m, 4H) 1.76-1.83 (m, 2H) 1.83-1.94 (m, 2H) 1.97-2.12 (m, 4H) 2.13-2.23 (m, 2H) 2.23-2.35 (m, 4H) 2.71 (quin, J=7.81 Hz, 1H) 3.10-3.18 (m, 2H) 3.18-3.28 (m, 3H) 3.31-3.41 (m, 2H) 3.58-3.69 (m, 2H) 8.36 (s, 2H) 8.65 (s, 1H); HRMS m/z calcd for C₂₁H₃₂N₅O 370.2601 [M+H]⁺, found 370.2595.

### Intermediate 1

### Benzyl 4-methylenepiperidine-1-carboxylate

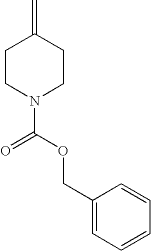

To a flame-dried three-necked round bottom flask equipped with a thermometer, N₂ bubbler and an addition funnel was charged (Ph)₃PCH₃Br (39 g, 0.109 mol) and 280 mL of anhydrous THF. The resultant suspension was stirred under N₂ while being cooled to 0° C. To this suspension was added, dropwise, n-BuLi (68 mL, 0.109 mol) at 0° C. over a period of 1 h. After the addition was finished, the yellowish orange suspension formed was stirred at 0° C. for another hour. A solution of benzyl 4-oxopiperidine-1-carboxylate (20.0 g, 0.0836 mol) in 160 mL of anhydrous THF was charged to this suspension at 0° C. over a period of 1 h. After completion of the addition, the resultant mixture was stirred at room temperature for a period of 3 h. The reaction was quenched with 200 mL of H₂O and extracted twice with 300 mL of EtOAc. The combined organic layers were washed with brine, dried over anhydrous MgSO₄, filtered and evaporated to dryness to give 45 g of the crude product as a yellow solid. This crude material was purified by passing through a short column of silica gel, using hexane/EtOAc 4:1 as the mobile phase, to provide title compound (17.70 g, 92%) as an oil. ¹H NMR (400 MHz, CDCl₃) δ ppm 1.22 (m, 4H) 3.53 (m, 4H) 4.78 (s, 2H) 5.17 (s, 2H) 7.30-7.44 (m, 5H).

### Intermediate 2

### benzyl 2-oxo-7-azaspiro[3.5]nonane-7-carboxylate

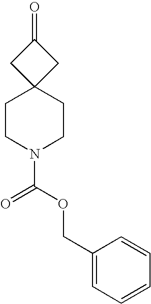

Activation of zinc: A stirred suspension of 200 g of zinc dust in H₂O was degassed by bubbling N₂ through it for 0.5 h. Copper (II) sulphate (5.5 g) was charged and the resultant suspension was stirred at room temperature for 1 h. The Zn—Cu couple produced was collected on a glass funnel under a stream of N₂ and washed with degassed H₂O and acetone. The Zn—Cu couple was dried in a vacuum oven at 40° C. overnight. A 5 L round bottomed flask was charged with Intermediate 1 (62.5 g, 0.2702 mol) and MTBE (1100 mL). The solution formed was stirred at room temperature. To this solution was added 200 g of the freshly prepared Zn—Cu couple and the resultant suspension was cooled to 10-15° C. with stirring. To this mixture was slowly added a solution of Cl₃COCl (106 mL, 0.9357 mol) in 438 mL of DME over a period of 1 h. An exotherm from 15 to 30° C. was observed during the addition. After the addition was finished, the reaction mixture was stirred at room temperature overnight. The reaction mixture was cooled to 0-5° C. and 1000 mL of saturated NH₄Cl solution was slowly charged to the reaction flask while maintaining an internal temperature between 15 and 30° C. After the addition was completed, the mixture was stirred at room temperature for 4 h. The reaction mixture was filtered and the filtrate was extracted twice with 600 mL of EtOAc. The combined organic layers was washed with sat. NH₄Cl solution, dried over anhydrous MgSO₄, filtered and evaporated to dryness to give 75 g of the crude cyclobutanone. The crude product was purified by column chromatography (silica, hexanes/EtOAc 7:3) to afford title compound (61 g, 83%) as an oil. ¹H NMR (400 MHz, CDCl₃) δ ppm 1.71 (m, 4H) 2.90 (s, 4H) 3.48 (m, 4H) 5.13 (s, 2H) 7.28-7.36 (m, 5H)

### Intermediate 3

### benzyl 2-(methoxymethylene)-7-azaspiro[3.5]nonane-7-carboxylate

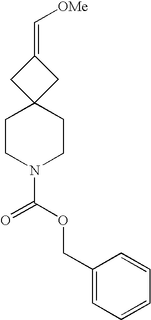

To a flame-dried three-necked round bottom flask equipped with a thermometer, N₂ bubbler and an addition funnel was charged a solution of KO^{t}Bu in THF (625 mL, 0.6256 mol), ^{t}BuOH (46.4 g, 0.6256 mol) and anhydrous THF (500 mL). The resultant mixture was stirred under N₂ while being cooled to −78° C. The Wittig reagent (Ph)₃PCH₂(OMe)Cl (214.5 g, 0.6256 mol) was charged portion-wise to the flask. The resultant mixture was allowed to warm up to 0° C. and stirred at this temperature for another hour. The orange solution produced was cooled to −78° C. and a solution of Intermediate 2 (57 g, 0.2085 mol) in anhydrous THF (500 mL) was added dropwise to the reaction mixture over a period of 0.5 h. After the addition was finished, the reaction mixture was allowed to warm up to 0° C. and stirred at this temperature for 1 h. The reaction was quenched with H₂O (1000 mL) and extracted with twice with 500 mL of EtOAc. The organic layers were combined, washed with brine, dried over anhydrous MgSO₄ and filtered. The solvent was evaporated and the crude was purified by column chromatography (silica, hexanes/EtOAc 4:1) to give title compound (53 g, 84%). ¹H NMR (400 MHz, CDCl₃) δ ppm 1.28 (m, 4H) 2.36 (m, 2H) 2.46 (m, 2H) 3.42 (m, 4H) 3.57 (s, 3H) 5.14 (s, 2H) 5.87 (quintet, J=2 Hz, 1H), 7.27-7.38 (m, 5H).

### Intermediate 4

### benzyl 2-formyl-7-azaspiro[3.5]nonane-7-carboxylate

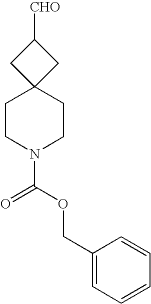

To a 2000 mL round bottomed flask equipped with mechanical stirrer, N₂ bubbler and a thermometer, was charged Intermediate 3 (100 g, 0.3318 mol), CH₃CN (1000 mL) and HCl (500 mL, 2 M). The resultant mixture was stirred at room temperature for 4 h. The reaction mixture was concentrated under vacuum and the residue was extracted twice with 500 mL of EtOAc. The organic layers were combined, washed with brine, dried over MgSO₄, filtered and evaporated to dryness to give title compound (94 g, 99%) as an orange oil, which was used in the next step without purification. ¹H NMR (400 MHz, CDCl₃) δ ppm 1.49 (m, 2H) 1.64 (m, 2H) 1.98-2.12 (m, 4H) 3.15 (quintet, J=8.5 Hz, 1H) 3.37-3.47 (m, 4H) 5.13 (s, 2H) 7.34 (m, 5H) 9.77 (s, 1H).

### Intermediate 5

### 7-(benzyloxycarbonyl)-7-azaspiro[3.5]nonane-2-carboxylic acid

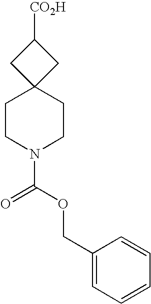

To a 3000 mL round bottomed flask equipped with mechanical stirrer, addition funnel, a N₂ bubbler and a thermometer, was charged Intermediate 4 (92 g, 0.3202 mol), H₂O (740 mL), saturated NaHCO₃ solution (153 g, 1.8250 mol), NaBr (6.6 g, 0.0641 mol), and a solution of TEMPO in DCM (156 mL, 1 mg of TEMPO per 1 mL). With agitation, commercial bleach solution (920 mL) was slowly added to this mixture over a period of 2 h. After the addition was finished, the mixture was stirred at ambient temperature for 3 h. The reaction mixture was washed with EtOAc (2×500 mL) and its pH was adjusted to 1-2 by the addition of concentrated HCl. The resultant mixture was extracted with 2×500 mL of EtOAc and 2×500 mL of MTBE. The combined organic layers were dried over anhydrous MgSO₄, filtered and evaporated to dryness to give 58 g of a light yellow oil. This crude product was purified by column chromatography (silica, hexane/EtOAc 3:2) to give title compound (42 g, 43%). ¹H NMR analysis showed there was a small amount of impurities present in this material. ¹H NMR (400 MHz, CDCl₃) δ 1 ppm 1.56-1.61 (m, 4H) 2.10 (s, 2H) 2.12 (s, 2H) 3.13 (quintet, J=8.5 Hz, 1H) 3.37-3.46 (m, 4H) 5.13 (s, 2H) 7.28-7.39 (m, 5H).

### Intermediate 6

### benzyl 2-(4-cyclobutylpiperazine-1-carbonyl)-7-azaspiro[3.5]nonane-7-carboxylate

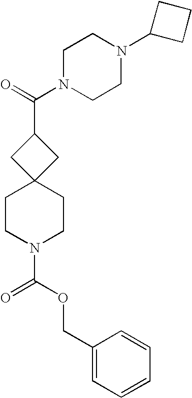

HBTU (1320 mg, 3.48 mmol) and Intermediate 18 (444 mg, 3.16 mmol) were added to a solution of Intermediate 5 (960 mg, 3.16 mmol) and DIEA (0.663 mL, 3.80 mmol) in DMF (60 mL). The reaction mixture was stirred for 3 h and the solvent was concentrated. The product was purified on silica gel by MPLC using 3% MeOH and 5% acetone in DCM with 0.1N ammonia (80 g column; 20 mL/min then 40 mL/min) to provide title compound (1300 mg, 97%) as a solid. ¹H NMR (400 MHz, CDCl₃) δ ppm 1.43-1.57 (m, 3H) 1.56-1.67 (m, 2H) 1.67-1.82 (m, 2H) 1.82-1.98 (m, 3H) 1.98-2.08 (m, 3H) 2.08-2.18 (m, 2H) 2.26-2.35 (m, 3H) 2.69-2.80 (m, 1H) 3.18 (quintet, J=8.50 Hz, 1H) 3.33-3.42 (m, 4H) 3.41-3.50 (m, 2H) 3.58-3.69 (m, 2H) 5.12 (s, 2H) 7.29-7.41 (m, 5H); MS m/z 426.4 [M+H]⁺ (ESI).

### Intermediate 7

### (4-cyclobutylpiperazin-1-yl)(7-azaspiro[3.5]nonan-2-yl)methanone

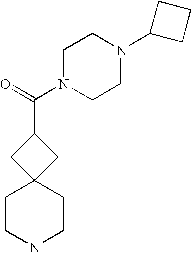

A mixture of Intermediate 6 (1.29 g, 3.03 mmol), Pd/C (0.016 g, 0.15 mmol) and ethanol (200 mL) was shaken in a Parr apparatus under a 50 psi atmosphere of H₂ for 4.5 h. The mixture was filtered over a celite bed and the solvent was concentrated to provide title compound (0.880 g, 100%) as a solid. ¹H NMR (400 MHz, CDCl₃) δ ppm 1.54-1.66 (m, 2H) 1.66-1.79 (m, 4H) 1.80-1.95 (m, 2H) 1.95-2.08 (m, 4H) 2.08-2.17 (m, 2H) 2.27 (q, J=5.21 Hz, 4H) 2.71 (ddd, J=15.53, 8.20, 7.91 Hz, 1H) 2.78-2.86 (m, 2H) 2.86-2.94 (m, 2H) 3.07 (br. s., 1H) 3.12-3.24 (m, 1H) 3.29-3.41 (m, 2H) 3.56-3.68 (m, 2H); MS m/z 292.2 [M+H]⁺ (ESI).

### Intermediate 8

### benzyl 2-(4-isopropylpiperazine-1-carbonyl)-7-azaspiro[3.5]nonane-7-carboxylate

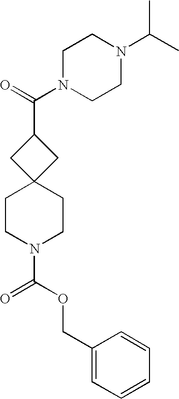

Oxalyl chloride (2.39 mL, 27.3 mmol) was slowly added to a solution of Intermediate 5 (2.77 g, 9.13 mmol) in DCM (70 mL) at 0° C. under N₂. One drop of DMF was added and the reaction mixture was stirred for 2 h. Oxalyl chloride (1.59 mL, 18.2 mmol) was added and the reaction mixture was stirred for 1 more hour. The solvent was concentrated under vacuum and the residue was recovered in DCM (30 mL). The resulting solution was added to a solution of 1-isopropylpiperazine (1.171 g, 9.13 mmol) and Et₃N (6.36 mL, 45.66 mmol) in DCM (150 mL) at 0° C. The reaction mixture was stirred for 1 h. The solvent was concentrated and the product was purified on silica gel by MPLC using 3%-5% MeOH in DCM as eluent to provide title compound (1.810 g, 47.9%) as a gum. ¹H NMR (400 MHz, CDCl₃) δ ppm 1.03 (d, J=6.25 Hz, 6H) 1.52 (br. s., 2H) 1.61 (br. s., 2H) 1.94-2.07 (m, 2H) 2.08-2.18 (m, 2H) 2.39-2.52 (m, 4H) 2.70 (quintet, J=6.54 Hz, 1H) 3.18 (qd, J=8.66, 8.40 Hz, 1H) 3.29-3.40 (m, 4H) 3.40-3.49 (m, 2H) 3.57-3.65 (m, 2H) 5.11 (s, 2H) 7.28-7.42 (m, 5H); MS m/z 414.3 [M+H]⁺ (ESI).

### Intermediate 9

### (4-isopropylpiperazin-1-yl)(7-azaspiro[3.5]nonan-2-yl)methanone

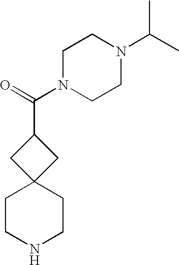

A mixture of Intermediate 8 (1.80 g, 4.35 mmol), Pd/C (0.023 g, 0.22 mmol) and ethanol (100 mL) was shaken in a Parr apparatus under a 50 psi atmosphere of H₂ for 4 h. The mixture was filtered over a celite pad and the solvent was concentrated to provide title compound (1.200 g, 99%) that was used for the next step without further purification. ¹H NMR (400 MHz, CDCl₃) δ ppm 1.04 (d, J=6.64 Hz, 6H) 1.53-1.64 (m, 2H) 1.64-1.74 (m, 2H) 1.93-2.07 (m, 2H) 2.07-2.19 (m, 2H) 2.46 (q, J=5.21 Hz, 4H) 2.70 (dt, J=12.89, 6.45 Hz, 1H) 2.74-2.81 (m, 2H) 2.81-2.99 (m, 3H) 3.16 (quintet, J=8.69 Hz, 1H) 3.28-3.41 (m, 2H) 3.56-3.67 (m, 2H); MS m/z 280.2 [M+H]⁺ (ESI).

### Intermediate 10

### benzyl 2-(4-cyclobutyl-1,4-diazepane-1-carbonyl)-7-azaspiro[3.5]nonane-7-carboxylate

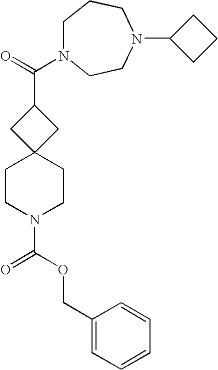

HBTU (2.448 g, 6.45 mmol) and Intermediate 20 (0.905 g, 5.87 mmol) were added to a solution of Intermediate 5 (1.78 g, 5.87 mmol), DIEA (1.230 mL, 7.04 mmol) and DMF (70 mL). The reaction mixture was stirred for 3 h and the solvent was concentrated. Purification by HPLC MS using the short low pH shallow gradient method (mobile phase: 40-60% B; A: H₂O with 0.05% TFA, B: CH₃CN, 10 min. run) on Synergi 4μ Polar-RP 80A, 30×50 mm Phenomenex reverse phase column and extraction with EtOAc provided title compound (0.280 g, 10.86%) as colorless gum. ¹H NMR (400 MHz, CDCl₃) δ ppm 1.45-1.57 (m, 1H) 1.57-1.73 (m, 4H) 1.73-1.93 (m, 5H) 1.94-2.09 (m, 4H) 2.10-2.20 (m, 2H) 2.35-2.44 (m, 2H) 2.44-2.53 (m, 2H) 2.84 (quintet, J=7.81 Hz, 1H) 3.19 (quintet, J=8.69 Hz, 1H) 3.33-3.40 (m, 2H) 3.40-3.50 (m, 4H) 3.57-3.68 (m, 2H) 5.12 (s, 2H) 7.29-7.41 (m, 5H); MS m/z 440.4 [M+H]⁺ (ESI).

### Intermediate 11

### (4-cyclobutyl-1,4-diazepan-1-yl)(7-azaspiro[3.5]nonan-2-yl)methanone

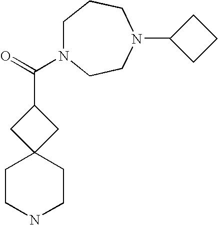

A mixture of Intermediate 10 (270 mg, 0.61 mmol), Pd/C (3.27 mg, 0.03 mmol) and ethanol (70 mL) was shaken in a Parr apparatus under a 50 psi atmosphere of H₂ for 4.5 h. The mixture was filtered over a celite bed and the solvent was concentrated to provide title compound (180 mg, 96%) as a solid used for the next step without further purification. ¹H NMR (400 MHz, CDCl₃) δ ppm 1.49-1.58 (m, 2H) 1.58-1.74 (m, 3H) 1.74-1.93 (m, 4H) 1.93-2.17 (m, 8H) 2.36-2.45 (m, 2H) 2.48 (td, J=4.98, 2.15 Hz, 2H) 2.67-2.78 (m, 2H) 2.78-2.91 (m, 3H) 3.17 (quintet, J=8.79 Hz, 1H) 3.37-3.47 (m, 2H) 3.57-3.67 (m, 2H); MS m/z 306.2 [M+H]⁺ (ESI).

### Intermediate 12

### benzyl 2-(4-isopropyl-1,4-diazepane-1-carbonyl)-7-azaspiro[3.5]nonane-7-carboxylate

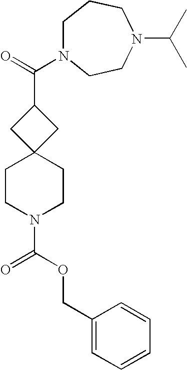

Oxalyl chloride (1.90 mL, 21.7 mmol) was slowly added to a solution of Intermediate 5 (2.20 g, 7.25 mmol) in DCM (70 mL) at 0° C. under N₂. One drop of DMF was added and the reaction mixture was stirred for 2 h. Oxalyl chloride (1.27 mL, 14.5 mmol) was added and the reaction mixture was stirred for 1 more hour. The solvent was concentrated under vacuum and the residue was redissolved in DCM (30 mL). The resulting solution was added to a solution of Intermediate 22 (1.716 g, 7.98 mmol) and Et₃N (5.05 mL, 36.26 mmol) in DCM (150 mL) at 0° C. The reaction mixture was stirred for 1 h. The solvent was concentrated and the product was purified on silica gel by MPLC using 3-5% MeOH in DCM as eluent to provide title compound (2.070 g, 66.8%) as colorless gum. ¹H NMR (400 MHz, CDCl₃) δ ppm 1.00 (dd, J=6.64, 2.73 Hz, 6H) 1.54 (br. s., 2H) 1.62 (br. s., 2H) 1.74-1.89 (m, 2H) 1.94-2.06 (m, 2H) 2.09-2.21 (m, 2H) 2.53-2.62 (m, 2H) 2.66 (ddd, J=10.35, 5.08, 4.88 Hz, 2H) 2.84-2.98 (m, 1H) 3.13-3.26 (m, 1H) 3.33-3.42 (m, 4H) 3.42-3.49 (m, 2H) 3.55-3.66 (m, 2H) 5.11 (s, 2H) 7.29-7.42 (m, 5H); MS m/z 429.4 [M+H]⁺ (ESI).

### Intermediate 13

### (4-isopropyl-1,4-diazepan-1-yl)(7-azaspiro[3.5]nonan-2-yl)methanone

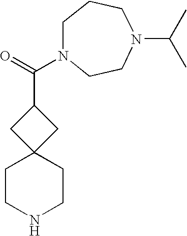

A mixture of Intermediate 12 (2.05 g, 4.79 mmol), Pd/C (0.026 g, 0.24 mmol) and ethanol (120 mL) was shaken in a Parr apparatus under a 50 psi atmosphere of H₂ for 4 h. The mixture was filtered over a celite pad and the solvent was concentrated to provide title compound (1.390 g, 99%) that was used for the next step without further purification. ¹H NMR (400 MHz, CDCl₃) δ ppm 0.99 (dd, J=6.64, 1.95 Hz, 6H) 1.52-1.61 (m, 2H) 1.62-1.70 (m, 2H) 1.73-1.86 (m, 2H) 1.93-2.05 (m, 2H) 2.07-2.17 (m, 2H) 2.49 (br. s., 2H) 2.54-2.61 (m, 2H) 2.61-2.69 (m, 2H) 2.70-2.79 (m, 2H) 2.80-2.86 (m, 1H) 2.90 (dt, J=13.28, 6.64 Hz, 1H) 3.10-3.25 (m, 1H) 3.33-3.44 (m, 2H) 3.54-3.65 (m, 2H); MS m/z 294.2 [M+H]⁺ (ESI).

### Intermediate 14

### benzyl 2-(6,6-dimethyl-1,4-diazepane-1-carbonyl)-7-azaspiro[3.5]nonane-7-carboxylate

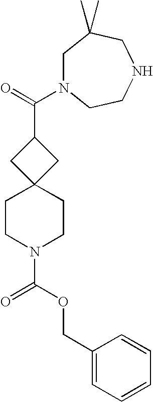

Oxalyl chloride (2.29 mL, 26.2 mmol) was slowly added to a solution of Intermediate 5 (9.25 g, 30.49 mmol) in DCM (200 mL) at 0° C. under N₂. One drop of DMF was added and the reaction mixture was stirred for 2 h. Oxalyl chloride (1.52 mL, 17.4 mmol) was added and the reaction mixture was stirred for 1 more hour. The solvent was concentrated under vacuum and the residue was redissolved in DCM (10 mL). A part of the solution of benzyl 2-(chlorocarbonyl)-7-azaspiro[3.5]nonane-7-carboxylate (2.279 g, 7.08 mmol, 2.32 mL) was added over 1 h via pump syringe to a solution of 6,6-dimethyl-1,4-diazepane (0.908 g, 7.08 mmol) and Et₃N (4.94 mL, 35.41 mmol) in DCM (800 mL) at 0° C. The mixture was stirred for 2 h at 0° C. The solvent was concentrated. The crude material was purified on preparative HPLC using a low pH shallow gradient method (Mobile phase: 30-95% B; A: H₂O with 0.05% TFA, B: CH₃CN, 50 min. run) on Luna 15 μm, C18, 50×250 mm Phenomenex reverse phase column. The volume of the combined purified fraction was reduced and neutralized using sodium bicarbonate. The product was extracted using EtOAc. The organic was dried over anhydrous Na₂SO₄, filtered and concentrated to provide title compound (0.154 g, 5.26%) as a gum. ¹H NMR (400 MHz, CDCl₃) δ ppm 0.92 (s, 6H) 1.55 (br. s., 2H) 1.66 (br. s., 5H) 1.94-2.07 (m, 2H) 2.10-2.21 (m, 2H) 2.91-3.01 (m, 2H) 3.13-3.23 (m, 1H) 3.33-3.40 (m, 3H) 3.42 (s, 1H) 3.43-3.49 (m, 2H) 3.58 (dd, J=6.64, 5.08 Hz, 1H) 5.12 (s, 2H) 7.29-7.40 (m, 5H); MS m/z 414.3 [M+H]⁺ (ESI).

### Intermediate 15

### benzyl 2-(4-cyclobutyl-6,6-dimethyl-1,4-diazepane-1-carbonyl)-7-azaspiro[3.5]nonane-7-carboxylate

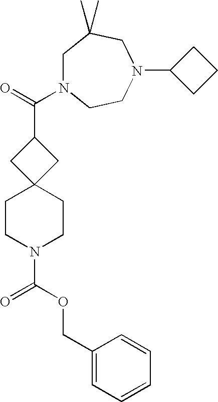

Sodium triacetoxyborohydride (149 mg, 0.70 mmol) was added to a solution of Intermediate 14 (145 mg, 0.35 mmol) and cyclobutanone (36.9 mg, 0.53 mmol) in ethanol (10 mL) at room temperature. The solution was stirred for 30 min and the solvent was concentrated.

The residue was purified by preparative HPLC using a low pH shallow gradient method (Mobile phase: 5-95% B; A: H₂O with 0.05% TFA, B: CH₃CN, 25 min. run) on Luna 15 μm, C18, 21.2×250 mm Phenomenex reverse phase column. The volume of the combined purified fractions was reduced and neutralized using sodium bicarbonate. The product was extracted using EtOAc. The organic was dried over anhydrous Na₂SO₄, filtered and concentrated to provide title compound (70.0 mg, 42.7%) as colorless gum. ¹H NMR (400 MHz, CDCl₃) δ ppm 0.92 (s, 6H) 1.48-1.68 (m, 7H) 1.68-1.83 (m, 2H) 1.93-2.03 (m, 3H) 2.03-2.08 (m, 2H) 2.09-2.19 (m, 2H) 2.40-2.50 (m, 2H) 2.89 (quintet, J=7.62 Hz, 1H) 3.14-3.25 (m, 1H) 3.28-3.41 (m, 5H) 3.41-3.49 (m, 2H) 3.53-3.60 (m, 1H) 5.12 (s, 2H) 7.29-7.40 (m, 5H); MS m/z 468.4 [M+H]⁺ (ESI).

### Intermediate 16

### (4-cyclobutyl-6,6-dimethyl-1,4-diazepan-1-yl)(7-azaspiro[3.5]nonan-2-yl)methanone

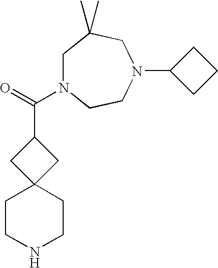

A mixture of Intermediate 15 (230 mg, 0.49 mmol), Pd/C (2.62 mg, 0.02 mmol) and ethanol (30 mL) was shaken in a Parr apparatus under a 50 psi atmosphere of H₂ for 4 h. The mixture was filtered over a celite pad and the solvent was concentrated to provide title compound (160 mg, 98%) that was used for the next step without further purification. ¹H NMR (400 MHz, CDCl₃) δ ppm 0.91 (s, 6H) 1.52-1.69 (m, 5H) 1.69-1.83 (m, 2H) 1.91-2.07 (m, 6H) 2.07-2.17 (m, 2H) 2.40-2.50 (m, 2H) 2.76 (br. s., 2H) 2.80-2.95 (m, 3H) 3.12-3.22 (m, 1H) 3.24-3.36 (m, 2H) 3.38 (s, 1H) 3.47-3.66 (m, 2H); MS m/z 334.3 [M+H]⁺ (ESI).

### Intermediate 17

### tert-butyl 4-cyclobutylpiperazine-1-carboxylate

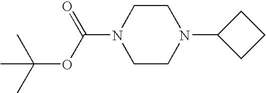

Tert-butyl piperazine-1-carboxylate (13.29 g, 71.34 mmol) and cyclobutanone (5.33 ml, 71.34 mmol) were dissolved in DCE (543 ml) to give a colorless solution. The reaction was stirred at room temperature for 15 min. Sodium triacetoxyborohydride (18.14 g, 85.60 mmol) was added in 2 equal portions, 30 minutes apart. The mixture was stirred overnight at room temperature. The reaction was concentrated under vacuum to half volume and quenched with 1N NaOH (250 mL). EtOAc (500 mL) was added and the phases were separated. The aqueous phase was back extracted with EtOAc (2×250 mL). The combined the organic layers were washed with 1N NaOH (2×300 mL) and brine (1×400 mL), dried over Na₂SO₄, filtered and concentrated under vacuum. The residue was purified by MPLC (330 g silica column, hexane to EtOAc), affording title compound (11.76 g, 95%). MS m/z 241.2 [M+H]⁺ (ESI).

### Intermediate 18

### 1-cyclobutylpiperazine

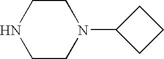

Intermediate 17 (10.75 g, 44.73 mmol) was placed in a 3-neck 1 L round-bottomed flask and dissolved in EtOAc (224 ml) to give a yellow solution. The mixture was cooled to 0° C. and an excess of HCl gas was bubbled into the solution. A white precipitate was formed. After 15 min of HCl gas addition, the mixture was stirred at room temperature for 1 h. The reaction was diluted with ether and stirred overnight. The precipitate was filtered, washed with ether (3×100 mL) and dried overnight, giving 1-cyclobutylpiperazine dihydrochloride (8.79 g, 95%) as a solid. 875 mg (213.2 mmol) of the HCl salt were converted to the title compound by dissolution in 2M NaOH (5 mL) and extraction with EtOAc (150 mL), providing title compound (510 mg, 88%) as a liquid. ¹H NMR (400 MHz, CDCl₃) δ ppm 1.55-1.68 (m, 2H) 1.74-1.90 (m, 2H) 1.91-2.03 (m, 2H) 2.23 (br. s., 4H) 2.64 (quintet, J=7.81 Hz, 1H) 2.83 (t, J=4.88 Hz, 4H); MS m/z 141.1 [M+H]⁺ (ESI).

### Intermediate 19

### tert-butyl 4-cyclobutyl-1,4-diazepane-1-carboxylate

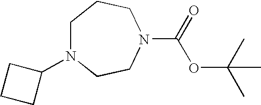

Cyclobutanone (2.011 ml, 26.91 mmol) was added to a solution of tert-butyl 1-homopiperazinecarboxylate (4.76 ml, 24.47 mmol) in DCM (78 mL). The solution was stirred for 1 h at room temperature. Sodium triacetoxyborohydride (6.22 g, 29.36 mmol) was then added by portion over 15 min. The reaction mixture was stirred overnight. The mixture was washed with 2N NaOH (2×30 mL), dried over anhydrous MgSO₄, filtered and concentrated under vacuum. The product was purified by MPLC (silica 120 g, 3% MeOH and 5% acetone in DCM with 0.1N ammonia), providing title compound (4.86 g, 78%). ¹H NMR (400 MHz, CDCl₃) δ ppm 1.46 (s, 9H) 1.54-1.75 (m, 2H) 1.75-1.91 (m, 4H) 1.96-2.11 (m, 2H) 2.33-2.53 (m, 4H) 2.86 (quintet, J=7.91 Hz, 1H) 3.39-3.47 (m, 2H) 3.47-3.54 (m, 2H).

### Intermediate 20

### 1-cyclobutyl-1,4-diazepane

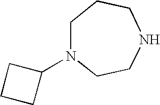

A solution of Intermediate 19 (4.86 g, 19.11 mmol) in DCM (25 mL) was slowly added to TFA (100 mL). The reaction mixture was stirred for 30 min and the solvent was concentrated. The residue was recovered in EtOAc (250 mL), washed with 2N NaOH (3×25 mL), dried over anhydrous MgSO₄ and filtered. The solvent was concentrated under vacuum to provide title compound (2.230 g, 76%) as a gum, which was used in the next step without further purification. ¹H NMR (400 MHz, CD₃OD) δ ppm 1.64-1.79 (m, 2H) 1.88-1.97 (m, 2H) 1.97-2.05 (m, 2H) 2.09-2.19 (m, 2H) 2.71-2.77 (m, 2H) 2.83-2.90 (m, 2H) 3.19 (quintet, J=8.01 Hz, 1H) 3.26-3.30 (m, 4H).

### Intermediate 21

### tert-butyl 4-isopropyl-1,4-diazepane-1-carboxylate

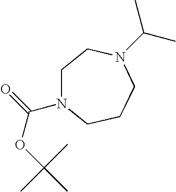

Tert-butyl 1,4-diazepane-1-carboxylate (4.00 mL, 20.55 mmol) was dissolved in MeOH (100 mL). Propan-2-one (7.60 mL, 102.76 mmol), 10% Pd/C (0.875 g, 0.82 mmol) and 4.6 g oven dried 3A sieves were added. The reaction was placed under a H₂ atmosphere and stirred overnight. The mixture was filtered and the filtrate concentrated under vacuum, affording title compound (5.06 g, quantitative), which was used for the next step without further purification. ¹H NMR (400 MHz, CD₃OD) δ ppm 1.02 (d, J=6.64 Hz, 6H) 1.43 (s, 9H) 1.69-1.82 (m, 2H) 2.55-2.64 (m, 2H) 2.64-2.72 (m, 2H) 2.91 (dt, J=13.18, 6.49 Hz, 1H) 3.36-3.47 (m, 4H); MS m/z 243.4 [M+H]⁺ (ESI).

### Intermediate 22

### 1-isopropyl-1,4-diazepane dihydrochloride

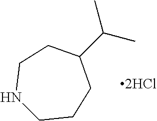

Intermediate 21 (5.06 g, 20.88 mmol) was dissolved in MeOH (50 mL). The solution was cooled to 0° C., and an excess of HCl gas was bubbled through the reaction. The mixture was warmed to room temperature and stirred for 1 h. Volatiles were removed under vacuum and the residue placed under high vacuum overnight, giving title compound (4.298 g, 96%). MS m/z 143.2 [M+H]⁺ (ESI).

### Intermediate 23

### 7-benzyl 2-ethyl 7-azaspiro[3.5]nonane-2,7-dicarboxylate

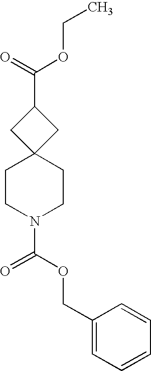

Oxalyl chloride (0.721 mL, 8.24 mmol) was slowly added to a solution of Intermediate 5 (2.50 g, 8.24 mmol) in DCM (60 mL) under a nitrogen atmosphere, at 0° C. The reaction mixture was stirred for 1 h and more oxalyl chloride (0.721 mL, 8.24 mmol) was added. The reaction mixture was stirred for 1 h and oxalyl chloride (0.721 mL, 8.24 mmol) was added again. The reaction mixture was stirred for 15 min and 1 drop of DMF was added. The reaction mixture was stirred for 1 last hour and the solvent was concentrated. The residue was recovered in DCM and quickly added to a solution of ethanol (10 mL) and Et₃N (3.45 mL, 24.72 mmol) in DCM (60 mL) at 0° C. The reaction mixture was allowed to warm to ambient temperature and stirred for 30 min. The solvent was concentrated and the product was recovered in EtOAc. The resulting mixture was washed with H₂O (3×30 mL), dried over anhydrous MgSO₄, filtered and concentrated under reduced pressure. The product was purified on silica gel (120 g) by MPLC using 40% EtOAc in heptane as the eluent to provide title compound (2.55 g, 93%) as an oil. ¹H NMR (400 MHz, CHLOROFORM-d) 8 ppm 1.25 (t, J=7.03 Hz, 3H) 1.57 (d, J=17.97 Hz, 4H) 2.06 (d, J=8.98 Hz, 4H) 3.07 (quin, J=8.79 Hz, 1H) 3.31-3.40 (m, 2H) 3.40-3.48 (m, 2H) 4.13 (q, J=7.03 Hz, 2H) 5.11 (s, 2H) 7.28-7.41 (m, 5H).

### Intermediate 24

### ethyl 7-azaspiro[3.5]nonane-2-carboxylate

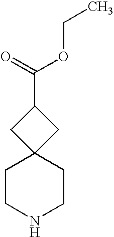

A mixture of Intermediate 23 (2.50 g, 7.54 mmol), Pd—C (0.080 g, 0.75 mmol) and EtOH (70 mL) were shaken in a Parr hydrogenation apparatus, under a 50 psi atmosphere of hydrogen for 4 h. The reaction mixture was filtered over a celite pad and the solvent was concentrated to provide title compound (1.180 g, 79%). The title compound was pure and used in the next step without further purification. ¹H NMR (400 MHz, CHLOROFORM-d) δ ppm 1.25 (t, 3H) 1.57-1.63 (m, 2H) 1.63-1.70 (m, 2H) 2.05 (d, J=8.59 Hz, 4H) 2.72-2.81 (m, 2H) 2.81-2.91 (m, 2H) 3.05 (quin, J=8.79 Hz, 1H) 3.60 (br. s., 1H) 4.13 (q, J=7.03 Hz, 2H); MS m/z 198.0 [M+H]⁺ (ES+).

### Intermediate 25

### ethyl 7-(methylsulfonyl)-7-azaspiro[3.5]nonane-2-carboxylate

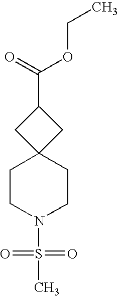

Methanesulfonyl chloride (0.500 mL, 6.41 mmol) was slowly added to a solution of Intermediate 24 (1.15 g, 5.83 mmol) and Et₃N (0.975 mL, 7.00 mmol) in DCM (80 mL) at 0° C. The reaction mixture was allowed to warm to ambient temperature and stirred overnight. The solvent was concentrated and the product was recovered in EtOAc. The resulting mixture was washed with H₂O (3×30 mL), dried over anhydrous MgSO₄, filtered and concentrated under reduced pressure. The product was purified on silica gel (80 g) by MPLC using 40% EtOAc in heptane as the eluent to provide title compound (0.930 g, 57.9%) as a solid. ¹H NMR (400 MHz, CHLOROFORM-d) δ ppm 1.26 (t, J=7.03 Hz, 3H) 1.73 (dt, J=15.23, 5.66 Hz, 4H) 2.07 (d, J=8.59 Hz, 4H) 2.76 (s, 3H) 3.04-3.14 (m, 3H) 3.14-3.21 (m, 2H) 4.14 (q, J=7.03 Hz, 2H); MS m/z 276.2 [M+H]⁺ (ES+).

### Intermediate 26

### 7-(methylsulfonyl)-7-azaspiro[3.5]nonane-2-carboxylic acid

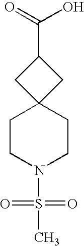

A 2M NaOH solution (2 mL, 4 mmol) was added to a mixture of Intermediate 25 (0.920 g, 3.34 mmol) and H₂O (30 mL). The reaction mixture was stirred for 3 h (substrate dissolution completed) and acidified to pH 1-2 using a 2M hydrochloric acid solution (2.4 mL). The product was extracted using EtOAc (3×30 mL). The combined organic phases were dried over anhydrous Na₂SO₄, filtered and concentrated under reduced pressure to provide title compound (0.780 g, 94%) as a solid. The pure title compound was used in the next step without further purification. ¹H NMR (400 MHz, CHLOROFORM-d) δ ppm 1.69-1.74 (m, 2H) 1.74-1.79 (m, 2H) 2.11 (s, 2H) 2.13 (s, 2H) 2.77 (s, 3H) 3.09-3.14 (m, 2H) 3.14-3.21 (m, 3H); MS m/z 248.2 [M+H]⁺ (ES+).

### Intermediate 27

### (3R)-tert-butyl 3-methyl-4-(7-(methylsulfonyl)-7-azaspiro[3.5]nonane-2-carbonyl)piperazine-1-carboxylate

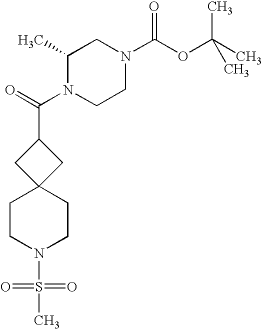

Oxalyl chloride (0.127 mL, 1.46 mmol) was slowly added to a solution of Intermediate 26 (120 mg, 0.49 mmol) in DCM (12 mL) at 0° C. One drop of DMF was added and the reaction mixture was stirred for 4 h. The solvent was concentrated. The residue was quickly recovered in DCM (5 mL) and added to a solution of (R)-tert-butyl 3-methylpiperazine-1-carboxylate (97 mg, 0.49 mmol) and Et₃N (0.338 mL, 2.43 mmol) in DCM (12 mL) at 0° C. The reaction mixture was allowed to warm to ambient temperature and stirred for 1 h. The solvent was concentrated and the product was purified on silica gel (24 g) by MPLC using 5% MeOH and 10% acetone in DCM as the eluent to provide title compound (111 mg, 53.3%) as a solid. ¹H NMR (400 MHz, CHLOROFORM-d) δ ppm 1.14 (d, J=7.03 Hz, 2H) 1.22 (d, J=6.64 Hz, 2H) 1.47 (s, 9H) 1.63-1.72 (m, 2H) 1.72-1.84 (m, 2H) 1.92-2.30 (m, 4H) 2.76 (s, 3H) 2.78-3.03 (m, 2H) 3.11 (t, J=5.66 Hz, 2H) 3.14-3.29 (m, 4H) 3.30-3.42 (m, 1H) 3.84 (br. s., 1H) 4.30-4.82 (m, 1H); MS m/z 430.3 [M+H]⁺ (ES+).

### Intermediate 28

### ((R)-2-methylpiperazin-1-yl)(7-(methylsulfonyl)-7-azaspiro[3.5]nonan-2-yl)methanone 2,2,2-trifluoroacetate

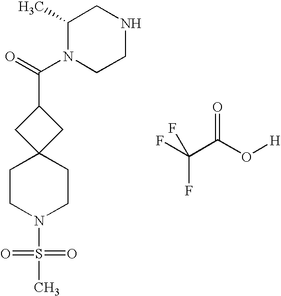

TFA (12 mL) was added to Intermediate 27 (110 mg, 0.26 mmol) and the mixture was stirred for 4 h. The solvent was concentrated to provide title compound (110 mg, 97%) as a gum. The product was used in the next step without further purification. MS m/z 330.0 [M+H]⁺ (ES+).

### Intermediate 29

### (3S)-tert-butyl 3-methyl-4-(7-(methylsulfonyl)-7-azaspiro[3.5]nonane-2-carbonyl)piperazine-1-carboxylate

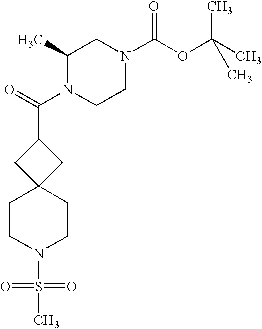

Oxalyl chloride (0.127 mL, 1.46 mmol) was slowly added to a solution of Intermediate 26 (120 mg, 0.49 mmol) in DCM (12 mL) at 0° C. One drop of DMF was added and the reaction mixture was stirred for 4 h. The solvent was concentrated. The residue was quickly recovered in DCM (5 mL) and added to a solution of (S)-tert-butyl 3-methylpiperazine-1-carboxylate (97 mg, 0.49 mmol) and Et₃N (0.338 mL, 2.43 mmol) in DCM (12 mL) at 0° C. The reaction mixture was allowed to warm to ambient temperature and stirred for 1 hr. The solvent was concentrated and the product was purified on silica gel (24 g) by MPLC using 5% MeOH and 10% acetone in DCM as the eluent to provide title compound (118 mg, 56.6%) as a solid; ¹H NMR (400 MHz, CHLOROFORM-d) δ ppm 1.14 (d, J=7.03 Hz, 2H) 1.22 (d, J=6.64 Hz, 2H) 1.47 (s, 9H) 1.61-1.73 (m, 2H) 1.73-1.83 (m, 2H) 1.95-2.26 (m, 4H) 2.76 (s, 4H) 2.83-3.03 (m, 1H) 3.05-3.14 (m, 3H) 3.14-3.23 (m, 3H) 3.23-3.42 (m, 1H) 3.84 (br. s., 1H) 4.31-4.80 (m, 1H); MS m/z 430.2 [M+H]⁺ (ES+).

### Intermediate 30

### ((S)-2-methylpiperazin-1-yl)(7-(methylsulfonyl)-7-azaspiro[3.5]nonan-2-yl)methanone 2,2,2-trifluoroacetate

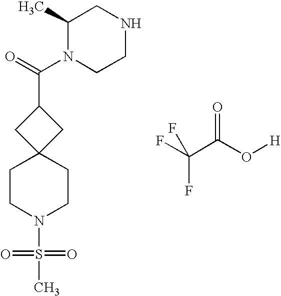

TFA (12 mL) was added to Intermediate 29 (110 mg, 0.26 mmol) and the mixture was stirred for 4 h. The solvent was concentrated to provide title compound (114 mg, 100%) as a gum. The product was used in the next step without further purification. MS m/z 330.0 [M+H]⁺ (ES+).

### Intermediate 31

### (S)-tert-butyl 3-methylpiperazine-1-carboxylate

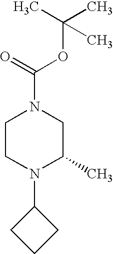

Tert-butyl (3S)-3-methylpiperazine-1-carboxylate (5.0 g, 24.96 mmol) was dissolved in DCM (50 mL, 10 parts) under nitrogen. Cyclobutanone (1.98 g, 27.46 mmol) was added to the substrate solution and the resultant mixture was stirred for 45 min at ambient temperature. The resultant mixture was cooled to 0-5° C. in an ice-water bath. Sodium triacetoxyborohydride (6.35 g, 29.95 mmol) was added to the cooled solution and stirred at 0-5° C. for another 30 min. The reaction mixture was allowed to warm to room temperature and was stirred over 2 days under nitrogen. The reaction mixture was cooled to 0-5° C. in an ice-water bath. Aqueous NaOH (1 M, 100 mL) was gradually added to the reaction mixture and stirring was continued for 5 min. The organic layer was collected and the aqueous layer was extracted with DCM (3×75 mL). The combined organic phases were washed with brine (100 mL), dried over anhydrous magnesium sulphate and filtered. Evaporation of the solvent under reduced pressure provided title product (6.35 g, 100%) as an oil. ¹H NMR (400 MHz, CDCl₃) δ 0.97 (d, J=6.3 Hz, 3H) 1.45 (s, 9H) 1.57-1.73 (m, 2H) 1.88 (quintet, J=9.4 Hz, 1H) 1.96 (m, 2H) 2.07 (m, 2H) 2.38-2.53 (m, 1H) 2.60 (m, 1H) 3.00 (m, 1H) 3.00-3.20 (m, 1H), 3.20-3.63 (m, 3H).

### Intermediate 32

### (S)-1-cyclobutyl-2-methylpiperazine dihydrochloride

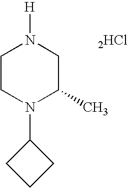

Intermediate 31 (16.77 g, 65.93 mmol) was dissolved in dioxane (72.5 mL, 6.5 parts) under nitrogen. Hydrochloric acid in dioxane (4 M, 82.5 mL, 329.65 mmol) was added to the reaction. The resulting mixture was stirred at ambient temperature for 30 min, then heated to 60° C. and stirred for 3 h. The reaction mixture was cooled to ambient temperature and the solvent was evaporated under reduced pressure on a rotary evaporator. The crude product obtained was triturated with DCM (150 mL) to form a suspension. The solid was collected by filtration and dried under vacuum at 55° C. to afford title compound (14.80 g, 98%). ¹H NMR (400 MHz, DMSO-d₆) δ 1.35 (d, J=6.6 Hz, 3H) 1.69 (m, 2H) 2.00-2.36 (m, 3H) 2.70 (m, 1H) 2.98-3.71 (m, 7H) 3.89 (m, 1H) 9.1-10.7 (m, 2H, exchanged with D₂O) 11.92-12.71 (m, 1H, exchanged with D₂O).

### Intermediate 33

### methyl 3-bromoisonicotinate

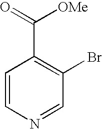

H₂SO₄ (0.5 mL) was added to a solution of 3-bromoisonicotinic acid (500 mg, 2.48 mmol) in MeOH (10 mL). The resulting solution was heated at reflux overnight. The mixture was cooled to 0° C. and a solution of 5% NaHCO₃ (5 mL) was added. The aqueous layer was basified to pH=7-8 with 50% aqueous NaOH. It was then extracted with DCM (3×). The combined organic extracts were washed with brine, dried over MgSO₄, filtered and concentrated under reduced pressure to afford title crude product (465 mg, 87%) as an oil. ¹H NMR (300 MHz, CDCl₃) δ ppm 3.96 (s, 3H) 7.61 (d, J=5.10 Hz, 1H) 8.61 (d, J=5.10 Hz, 1H) 8.85 (s, 1H).

### Intermediate 34

### methyl 3-cyanoisonicotinate

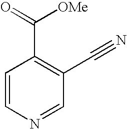

In a flame dried microwave tube, Zn(CN)₂ (54 mg, 0.463 mmol) and Pd(PPh₃)₄ (16 mg, 0.014 mmol) were added to a solution of Intermediate 33 (100 mg, 0.463 mmol) in DMAc (4 mL). Nitrogen was bubbled through the solution for 5 min and then the tube was sealed. The reaction mixture was heated in a microwave apparatus to 180° C. for 3 min. The resulting mixture was purified on a reverse phase silica gel cartridge (25 g) with H₂O and acetonitrile from 90:10 to 80:20. The product obtained was then purified by flash chromatography on silica gel, eluting with mixtures of hexanes and EtOAc (92:8 to 30:70) to afford title product (74 mg, 99%) as a solid. ¹H NMR (300 MHz, CDCl₃) δ ppm 4.06 (s, 3H) 7.98 (d, J=5.10 Hz, 1H) 8.96 (d, J=5.10 Hz, 1H) 9.06 (s, 1H).

### Intermediate 35

### 3-cyanoisonicotinic acid

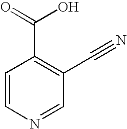

A solution of 2 N NaOH (0.62 mL) was added to a solution of Intermediate 34 (155 mg, 0.96 mmol) in MeOH (2.5 mL). The resulting solution was stirred at room temperature for 1 h. A solution of 2 N HCl (0.62 mL) was added and the resulting mixture was concentrated with silica gel under reduced pressure. The crude product was then purified by flash chromatography on silica gel, eluting with mixtures of DCM and MeOH (75:25) followed by HPLC with water to afford title compound (114 mg, 80%) as a solid. ¹H NMR (300 MHz, CDCl₃) δ ppm 7.99 (d, J=5.20 Hz, 1H) 8.90 (d, J=5.10 Hz, 1H) 9.02 (s, 1H).

What is claimed is:

1. A compound of formula I, or pharmaceutically acceptable salts of formula I, or mixtures thereof: 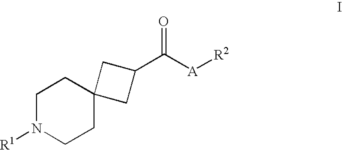
wherein
A is 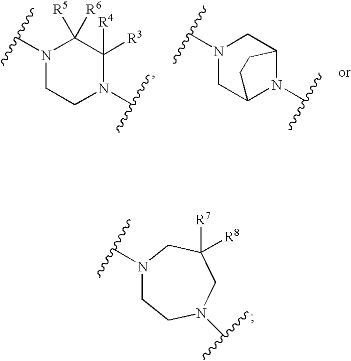
R¹ is aryl, 5- or 6-membered heteroaryl, —S(═O)₂R⁹, —C(═O)R¹⁰, or —C(═O)NR¹¹R¹²;
R² is C₃₋₆cycloalkyl or C₁₋₆alkyl;
R³, R⁴, R⁵, R⁶, R⁷, and R⁸ are each, independently, selected from hydrogen and —C₁-C₃alkyl;
R⁹, R¹⁰ and R¹¹ are each, independently, selected from C₁₋₆alkyl, 4-membered heterocycloalkyl, 5-membered heterocycloalkyl, 6-membered heterocycloalkyl, 7-membered heterocycloalkyl, C₃₋₇cycloalkyl, aryl, 5-membered heteroaryl, 6-membered heteroaryl, —(C₁₋₃alkyl)-(5-membered heteroaryl), and —(C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;
R¹² is H or C₁₋₆alkyl; and
R¹³ and R¹⁴ are each, independently, selected from H and C₁₋₃alkyl.

2. A compound according to claim 1, or pharmaceutically acceptable salts thereof or mixtures thereof, wherein A is

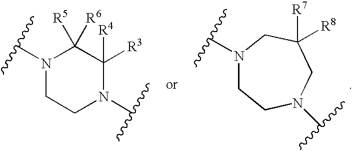

3. A compound according to claim 1, or pharmaceutically acceptable salts thereof or mixtures thereof, wherein A is

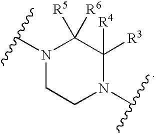

4. A compound according to claim 1, or pharmaceutically acceptable salts thereof or mixtures thereof, wherein A is

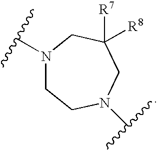

5. A compound according to claims 1-4, or pharmaceutically acceptable salts thereof or mixtures thereof, wherein R¹ is phenyl, 6-membered heteroaryl, —S(═O)₂R⁹, —C(═O)R¹⁰, or —C(═O)NR¹¹R¹².

6. A compound according to claims 1-5, or pharmaceutically acceptable salts thereof or mixtures thereof, wherein R¹ is phenyl, pyridinyl, pyrazinyl, pyrimidinyl, —S(═O)₂R⁹, —C(═O)R¹⁰, or —C(═O)NR¹¹R¹².

7. A compound according to claims 1-6, or pharmaceutically acceptable salts thereof or mixtures thereof, wherein R⁹ is C₁₋₃alkyl or phenyl.

8. A compound according to claims 1-6, or pharmaceutically acceptable salts thereof or mixtures thereof, wherein R⁹ is an aryl substituted by 1, 2, or 3 substituents selected from halo.

9. A compound according to claims 1-8, or pharmaceutically acceptable salts thereof or mixtures thereof, wherein R¹⁰ is C₃₋₆cycloalkyl, aryl, 6-membered heteroaryl, or —(C₁₋₃alkyl)-(6-membered heteroaryl).

10. A compound according to claims 1-9, or pharmaceutically acceptable salts thereof or mixtures thereof, wherein R¹⁰ is cyclohexyl, pyridinyl, phenyl, nicotinonitrile, methylpyridine, or ethylpyridine.

11. A compound according to claims 1-9, or pharmaceutically acceptable salts thereof or mixtures thereof, wherein R¹⁰ is an aryl or 6-membered heteroaryl, wherein said aryl or heteroaryl are each, independently, substituted by 1 or 2 substituents selected from halo, cyano, C₁₋₃alkyl, and C₁₋₃alkoxy.

12. A compound according to claims 1-9 or 11, or pharmaceutically acceptable salts thereof or mixtures thereof, wherein R¹⁰ is an aryl substituted by 1 or 2 substituents selected from cyano.

13. A compound according to claims 1-9 or 11, or pharmaceutically acceptable salts thereof or mixtures thereof, wherein R¹⁰ is a 6-membered heteroaryl substituted by 1 or 2 substituents selected from halo, cyano, C₁₋₃alkyl, and C₁₋₃alkoxy.

14. A compound according to claims 1-9, 11, or 13, or pharmaceutically acceptable salts thereof or mixtures thereof, wherein R¹⁰ is a 6-membered heteroaryl substituted by 1 or 2 substituents selected from methyl, methoxy, ethyl, cyano, fluoro, and chloro.

15. A compound according to claims 1-14, or pharmaceutically acceptable salts thereof or mixtures thereof, wherein R¹¹ and R¹² are each, independently, selected from H, phenyl, and C₁₋₃alkyl.

16. A compound according to claims 1-15, or pharmaceutically acceptable salts thereof or mixtures thereof, wherein R³, R⁴, R⁵, R⁶, R⁷, and R⁸ are each, independently, hydrogen.

17. A compound according to claims 1-16, or pharmaceutically acceptable salts thereof or mixtures thereof, wherein R³, R⁴, R⁵, R⁶, R⁷, and R⁸ are each, independently, hydrogen or CH₃.

18. At least one compound selected from:
(4-cyclobutylpiperazin-1-yl)(7-(pyridin-3-yl)-7-azaspiro[3.5]nonan-2-yl)methanone;
(4-cyclobutylpiperazin-1-yl)(7-(pyridin-2-yl)-7-azaspiro[3.5]nonan-2-yl)methanone;
(4-cyclobutylpiperazin-1-yl)(7-phenyl-7-azaspiro[3.5]nonan-2-yl)methanone;
(4-cyclobutylpiperazin-1-yl)(7-(isopropylsulfonyl)-7-azaspiro[3.5]nonan-2-yl)methanone;
(4-cyclobutylpiperazin-1-yl)(7-(methylsulfonyl)-7-azaspiro[3.5]nonan-2-yl)methanone;
(4-cyclobutylpiperazin-1-yl)(7-(phenylsulfonyl)-7-azaspiro[3.5]nonan-2-yl)methanone;
(4-cyclobutylpiperazin-1-yl)(7-(cyclohexanecarbonyl)-7-azaspiro[3.5]nonan-2-yl)methanone; (4-cyclobutylpiperazin-1-yl)(7-(3-ethylisonicotinoyl)-7-azaspiro[3.5]nonan-2-yl)methanone; (7-(5-chloronicotinoyl)-7-azaspiro[3.5]nonan-2-yl)(4-cyclobutylpiperazin-1-yl)methanone; (7-(4-chloronicotinoyl)-7-azaspiro[3.5]nonan-2-yl)(4-cyclobutylpiperazin-1-yl)methanone; (4-cyclobutylpiperazin-1-yl)(7-(2,4-dimethylnicotinoyl)-7-azaspiro[3.5]nonan-2-yl)methanone;
(4-cyclobutylpiperazin-1-yl)(7-(6-methylnicotinoyl)-7-azaspiro[3.5]nonan-2-yl)methanone; (4-cyclobutylpiperazin-1-yl)(7-(2-methylnicotinoyl)-7-azaspiro[3.5]nonan-2-yl)methanone; (4-cyclobutylpiperazin-1-yl)(7-(4-methylnicotinoyl)-7-azaspiro[3.5]nonan-2-yl)methanone; 6(2-(4-cyclobutylpiperazine-1-carbonyl)-7-azaspiro[3.5]nonane-7-carbonyl)nicotinonitrile;
(4-cyclobutylpiperazin-1-yl)(7-(3,5-difluoropicolinoyl)-7-azaspiro[3.5]nonan-2-yl)methanone;
(7-(4-chloropicolinoyl)-7-azaspiro[3.5]nonan-2-yl)(4-cyclobutylpiperazin-1-yl)methanone; (4-cyclobutylpiperazin-1-yl)(7-(6-methylpicolinoyl)-7-azaspiro[3.5]nonan-2-yl)methanone; (4-cyclobutylpiperazin-1-yl)(7-(3-methylpicolinoyl)-7-azaspiro[3.5]nonan-2-yl)methanone; (4-cyclobutylpiperazin-1-yl)(7-picolinoyl-7-azaspiro[3.5]nonan-2-yl)methanone;
(4-cyclobutylpiperazin-1-yl)(7-nicotinoyl-7-azaspiro[3.5]nonan-2-yl)methanone;
(4-cyclobutylpiperazin-1-yl)(7-isonicotinoyl-7-azaspiro[3.5]nonan-2-yl)methanone;
(4-cyclobutylpiperazin-1-yl)(7-(3-methylisonicotinoyl)-7-azaspiro[3.5]nonan-2-yl)methanone;
(4-cyclobutylpiperazin-1-yl)(7-(2-methylisonicotinoyl)-7-azaspiro[3.5]nonan-2-yl)methanone;
(4-cyclobutylpiperazin-1-yl)(7-(5-methylnicotinoyl)-7-azaspiro[3.5]nonan-2-yl)methanone; (4-cyclobutylpiperazin-1-yl)(7-(2-methoxyisonicotinoyl)-7-azaspiro[3.5]nonan-2-yl)methanone;
1-(2-(4-cyclobutylpiperazine-1-carbonyl)-7-azaspiro[3.5]nonan-7-yl)-2-(pyridin-3-yl)ethanone;
1-(2-(4-cyclobutylpiperazine-1-carbonyl)-7-azaspiro[3.5]nonan-7-yl)-2-(pyridin-4-yl)ethanone;
1-(2-(4-cyclobutylpiperazine-1-carbonyl)-7-azaspiro[3.5]nonan-7-yl)-3-(pyridin-4-yl)propan-1-one;
(7-benzoyl-7-azaspiro[3.5]nonan-2-yl)(4-cyclobutylpiperazin-1-yl)methanone;
3-(2-(4-cyclobutylpiperazine-1-carbonyl)-7-azaspiro[3.5]nonane-7-carbonyl)benzonitrile;
2-(4-cyclobutylpiperazine-1-carbonyl)-N-phenyl-7-azaspiro[3.5]nonane-7-carboxamide;
(4-isopropylpiperazin-1-yl)(7-(methylsulfonyl)-7-azaspiro[3.5]nonan-2-yl)methanone;
(4-isopropylpiperazin-1-yl)(7-(2-methylisonicotinoyl)-7-azaspiro[3.5]nonan-2-yl)methanone; (7-isonicotinoyl-7-azaspiro[3.5]nonan-2-yl)(4-isopropylpiperazin-1-yl)methanone;
(4-isopropylpiperazin-1-yl)(7-nicotinoyl-7-azaspiro[3.5]nonan-2-yl)methanone;
2-(4-isopropylpiperazine-1-carbonyl)-N,N-dimethyl-7-azaspiro[3.5]nonane-7-carboxamide; (4-cyclobutyl-1,4-diazepan-1-yl)(7-(4-fluorophenylsulfonyl)-7-azaspiro[3.5]nonan-2-yl)methanone;
(4-cyclobutyl-1,4-diazepan-1-yl)(7-(3-fluorophenylsulfonyl)-7-azaspiro[3.5]nonan-2-yl)methanone;
(4-cyclobutyl-1,4-diazepan-1-yl)(7-(2-fluorophenylsulfonyl)-7-azaspiro[3.5]nonan-2-yl)methanone;
(4-cyclobutyl-1,4-diazepan-1-yl)(7-(phenylsulfonyl)-7-azaspiro[3.5]nonan-2-yl)methanone; (4-cyclobutyl-1,4-diazepan-1-yl)(7-picolinoyl-7-azaspiro[3.5]nonan-2-yl)methanone;
(4-cyclobutyl-1,4-diazepan-1-yl)(7-(2-methylisonicotinoyl)-7-azaspiro[3.5]nonan-2-yl)methanone;
(4-cyclobutyl-1,4-diazepan-1-yl)(7-(3-methylisonicotinoyl)-7-azaspiro[3.5]nonan-2-yl)methanone;
(4-cyclobutyl-1,4-diazepan-1-yl)(7-isonicotinoyl-7-azaspiro[3.5]nonan-2-yl)methanone;
(4-cyclobutyl-1,4-diazepan-1-yl)(7-(5-methylnicotinoyl)-7-azaspiro[3.5]nonan-2-yl)methanone;
(4-cyclobutyl-1,4-diazepan-1-yl)(7-nicotinoyl-7-azaspiro[3.5]nonan-2-yl)methanone;
(7-benzoyl-7-azaspiro[3.5]nonan-2-yl)(4-cyclobutyl-1,4-diazepan-1-yl)methanone;
4-(2-(4-cyclobutyl-1,4-diazepane-1-carbonyl)-7-azaspiro[3.5]nonane-7-carbonyl)benzonitrile;
3-(2-(4-cyclobutyl-1,4-diazepane-1-carbonyl)-7-azaspiro[3.5]nonane-7-carbonyl)benzonitrile;
2-(2-(4-cyclobutyl-1,4-diazepane-1-carbonyl)-7-azaspiro[3.5]nonane-7-carbonyl)benzonitrile;
(4-isopropyl-1,4-diazepan-1-yl)(7-(methylsulfonyl)-7-azaspiro[3.5]nonan-2-yl)methanone; (4-isopropyl-1,4-diazepan-1-yl)(7-(2-methylisonicotinoyl)-7-azaspiro[3.5]nonan-2-yl)methanone;
(7-isonicotinoyl-7-azaspiro[3.5]nonan-2-yl)(4-isopropyl-1,4-diazepan-1-yl)methanone;
(4-isopropyl-1,4-diazepan-1-yl)(7-nicotinoyl-7-azaspiro[3.5]nonan-2-yl)methanone;
2-(4-isopropyl-1,4-diazepane-1-carbonyl)-N,N-dimethyl-7-azaspiro[3.5]nonane-7-carboxamide;
(4-cyclobutyl-6,6-dimethyl-1,4-diazepan-1-yl)(7-(2-methylisonicotinoyl)-7-azaspiro[3.5]nonan-2-yl)methanone;
(4-cyclopentylpiperazin-1-yl)(7-(methylsulfonyl)-7-azaspiro[3.5]nonan-2-yl)methanone; ((S)-4-isopropyl-3-methylpiperazin-1-yl)(7-(methylsulfonyl)-7-azaspiro[3.5]nonan-2-yl)methanone;
((R)-4-cyclobutyl-2-methylpiperazin-1-yl)(7-(methylsulfonyl)-7-azaspiro[3.5]nonan-2-yl)methanone;
4-(2-(4-cyclobutylpiperazine-1-carbonyl)-7-azaspiro[3.5]nonane-7-carbonyl)nicotinonitrile; (4-cyclobutylpiperazin-1-yl)(7-(pyrazin-2-yl)-7-azaspiro[3.5]nonan-2-yl)methanone;
(4-cyclobutylpiperazin-1-yl)(7-(pyridin-4-yl)-7-azaspiro[3.5]nonan-2-yl)methanone; and
(4-cyclobutylpiperazin-1-yl)(7-(pyrimidin-5-yl)-7-azaspiro[3.5]nonan-2-yl)methanone;
and pharmaceutically acceptable salts thereof and mixtures thereof.

19. At least one compound according to any one of claims 1-18 for use as a medicament.

20. The use of a compound according to any one of claims 1-18 in the manufacture of a medicament for the therapy of at least one disorder selected from cognitive deficit in schizophrenia, narcolepsy, obesity, attention deficit hyperactivity disorder, and Alzheimer's disease.

21. The use according to claim 20, wherein said disorder is cognitive deficit in schizophrenia.

22. The use according to claim 20, wherein said disorder is narcolepsy.

23. The use according to claim 20, wherein said disorder is obesity.

24. The use according to claim 20, wherein said disorder is attention deficit hyperactivity disorder.

25. The use according to claim 20, wherein said disorder is Alzheimer's disease.

26. A pharmaceutical composition comprising a therapeutically effective amount of at least one compound according to any one of claims 1-18, or pharmaceutically acceptable salts thereof or mixtures thereof, and a pharmaceutically acceptable carrier and/or diluent.

27. A method for treating at least one disorder selected from cognitive deficit in schizophrenia, narcolepsy, obesity, attention deficit hyperactivity disorder, and Alzheimer's disease in a warm-blooded animal, comprising administering to said animal in need of such treatment a pharmaceutical composition according to claim 26.

28. A method for treating at least one disorder selected from cognitive deficit in schizophrenia, narcolepsy, obesity, attention deficit hyperactivity disorder, and Alzheimer's disease in a warm-blooded animal, comprising administering to said animal in need of such treatment a therapeutically effective amount of at least one compound according to any one of claims 1-18.

29. The method according to claim 28, wherein said disorder is cognitive deficit in schizophrenia.

30. The method according to claim 28, wherein said disorder is Alzheimer's disease.

31. The method according to claim 28, wherein said disorder is obesity.

32. The method according to claim 28, wherein said disorder is narcolepsy.

33. The method according to claim 28, wherein said disorder is attention deficit hyperactivity disorder.

34. A method for treating a disorder in which modulating the histamine H3 receptor is beneficial comprising administering to a warm-blooded animal in need of such treatment a therapeutically effective amount of at least one compound according to formula I, or pharmaceutically acceptable salts of formula I, or mixtures thereof: 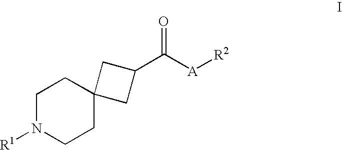
wherein
A is 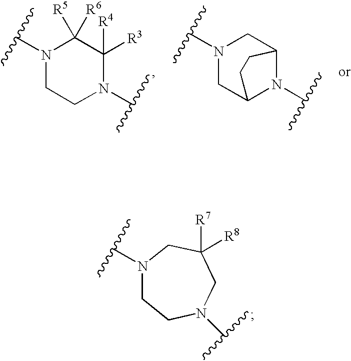
R¹ is aryl, 5- or 6-membered heteroaryl, —S(═O)₂R⁹, —C(═O)R¹⁰, or —C(═O)NR¹¹R¹²;
R² is C₃₋₆cycloalkyl or C₁₋₆alkyl;
R³, R⁴, R⁵, R⁶, R², and R⁸ are each, independently, selected from hydrogen and —C₁-C₃alkyl;
R⁹, R¹⁰ and R¹¹ are each, independently, selected from C₁₋₆alkyl, 4-membered heterocycloalkyl, 5-membered heterocycloalkyl, 6-membered heterocycloalkyl, 7-membered heterocycloalkyl, C₃₋₇cycloalkyl, aryl, 5-membered heteroaryl, 6-membered heteroaryl, —(C₁₋₃alkyl)-(5-membered heteroaryl), and (C₁₋₃alkyl)-(6-membered heteroaryl), wherein said aryl and heteroaryl are each, independently, optionally substituted by 1, 2, or 3 substituents selected from halo, —CF₃, cyano, C₁₋₃alkyl, C₁₋₃alkoxy, and —C(═O)NR¹³R¹⁴;
R¹² is H or C₁₋₆alkyl; and
R¹³ and R¹⁴ are each, independently, selected from H and C₁₋₃alkyl.

35. The method according to claim 34, wherein said at least one compound of formula I is an inverse agonist of at least one histamine H3 receptor.

36. The method according to claim 34, wherein said at least one compound of formula I is an antagonist of at least one histamine H3 receptor.

37. The method according to claim 34, wherein said disorder is selected from cognitive deficit in schizophrenia, narcolepsy, obesity, attention deficit hyperactivity disorder, and Alzheimer's disease.
